(19) United States
(12) Patent Application Publication
(10) Pub. No.: US 2022/0146508 A1
(43) Pub. Date: May 12, 2022

(54) RAPID DIAGNOSTIC TEST

(71) Applicant: Detect, Inc., Guilford, CT (US)

(72) Inventors: Todd Roswech, Ivoryton, CT (US); Jonathan M. Rothberg, Miami Beach, FL (US); Isaac Bean, Colorado Springs, CO (US); Monica Florence Caravias, San Francisco, CA (US); Matthew Dyer, Spring, TX (US)

(73) Assignee: Detect, Inc., Guilford, CT (US)

(21) Appl. No.: 17/518,693

(22) Filed: Nov. 4, 2021

Related U.S. Application Data
(60) Provisional application No. 63/110,783, filed on Nov. 6, 2020.

(51) Int. Cl. G01N 33/543; B01L 3/00

(52) U.S. Cl. CPC G01N 33/54388; B01L 3/5023; B01L 2300/0832; B01L 2400/06; B01L 2300/0672; B01L 2200/0689

(57) ABSTRACT

Provided herein, in some embodiments, are rapid diagnostic tests to detect one or more target nucleic acid sequences (e.g., a nucleic acid sequence of one or more pathogens). In some embodiments, the pathogens are viral, bacterial, fungal, parasitic, or protozoan pathogens, such as SARS-CoV-2 or an influenza virus. Further embodiments provide methods of detecting genetic abnormalities. Diagnostic tests comprising a sample-collecting component, one or more reagents (e.g., lysis reagents, nucleic acid amplification reagents), and a detection component (e.g., a component comprising a lateral flow assay strip and/or a colorimetric assay) are provided.

### RELATED APPLICATIONS

This application claims the benefit of priority under 35 U.S.C. § 119(e) of U.S. Provisional Application Ser. No. 63/110,783, filed Nov. 6, 2020, the disclosure of which is incorporated herein by reference in its entirety.

### FIELD

The present invention generally relates to diagnostic devices, systems, and methods for detecting the presence of a target nucleic acid sequence.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Dec. 31, 2021, is named H0966.70041US01-SEQ-TRD and is 4,695 bytes in size.

### BACKGROUND

The ability to rapidly diagnose diseases—particularly highly infectious diseases—is critical to preserving human health. As one example, the high level of contagiousness, the high mortality rate, and the lack of a treatment or vaccine for the coronavirus disease 2019 (COVID-19) have resulted in a pandemic that has already infected millions and killed hundreds of thousands of people. The existence of rapid, accurate COVID-19 diagnostic tests could allow infected individuals to be quickly identified and isolated, which could assist with containment of the disease. In the absence of such diagnostic tests, COVID-19 may continue to spread unchecked throughout communities.

### SUMMARY

Provided herein are a number of diagnostic tests useful for detecting target nucleic acid sequences. The tests, as described herein, are able to be performed in a point-of-care (POC) setting or home setting without specialized equipment. Therefore, in some aspects, the disclosure provides a diagnostic test including a reservoir containing a solution as well as a later flow assay strip. The diagnostic test includes at least one seal positioned in a fluidic channel between the reservoir and the lateral flow assay strip, where the seal prevents fluid flow from the reservoir to the lateral flow assay strip until the seal is opened.

In some embodiments, a detection component of a diagnostic test includes a first reservoir for containing a first solution, a lateral flow assay strip, a receptacle configured to receive and fluidly connect to a second reservoir containing a sample, where fluidly connecting the second reservoir to the receptacle allows the sample to flow from the second reservoir to the lateral flow assay strip, and a seal positioned between the first reservoir and the lateral flow assay strip, where fluidly connecting the second reservoir to the receptacle opens the seal to allow the first solution to flow from the first reservoir to the lateral flow assay strip.

In some embodiments, a detection component of a diagnostic test, includes a vial including an internal volume, the vial including a first reservoir for containing a sample, a second reservoir for containing a first solution, and a seal positioned between the first reservoir and the second reservoir, where the seal is configured to be opened to fluidly connect the first reservoir and the second reservoir. The detection component includes a third reservoir configured to receive the sample and the first solution when the seal is opened, a lateral flow assay strip fluidly connected to the third reservoir, and an actuator configured to selectively open the seal.

In some embodiments, a method of performing a diagnostic test includes depositing a sample in a first reservoir, moving the first reservoir into a receptacle of a detection component, opening a seal positioned between a second reservoir containing a first solution and a lateral flow assay strip to allow the first solution to flow toward the lateral flow assay strip in response to moving the first reservoir into the receptacle, and fluidly connecting the first reservoir with the receptacle to allow the sample to flow toward the lateral flow assay strip.

In some embodiments, a method of performing a diagnostic test includes depositing a sample in a first reservoir of a vial, depositing a first solution in a second reservoir of the vial, wherein the first reservoir and the second reservoir are separated by a seal that is openable to fluidly connect the first reservoir and the second reservoir, placing the vial in a receptacle of a detection component, opening the seal with an actuator to allow the sample and the first solution to flow to a third reservoir of the vial, mixing the first solution and the sample in the third reservoir, and allowing the mixed first solution and the sample to flow to a lateral flow assay strip.

In some embodiments, a method of performing a diagnostic test includes depositing a sample in a first reservoir of a vial, depositing a first solution in a second reservoir of the vial, wherein the first reservoir and the second reservoir are separated by a first seal that is openable to fluidly connect the first reservoir and the second reservoir, placing the vial in a receptacle of a detection component, opening the first seal with an actuator to allow the sample to flow to the second reservoir to mix with the first solution, and opening a second seal positioned between the second reservoir and a third reservoir of the vial, wherein the third reservoir is in fluid communication with a lateral flow assay strip.

In some embodiments, a method of making a diagnostic test includes filling a first reservoir with a first solution, where the first reservoir is disposed in a housing, placing a lateral flow assay strip in the housing, placing a seal positioned between the first reservoir and the lateral flow assay strip, wherein the seal is configured to allow the first solution to flow from the first reservoir to the lateral flow assay strip when opened, and providing a vial for taking a sample from a patient, where the vial is configured to fluidly connect to the housing, and wherein the fluidly connecting the vial to the housing opens the seal.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1F show, according to some embodiments, a process of performing a diagnostic test for the presence of one or more nucleic acid sequences;

FIGS. 2A-2C show, according to some embodiments, a detection component comprising a “chimney” and a blister;

FIG. 3 shows, according to some embodiments, a flow chart for a method of performing a diagnostic test;

FIGS. 4A-4B show, according to some embodiments, a detection component comprising an actuator;

FIG. 5 shows, according to some embodiments, a flow chart for a method of performing a diagnostic test;

FIGS. 6A-6C show, according to some embodiments, a detection component comprising a plurality of actuators;

FIG. 7 shows, according to some embodiments, a flow chart for a method of performing a diagnostic test;

FIG. 8 shows, according to some embodiments, a flow chart for a method of making a diagnostic test;

FIG. 9A shows, according to some embodiments, an exploded view of a detection component comprising a “chimney”;

FIG. 9B shows, according to some embodiments, a cross-section of the detection component of FIG. 9A taken along line 9A-9A; and

FIGS. 10A-10B show diagnostic kits comprising a sample-collecting component, a reaction tube, a detection component, and a heater, according to some embodiments.

### DETAILED DESCRIPTION

Conventional nucleic acid tests for various diseases requires trained medical professional to collect samples and process those samples in a sterile environment in a laboratory. Such a process is time consuming, resulting in a delay in providing results to patients. Additionally, such tests require a patient to visit a location where a sample may be collected and transported in a sterile manner to an appropriate processing location. Travel to and from locations may risk spread of the disease being tested for and may inadvertently expose medical personnel to the disease.

In view of the above, the inventors have recognized the benefits of a detection component of a rapid diagnostic test that is usable by a user who may not be a trained medical professional. In particular, the inventors have recognized the benefits of a detection component configured to receive a fluid sample employing fluid reservoirs having seals that may be easily punctured to fluidly connect various elements of the rapid diagnostic test in sequence while maintaining sterility. For example, the inventors have recognized the benefits of a detection component including a blister containing or fluidly sealing a diluent, which may be fluidly connected to a sample when the blister is ruptured. Such a detection component may allow users to perform tests at home and receive results in a rapid manner without necessarily requiring input from trained medical staff. Telemedicine or applications on a personal device may be employed to further enhance the usability of a rapid diagnostic test and the detection component, such that a variety of diseases such as COVID-19, influenza, (or any target nucleic acid) may be tested for in a home environment. Of course, a diagnostic test according to exemplary embodiments described herein may be administered by trained medical staff in an at-home setting or in a point-of-care setting, as the present disclosure is not so limited. A diagnostic test according to exemplary embodiments described herein may be easier to implement and use for medical staff in a point-of-care setting than conventional testing methods. Furthermore, in some embodiments, different portions of a diagnostic test process may occur in different locations. For example, taking a sample may occur in an at-home environment, while a detection process may occur in a point-of-care setting.

Self-Administrable Test with Detection Component

The present disclosure provides diagnostic devices, systems, and methods for rapidly (and in an at-home environment) detecting one or more target nucleic acid sequences (e.g., a nucleic acid sequence of a pathogen, such as SARS-CoV-2 or an influenza virus). A diagnostic system, as described herein, may be self-administrable and comprise a sample-collecting component (e.g., a swab) and a diagnostic device. The diagnostic device may comprise a cartridge, a blister pack, and/or a “chimney” detection component, according to some embodiments. In some cases, the diagnostic device comprises a detection component (e.g., a lateral flow assay strip, a colorimetric assay), results of which are self-readable, or automatically read by a computer algorithm. In certain embodiments, the diagnostic device further comprises one or more reagents (e.g., lysis reagents, nucleic acid amplification reagents, CRISPR/Cas detection reagents). In certain other embodiments, the diagnostic system separately includes one or more reaction tubes comprising the one or more reagents. The diagnostic device may also comprise an integrated heater, or the diagnostic system may comprise a separate heater. The isothermal amplification technique employed yields not only fast but very accurate results.

According to exemplary embodiments described herein, it may be desirable to selectively move solutions contained in reservoirs of a diagnostic test component, such as a detection component. In particular, the inventors have recognized that moving reagents through a diagnostic test at specific times in a sterile manner may provide accurate, easy to perceive results in a rapid manner. The inventors have also recognized the benefits of one or more reservoirs with corresponding seals configured to ensure that at certain times, fluids are maintained separate from one another (and other parts of a device) when the seals are closed, and at other times allow fluid transmission and/or mixing when the seals are opened. Accordingly, the inventors have recognized the benefits of one or more seals positioned between one or more reservoirs of a diagnostic test and a lateral flow assay strip, where the reservoirs may be a part of a cartridge, blister pack, and/or a “chimney” detection component. In some embodiments, the one or more seals may be configured as puncturable seals. Puncturable seals may be formed in a film composed of metal, plastic and/or elastomer. According to exemplary embodiments described herein, seals may be positioned between one or more reservoirs and a lateral flow assay strip. Additionally, seals of a diagnostic test may take different forms and may be used in any suitable combination with one another for single or multiple reservoirs.

In some embodiments, testing a sample on a lateral flow assay strip may generate one or more signal bands that indicate whether a target nucleic acid sequence is present in the sample. In some cases, the brightness of these signal bands may be at least partly determined based on how much a sample is diluted before being passed through the lateral flow assay strip. If a sample is not diluted prior to passing through the lateral flow assay strip, the signal bands may be dim and accordingly hard to perceive for an at-home user of a diagnostic test. Accordingly, the inventors have recognized the benefits of a detection component that includes a diluent reservoir containing a diluent that is configured to release the diluent to mix with a sample before that sample flows to a lateral flow assay strip. In some embodiments, the diluent reservoir may be separated from the lateral flow assay strip by a seal. When a reservoir containing a sample (e.g., a vial) is fluidly connected with the lateral flow assay strip, the seal may be opened to allow the diluent to mix with the sample as the sample flows toward the lateral flow assay strip. In other embodiments, a vial may include a first reservoir for containing a sample, and a second reservoir for containing a diluent solution. A seal between the first reservoir and second reservoir may be broken to allow the sample and the diluent solution to mix prior to the mixed sample and diluent solution flowing to a lateral flow assay strip. Other arrangements are also contemplated, as will be discussed further herein.

In some cases, it may be desirable to provide a detection component separate from other portions of a diagnostic test. For example, various components to take a sample and prepare the sample for detection in the detection component may be separate from the detection component. Accordingly, the inventors have recognized the benefits of a detection component including one or more fluid reservoirs containing one or more reagents configured to be employed in a detection process. In some embodiments, a detection component may include a lateral flow assay strip configured to receive a prepared sample to determine if one or more target nucleic acid sequences are present in the prepared sample. In some cases, as noted above, it may be desirable for a prepared sample to be mixed with a diluent prior to flowing through the lateral flow assay strip. Accordingly, in some embodiments, a detection component may include a reservoir containing a diluent configured to mix with a prepared sample contained in an external reservoir. The reservoir may include a seal, such as a puncturable seal, that fluidly separates the diluent reservoir from a lateral flow assay strip and/or a sample reservoir. In some embodiments, the act of fluidly connecting the sample reservoir to the lateral flow assay strip may open the seal of the diluent reservoir, thereby causing the diluent and sample to mix prior to flowing to the later flow assay strip. Various other embodiments will be described herein which promote the mixing of a sample and diluent in a manner that is easy to operate and sterile for an at-home user of a diagnostic system. Of course, a detection component may include any suitable reservoir containing any desired reagent for a detection process, as the present disclosure is not so limited.

In some embodiments, a detection component of a diagnostic test includes a first reservoir, a lateral flow assay strip, and a seal positioned between the first reservoir and the lateral flow assay strip. The seal may be configured to selectively allow a first solution contained in the first reservoir to flow from the first reservoir to the lateral flow assay strip. In some embodiments, the first reservoir, lateral flow assay strip, and seal may be disposed in a housing. In such an embodiment, the detection component may include a receptacle configured to receive a second reservoir (e.g., a vial) containing a sample. The first reservoir and receptacle may be configured so that when the second reservoir is moved into the receptacle, the seal may be opened to allow the first solution to flow toward the lateral flow assay strip. In this manner, the movement of the second reservoir into the receptacle may function as a trigger to release the first solution from the first reservoir. In some embodiments, the receptacle is configured to fluidly connect the second reservoir to the lateral flow assay strip. Accordingly, in a single action of inserting the second reservoir into the receptacle, a user may fluidly connect the first reservoir to the lateral flow assay strip and fluidly connect the second reservoir to the lateral flow assay strip. In some embodiments, the seal may be opened by the insertion of the second reservoir into the receptacle before the second reservoir is fluidly connected to the lateral flow assay strip. In other embodiments, the seal may be opened concurrently with the second reservoir being fluidly connected to the lateral flow assay strip. In still other embodiments, the seal may be opened after the second reservoir is fluidly connected to the lateral flow assay strip. Of course, any suitable timing for opening the seal of the first reservoir may be employed, as the present disclosure is not so limited.

According to exemplary embodiments described herein, a seal of a detection component may be formed of a frangible material, such that the seal may be punctured or otherwise destructively broken to be opened. For example, a puncturable seal may be a breakable metal foil, a breakable film such as a plastic film or an elastomeric film that is puncturable. In some embodiments, the seal may be positioned in a fluid channel between a first reservoir containing a first solution and a lateral flow assay strip. According to one such embodiment, insertion of a second reservoir (e.g., a vial) containing a sample into a receptacle of the detection component may puncture the seal to release the first solution to flow toward the lateral flow assay strip. For example, in some embodiments, the insertion of the second reservoir may crush the seal. As another example, the insertion of the second reservoir into the receptacle may pressurize the first reservoir until a threshold pressure is reached, whereupon the seal is punctured by the pressure. In some embodiments, the second reservoir may interact with an actuator when the second reservoir is inserted into the receptacle. For example, the second reservoir may depress a lever or plunger which may in turn open the seal. In some embodiments, the first reservoir may be formed as a blister, and the seal may be formed as a wall of the blister. In such an embodiment, the second reservoir may be configured to crush the blister to rupture the wall of the blister when the second reservoir is inserted into the receptacle. Of course, any suitable arrangement for a detection component including a seal may be employed, as the present disclosure is not so limited.

According to exemplary embodiments described herein, a seal of a detection component may be formed as a valve. The valve may be switched between a closed state where a solution in the first reservoir is not able to flow to the lateral flow assay strip and an open state where the solution is able to flow to the lateral flow assay strip. In some embodiments, movement of a second reservoir into a receptacle of the detection component may open the valve. In some embodiments, the valve may be a septum valve that is opened by the second reservoir when the second reservoir is moved into the receptacle. For example, the second reservoir may apply pressure to the first reservoir until a threshold pressure is reached, whereupon the septum valve may open to release the solution contained in the first reservoir. In other embodiments, the valve may be configured as a ball valve, flutter valve, umbrella valve, pinch valve, or any other suitable valve that may interact with a second reservoir as the second reservoir is moved into a receptacle. In some embodiments, a detection component may include an actuator coupled to the valve configured to open and/or close the valve when the second reservoir is inserted into a receptacle of the detection component. For example, the actuator may be a lever or a plunger that is moved by the second reservoir to switch the valve from a closed state to an open state. Of course, any suitable actuator may be used to apply pressure to a portion of a first reservoir of a detection component and/or open or close a valve, as the present disclosure is not so limited.

In some cases, it may be desirable to provide the fluid solutions for performing a diagnostic test in a single component for ease of manufacturing and use. For example, in some embodiments, a vial may include an internal volume having multiple fluid reservoirs that are configured to contain different solutions. In some embodiments, a vial may include a first reservoir configured to contain a sample, and a second reservoir configured to contain a first solution such as a diluent solution. The vial may also include a seal positioned between the first reservoir and second reservoir that may be opened to fluidly connect the first reservoir and the second reservoir. The seal may be opened by an external component to the vial, such that an external force may be employed to open the seal and fluidly connect the first reservoir and second reservoir. For example, in some embodiments, a detection component of a diagnostics test may include a housing containing lateral flow assay strip, and a receptacle configured to receive a vial including multiple reservoirs separated by one or more seals. The detection component may also include an actuator configured to apply a force to the one or more seals of the actuator to selectively open the seals to allow fluid to transfer between the reservoirs of the vial. In some embodiments, a single seal of the vial may be opened by a single actuator of the detection component. In other embodiments, multiple seals of the vial may be opened by a single actuator or multiple actuator. In an embodiment having multiple seals within a vial, the seals may be opened sequentially, so as to allow solutions to mix and transfer between reservoirs in a predetermined order. For example, in some embodiments a vial includes a first reservoir containing a sample, a second reservoir containing a first solution, and a third reservoir. The first reservoir and second reservoir may be separated by a first seal, and the second reservoir may be separated from the third reservoir by a second seal. The detection component may be configured to open the first seal first, to allow the sample and the first solution to mix inside of the first reservoir and/or second reservoir. Then, the second seal maybe opened to allow the mixed solution to flow to the third reservoir. In this manner, solutions may be mixed by a detection component before being transferred to another reservoir. In some embodiments, one or more of the reservoirs of a vial may be in fluid communication with the lateral flow assay strip.

According to some embodiments where a vial includes multiple reservoirs and one or more seals disposed between the multiple reservoirs, the one or more seals of the vial may be configured as any suitable seal that may be opened by an actuator of a detection component. In some embodiments, the one or more seals may be configured as frangible seals that make be broken by an actuator to open the seals. That is, the seals may be opened under direct force from the actuator, or from pressure applied to one of the multiple reservoirs. A frangible seal may be a metal foil, a plastic film, or an elastomeric film that is puncturable. In some embodiments, one or more of the multiple reservoirs may be formed as a blister formed in the vial. According to such an embodiment, a seal may be formed by a wall of the blister. In other embodiments, the one or more seals may be configured as valves that may be engaged by the actuator to switch the valves from a closed state to an open state. In some embodiments, one or more seals may be configured as a septum valve, where the septum valves are configured to open when an associated reservoir is pressurized to a threshold pressure. Of course, any suitable valve may be employed, as the present disclosure is not so limited.

According to exemplary embodiments described herein, a detection component of a diagnostic test may include one or more actuators configured to apply force to one or more seals to open the one or more seals. In some embodiments, the actuators may be unpowered, and may function to redirect forces from a user into force applied against the one or more seals. For example, an actuator configured as a lever may transfer linear force applied by a user to insert a vial into a receptacle into force applied against a seal. In other embodiments, an actuator may be powered, such that an actuator may open or more seals independent of force applied by a user. For example, an actuator may be coupled to a linear actuator, motor, servo, solenoid, or another suitable powered actuator that may apply force to one or more seals to open the one or more seals. According to such embodiments, an actuator may be controlled by a processor configured to execute instructions stored in volatile or non-volatile memory. In some embodiments, an actuator may be triggered to apply force to one or more seals by a condition that may be determined by the processor. For example, the processor may determine the presence of a vial in a receptacle of the detection component based on the input from one or more sensors (e.g., a button, switch, optical sensor, proximity sensor, etc.) and trigger the actuator to apply force to the one or more seals. Of course, any suitable actuator arrangement may be employed, as the present disclosure is not so limited.

While exemplary embodiments described herein relate to the release of a reservoir containing a diluent solution, it should be understood that the techniques described herein may be applied to any suitable solution. A reservoir associated with a seal that is opened upon the insertion of another reservoir into a receptacle may contain any desired solution including any number of reagents for a diagnostic test, as the present disclosure is not so limited.

As the COVID-19 pandemic has highlighted, there is a critical need for rapid, accurate systems and methods for diagnosing diseases-particularly infectious diseases. In the absence of diagnostic testing, asymptomatic infected individuals may unknowingly spread the disease to others, and symptomatic infected individuals may not receive appropriate treatment. With testing, however, infected individuals may take appropriate precautions (e.g., self-quarantine) to reduce the risk of infecting others and may receive targeted treatment as helpful.

While diagnostic tests for various diseases, including COVID-19, are known, such tests often require specialized knowledge of laboratory techniques and/or expensive laboratory equipment. For example, polymerase chain reaction (PCR) tests generally require skilled technicians and expensive, bulky thermocyclers. In addition, there is a need for diagnostic tests that are both rapid and highly accurate. Known diagnostic tests with high levels of accuracy often take hours, or even days, to return results, and more rapid tests generally have low levels of accuracy. Many rapid diagnostic tests detect antibodies, which generally can only reveal whether a person has previously had a disease, not whether the person has an active infection. In contrast, nucleic acid tests (i.e., tests that detect one or more target nucleic acid sequences) may indicate that a person has an active infection.

Diagnostic devices, systems, and methods described herein may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents are contained within a reaction tube, a cartridge, and/or a blister pack, such that users are not exposed to any potentially harmful chemicals.

Diagnostic devices, systems, and methods described herein are also highly sensitive and accurate. In some embodiments, the diagnostic devices, systems, and methods are configured to detect one or more target nucleic acid sequences using nucleic acid amplification (e.g., an isothermal nucleic acid amplification method). Through nucleic acid amplification, the diagnostic devices, systems, and methods are able to accurately detect the presence of extremely small amounts of a target nucleic acid. In certain cases, for example, the diagnostic devices, systems, and methods can detect 1 pM or less, or 10 aM or less.

As a result, the diagnostic devices, systems, and methods described herein may be useful in a wide variety of contexts. For example, in some cases, the diagnostic devices and systems may be available over the counter for use by consumers. In such cases, untrained consumers may be able to self-administer the diagnostic test (or administer the test to friends and family members) in their own homes (or any other location of their choosing). In some cases, the diagnostic devices, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic devices, systems, or methods may be operated or performed by the test subjects (e.g., students, teachers, patients, employees) or by designated individuals (e.g., a school nurse, a teacher, a school administrator, a receptionist).

In some embodiments, diagnostic devices described herein are relatively small. In certain cases, for example, a cartridge is approximately the size of a pen or a marker. Thus, unlike diagnostic tests that require bulky equipment, diagnostic devices and systems described herein may be easily transported and/or easily stored in homes, businesses, and medical points-of-care. In some embodiments, the diagnostic devices and systems are relatively inexpensive. Since no expensive laboratory equipment (e.g., a thermocycler) is required, diagnostic devices, systems, and methods described herein may be more cost effective than known diagnostic tests.

In some embodiments, any reagents contained within a diagnostic device or system described herein may be thermostabilized, and the diagnostic device or system may be shelf stable for a relatively long period of time. In certain embodiments, for example, the diagnostic device or system may be stored at room temperature (e.g., 20° C. to 25° C.) for a relatively long period of time (e.g., at least 1 month, at least 3 months, at least 6 months, at least 9 months, at least 1 year, at least 5 years, at least 10 years). In certain embodiments, the diagnostic device or system may be stored across a range of temperatures (e.g., 0° C. to 20° C., 0° C. to 37° C., 0° C. to 60° C., 0° C. to 90° C., 20° C. to 37° C., 20° C. to 60° C., 20° C. to 90° C., 37° C. to 60° C., 37° C. to 90° C., 60° C. to 90° C.) for a relatively long period of time (e.g., at least 1 month, at least 3 months, at least 6 months, at least 9 months, at least 1 year, at least 5 years, at least 10 years).

### Puncturable Blister in a Diagnostic Test Detection Component

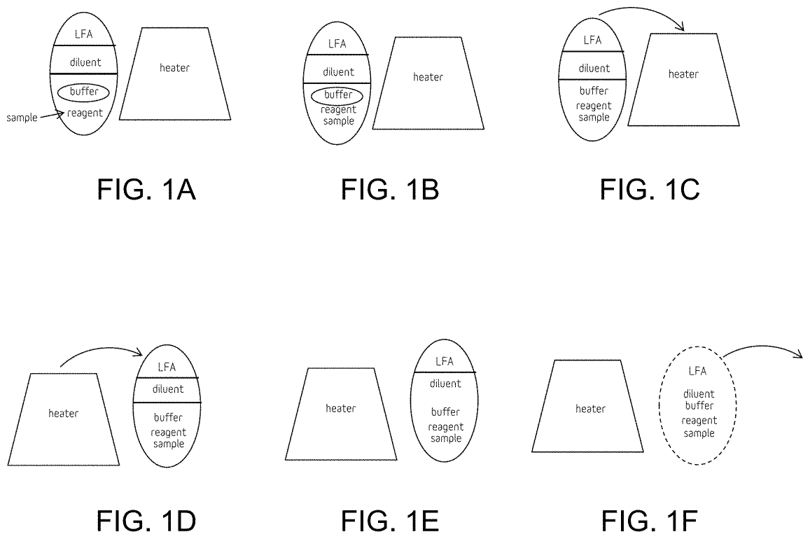

FIGS. 1A-1F show, according to some embodiments, a general process of performing a diagnostic test for the presence of one or more nucleic acid sequences. As shown in FIG. 1A, a sample is added to a first reservoir containing one or more reagents (e.g., lysis reagents, nucleic acid amplification reagents, CRISPR/Cas detection reagents). The one or reagents may react with the sample to begin a diagnostic testing process. As shown in FIG. 1A, a second reservoir containing a diluent may be kept separately from the first reservoir containing the reagents. As shown in FIG. 1B, the sample and reagent may mix for a predetermined time period. A buffer may also be combined into the first reservoir, such that a mixture of the sample, one or more reagents, and buffer are contained in the first reservoir. As shown in FIG. 1C, the mixture in the first reservoir may be heated in a heater or by another appropriate method such as an exothermic chemical reaction. Once heated as shown in FIGS. 1D-1E, the heated mixture contained in the first reservoir may be subsequently mixed with the diluent solution contained in the second reservoir. Once the diluent and sample mixture have mixed, the combined diluent and sample may be exposed to a lateral flow assay (LFA) strip, which may indicate the results of the diagnostic test. Following the reading of the LFA strip, one or more disposable components may be disposed of, as shown in FIG. 1F.

As shown in FIGS. 1A-1F, the process of performing a diagnostic test includes multiple steps of fluid combination at different times. Furthermore, additional steps such as heating are also performed through the testing process. According to exemplary embodiments described herein, the inventors have appreciated at least partially separating a detection component from other steps in the diagnostic testing process. That is, the steps shown in FIGS. 1D-1F may be accomplished using a detection component that simplifies the combination of a diluent and a sample mixture prior to being exposed to a lateral flow assay strip. Such arrangements may ensure that readings on a lateral flow assay strip are clear and well-defined, so that they may be easily perceived by an at-home user of a diagnostic testing system.

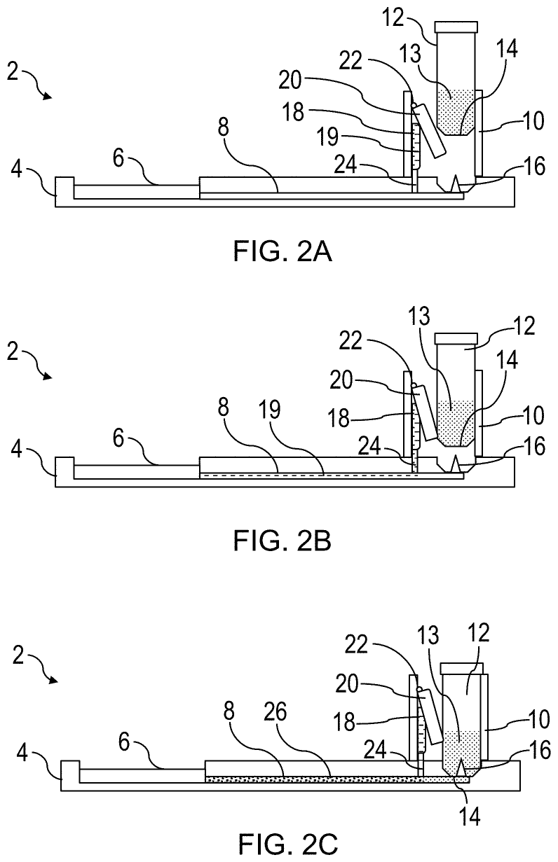

FIGS. 2A-2C show, according to some embodiments, a detection component 2 of a diagnostic test configured to ensure appropriate mixing of a sample mixture 13 and a diluent 19 prior to exposure to a lateral flow assay strip 6. As shown in FIG. 2A, the detection component includes a housing 4 containing the lateral flow assay strip 6 and a fluidic channel 8. The fluidic channel 8 fluidly connects the lateral flow assay strip 6 to a receptacle 10 and a first reservoir 18. According to the embodiment of FIGS. 2A-2C, the receptacle is configured to receive a second reservoir 12 (e.g., a vial) so that the second reservoir may be fluidly connected to the fluidic channel 8. The second reservoir 12 may be slidably disposed in the receptacle 10, such that the second reservoir may be moved into the receptacle to be punctured by a blade 16 to that the second reservoir may be brought into fluid communication with the fluidic channel 8. In particular, the blade 16 is configured to puncture a bottom portion 14 of the second reservoir to fluidly connect the second reservoir to the fluidic channel 8 and correspondingly allow the sample mixture 13 to flow into the fluidic channel toward the lateral flow assay strip. Of course, in other embodiments, a needle or another suitable puncturing component or other fluidic connection may be employed, as the present disclosure is not so limited.

According to the embodiment of FIGS. 2A-2C, the detection component 2 includes a first reservoir 18 containing a diluent 19. As noted previously, before the sample mixture 13 is bought into fluid communication with the lateral flow assay strip, it is desirable to sufficiently dilute the sample mixture to ensure the readout from the lateral flow assay strip is clear. Accordingly, the detection component 2 of FIGS. 2A-2C is configured to release the diluent 19 into fluid communication with fluidic channel 8 concurrently or before the second reservoir 12 is fluidly connected to the fluidic channel. As shown in FIG. 2A, the detection component includes a seal 24 positioned between the first reservoir 18 and the fluidic channel 8. According to the embodiment of FIGS. 2A-2C, the seal 24 is a frangible seal that is configured to open once a threshold pressure is reached inside of the first reservoir 18. As will be discussed further with reference to FIGS. 2B and 2C, the insertion of the second reservoir 12 into the receptacle 10 applies pressure to the first reservoir 18, thereby breaking the seal 24 and allowing the diluent to flow into the fluidic channel 8. According to the embodiment of FIGS. 2A-2C, the detection component 2 includes an actuator 20 configured to assist in transferring force applied to the second reservoir 12 (e.g., when inserting the second reservoir into the receptacle 10) into force applied to the first reservoir 18. As shown in FIG. 2A, the actuator 20 functions as a second-class lever. The actuator 20 is configured to rotate about actuator pin 22, where insertion of the second reservoir into the receptacle 10 rotates the actuator into contact with the first reservoir.

The process of using the detection component 2 to perform a diagnostic test is shown through the states of FIGS. 2A-2B. FIG. 2A is a starting state where the second reservoir 12 is not fluidly connected to the reservoir 18. Correspondingly, the seal 24 is closed and accordingly the diluent 19 is contained within the first reservoir 18. The actuator 20 is in a first rotational position, where force is not being applied to the first reservoir by the actuator. Accordingly, the pressure of the first reservoir is below a threshold pressure, and the seal 24 remains closed. The fluidic channel 8 is empty in the state of FIG. 2A. In the state of FIG. 2B, the second reservoir 12 has been pushed further into the receptacle 10 toward the blade 16. Accordingly, the bottom portion 14 of the second reservoir is engaged with the actuator 20, which has rotated to a second rotational position about the actuator pin 22 where the actuator is closer to the first reservoir 18. Force has been transmitted from the second reservoir 12 to the first reservoir 18, such that the pressure of the first reservoir has exceed a threshold value. Accordingly, the seal 24 has opened, allowing the diluent 19 to fill the fluidic channel 8. However, the sample mixture 13 is still contained in the second reservoir 12, as the bottom portion 14 has not be punctured by the blade 16. Thus, diluent is present in the fluidic channel 8 in fluid communication with the lateral flow assay strip 6 prior to the sample mixture 13.

As shown in FIG. 2C, continued insertion of the second reservoir 12 into the receptacle punctures the second reservoir with the blade 16. That is, the bottom portion 14 of the second reservoir is punctured to allow the sample mixture 13 to flow into the fluidic channel 8 and mix with the diluent to form a sample diluent mixture 26 that in turn flows to the lateral flow assay strip 6. As noted previously, diluent 19 is present in the fluidic channel 8 in fluid communication with the lateral flow assay strip 6 prior to the sample mixture 13. Such an arrangement ensures that a concentration of the diluent relative to the sample is higher when the sample first encounters the lateral flow assay strip 6. The higher concentration of diluent initially may assist the lateral flow assay strip is generating definitive signal lines that may be easily perceived by a user. Of course, in other embodiments the diluent 19 may evenly mix with the sample mixture 13 before encountering the lateral flow assay strip, as the present disclosure is not so limited.

While in the embodiment of FIGS. 2A-2C the fluidic channel 8 is in direct fluid communication with the lateral flow assay strip 6, in other embodiments the detection component may include a sample pad joined to the lateral flow assay strip 6, which functions as an input for the lateral flow assay strip. Of course, any suitable arrangement for a lateral flow assay strip may be employed as the present disclosure is not so limited.

According to the embodiment of FIGS. 2A-2C, the second reservoir 12 is configured to contain a sample. In some embodiments, the second reservoir may be configured to receive a sample swab (not shown), where the swab may be configured to collect a sample from a subject. The second reservoir 12 may contain a buffer solution and/or a lysis solution configured to react with the sample so that one or more target nucleic acid sequences may be detected by the lateral flow assay strip. The second reservoir 12 may be sized and shaped to fully receive the swab. Accordingly, a sample swab may be easily deposited in the reservoir 12, and once the swab is deposited in the reservoir 12, the reservoir may be sealed with a cap and allowed to incubate before the bottom portion 14 of the second reservoir 12 is punctured. In some embodiments, the second reservoir may be formed as a plastic vial. Of course, the second reservoir may have any suitable construction, as the present disclosure is not so limited.

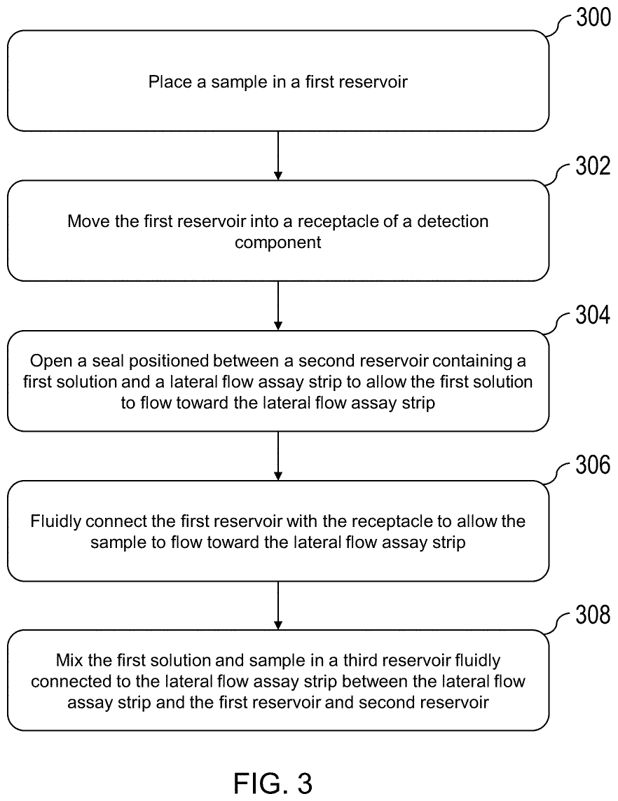

FIG. 3 shows, according to some embodiments, a flow chart for a method of performing a diagnostic test. In block 300, a sample is placed in a first reservoir, such as a vial. Placing the sample in the first reservoir may include placing a swab in the reservoir, in addition to one or more reagents. In other embodiments, a completed sample mixture may be placed in the first reservoir. In block 302, the first reservoir is moved into a receptacle of a detection component. For example, the first reservoir may be slid into a receptacle of the detection component. In block 304, a seal is opened between a second reservoir containing a first solution and a lateral flow assay strip to allow the first solution to flow toward the lateral flow assay strip. The first solution may be a diluent. In some embodiments, the force applied to the first reservoir moving the first reservoir into the receptacle may be directly applied to the seal to open the seal (e.g., by crushing or puncturing). In other embodiments, the force applied to the first reservoir moving the first reservoir into the receptacle may be applied to the second reservoir to pressurize the second reservoir. In some embodiments, the force applied to the first reservoir moving the first reservoir into the receptacle may be transferred to the second reservoir and/or the seal via an actuator, such as a plunger or lever.

As shown in FIG. 3, in block 306 the first reservoir is fluidly connected with the receptacle to allow the sample contained inside of the first reservoir to flow toward the lateral flow assay strip. Fluidly connecting the first reservoir to the receptacle may include puncturing the first receptacle with a puncturing tool, such as a blade or a needle. In other embodiments, the first reservoir may be fluidly connected to the receptacle using a fluid connector (e.g., a quick connect fluid connector). In block 308, the first solution from the second reservoir and the sample are mixed in a third reservoir fluidly connected to the lateral flow assay strip, where the third reservoir is positioned between the lateral flow assay strip and the first reservoir and second reservoir. In some embodiments, the third reservoir may be a fluidic channel disposed between the lateral flow assay strip and the first reservoir and second reservoir.

### Detection Component Including Internal Actuators

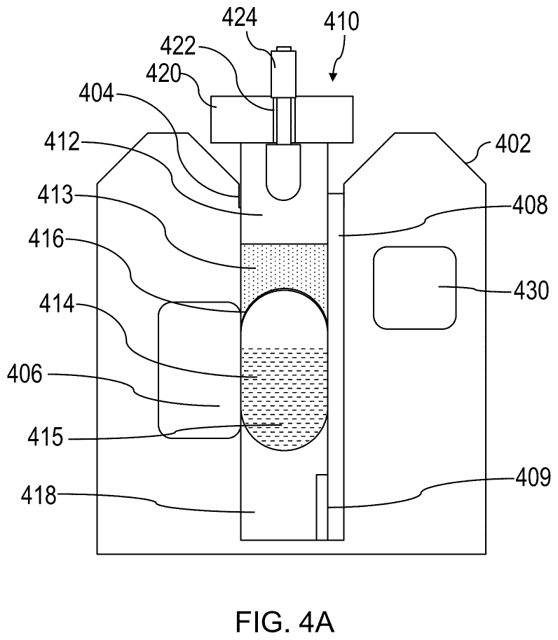

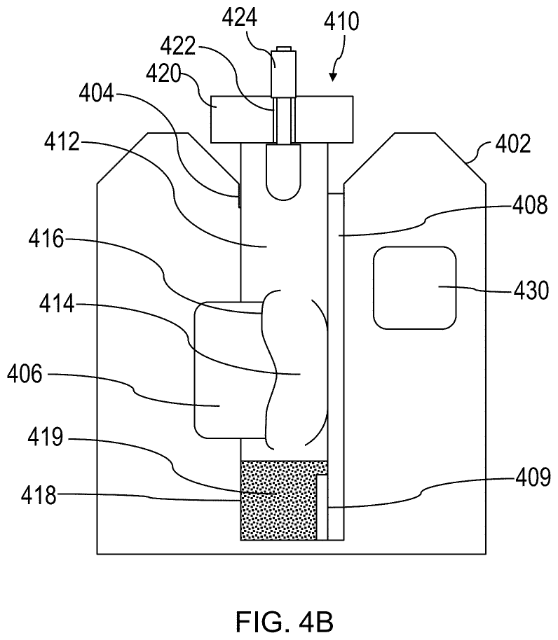

As discussed previously, in some embodiments a diagnostic device includes a detection component including one or more actuators configured to selectively open one or more seals between reservoirs to allow fluid transfer between said reservoirs. By controlling the actuators to selectively open one or more seals, various fluid volumes of a diagnostic test may be selectively combined at a desired time. For example, in some embodiments, a detection component may be configured to selectively open a seal between a first reservoir containing a sample and a second reservoir containing a diluent. In such an embodiment, the opening of the seal may allow the sample to mix with the diluent prior to flowing to a lateral flow assay strip. As described herein, the lateral flow assay strip may comprise one or more test lines configured to detect one or more target nucleic acid sequences. In some embodiments, the lateral flow assay strip further comprises one or more control lines. Arrangements of various detection components described herein may ensure that a sample is appropriately diluted, so that a signal readout (e.g., test line) on the lateral flow assay strip is clearly defined to a user or a reader. As will be discussed further with reference to the embodiments of FIGS. 4A-8, detection components may include one or multiple actuators configured to open any suitable number of seals in a diagnostic test to facilitate the detection of one or more target nucleic acid sequences.

FIGS. 4A-4B show a detection component of a diagnostic device including an actuator 406. As shown in FIG. 4A, the detection component includes a housing 402 including a receptacle 404 configured to receive a vial 410. The detection component includes an actuator disposed in the housing 402 and configured to selectively move into the receptacle 404 to engage the vial 410. The detection component also includes a lateral flow assay strip 408 having a sample pad 409 configured as an input to the lateral flow assay strip. According to the embodiment of FIGS. 4A-4B, the lateral flow assay strip 408 is positioned inside of the housing 402, so that the lateral flow assay strip is not visible externally to a user of the detection component. Instead, in some embodiments as shown in FIG. 4A, the detection component includes a reader 430 configured to sense the presence of one or more test lines on the lateral flow assay strip. The reader 430 may be an optical sensor or another suitable sensor configured to read an output of the lateral flow assay strip. In some embodiments, the reader 430 may transmit a reading from the lateral flow assay strip to a processor that may be positioned in the housing 402 or on a remote device (e.g., a mobile device such as a smartphone).

According to the embodiment of FIGS. 4A-4B, the detection component is configured to receive a vial 410 including multiple reservoirs. As shown in FIG. 4A, the vial 410 includes a first reservoir 412 configured to contain a sample mixture 413. In some embodiments as shown in FIG. 4A, the vial 410 includes a cap 420 including a port 422 through which a sample swab 424 is received. The sample swab may contact one or more reagents contained within the first reservoir 412 to form the sample mixture 413. Of course, other arrangements are contemplated, including arrangements where a swab 424 is not inserted into the first reservoir, as the present disclosure is not so limited. In one such embodiment, the sample mixture 413 may be transferred from another container (e.g., with a pipette or other suitable tool).

As shown in FIG. 4A, the vial 410 also includes a second reservoir 414 containing a first solution 415. In some embodiments, the first solution is a diluent solution configured to dilute the sample mixture 413 before the sample mixture 413 is exposed to the lateral flow assay strip 408. In the embodiment of FIGS. 4A-4B, the second reservoir is configured as a blister or ampoule formed inside of the vial 410. That is, a seal 416 configured as a wall of the blister separates the first reservoir 412 from the second reservoir 414. The seal is configured as a frangible seal, such that the seal may be broken to fluidly connect the first reservoir 412 to the second reservoir 414. As shown in FIG. 4A, the vial 410 also includes a third reservoir 418 in fluid communication with the sample pad 409. In some embodiments, the third reservoir may include a hole configured to align with the sample pad 409 disposed in the housing 402 of the detection component. In other embodiments, any suitable fluid connection between the third reservoir and the sample pad 409 may be employed, as the present disclosure is not so limited. According to the embodiment of FIGS. 4A-4B, the seal 416 also separates the first reservoir 412 and second reservoir 414 from the third reservoir 418 until the seal is opened. Accordingly, in the state shown in FIG. 4A, the first reservoir 412, second reservoir 414, and third reservoir 418 are all fluidly isolated from one another by the seal 416.

According to the embodiment of FIGS. 4A-4B, the seal 416 is configured to be opened via actuation of the actuator 406 in the housing. That is, the actuator 406 is configured to be moved from a first position to a second position, where in the second position the actuator opens the seal 416. The actuator 406 is a crush effector configured to apply force to the seal 416 as the actuator moves from the first position to the second position to rupture the seal 416. In particular, the actuator is configured to apply force to the second reservoir 414 to increase the pressure inside of the second reservoir until the pressure reaches a threshold value, whereupon the seal 416 ruptures to fluidly connect the first reservoir 412, second reservoir 414, and third reservoir 418. According to the embodiment of FIGS. 4A-4B, a wall of the vial 410 may be flexible to accommodate deformation under force of the actuator 406. In other embodiments, the actuator 406 may engage the seal 416 through an opening formed in a side of the vial 410. In some embodiments the actuator 406 may be moved between the first position and the second position by a motor, linear actuator, or solenoid configured to apply an appropriate level of force to the seal 416. In such an embodiment, the housing 402 may include a battery or other energy source configured to power the movement of the actuator 406 between the first position and second position. In other embodiments, the actuator 406 may be operatively connected to a handle, button, lever, or other suitable human interface so that a user may apply force to the seal 416 via the actuator.

As shown in FIG. 4B, once the seal 416 is open, the sample and first solution mix in the third reservoir 418 to form a diluted sample 419. In the embodiment of FIGS. 4A-4B, the sample and diluent may flow to the third reservoir under the effect of gravity. As discussed previously, the third reservoir 418 is in fluid communication with the lateral flow assay strip 408 via the sample pad 409. Accordingly, the diluted sample 419 is tested on the lateral flow assay strip, where the results may be measured by the reader 430. Of course, in other embodiments the results of the lateral flow assay strip 408 may be visible and interpreted by a user, as the present disclosure is not so limited.

It should be noted that while a vial 410 including a cap 420 is employed in the embodiment of FIG. 4A-4B, any suitable container may be employed including any suitable number of reservoirs, as the present disclosure is not so limited.

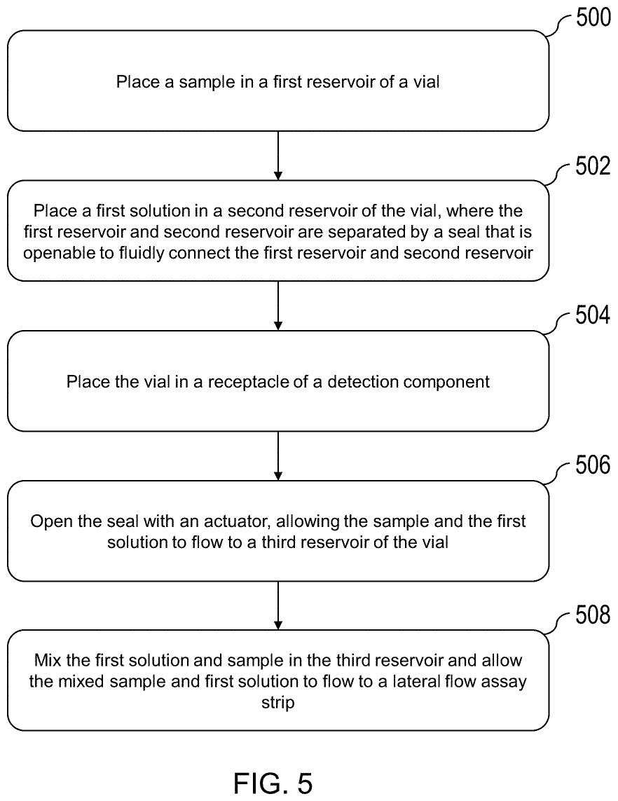

FIG. 5 shows, according to some embodiments, a flow chart for a method of performing a diagnostic test. In block 500, a sample is placed in a first reservoir of a vial. In block 502, a first solution is placed in a second reservoir of the vial, where the first reservoir and second reservoir are separated by a seal that is openable to fluidly connect the first reservoir and the second reservoir. In some embodiments, the seal may be a frangible seal configured to be opened by the application of direct force or indirect force (e.g., via pressurization of a reservoir). In one such embodiment, the seal may be formed as a wall of a blister. In block 504, the vial is placed in a receptacle of a detection component. In block 506, the seal is opened with an actuator, allowing the sample and the first solution to flow to a third reservoir of the vial. In some embodiments, opening the seal with the actuator may include moving the actuator from a first position to a second position. In the second position, the actuator may be at least partially disposed in the receptacle, so that the actuator is engaged with the seal or another portion of the vial. In some embodiments, the actuator may crush the seal to open the seal and fluidly connect the first reservoir and second reservoir. In block 508, the first solution and sample are mixed in the third reservoir. The mixed sample is allowed to flow to a lateral flow assay strip (e.g., via a sample pad) so that the mixed sample is tested on the lateral flow assay strip.

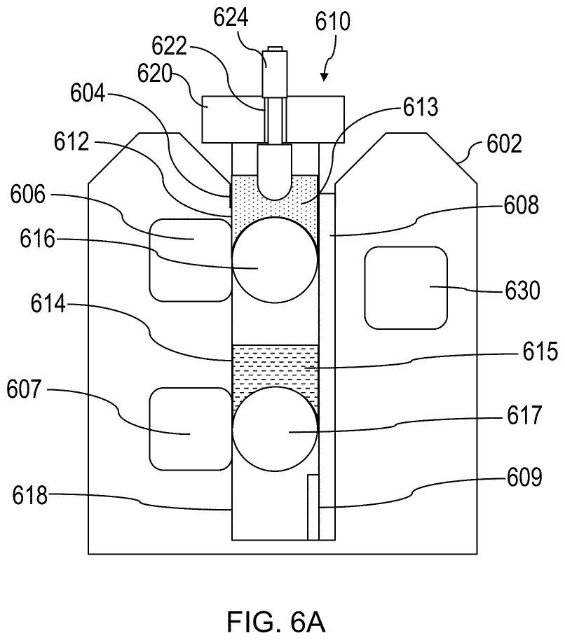

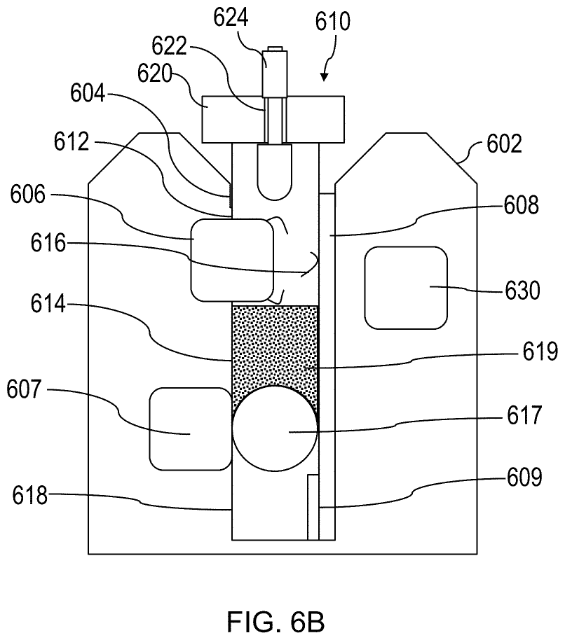

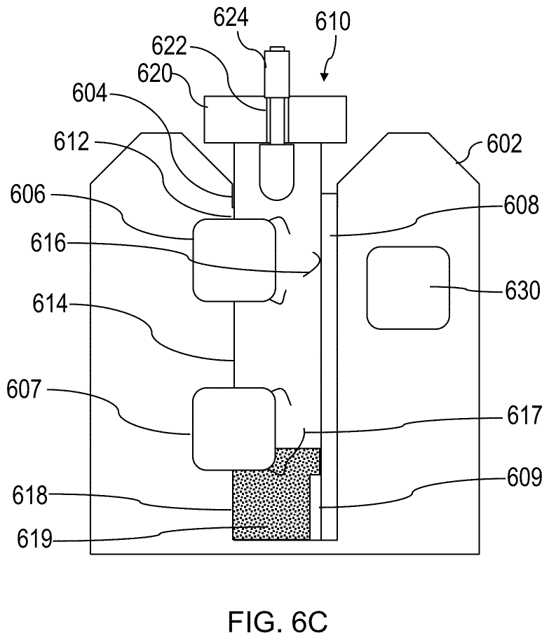

FIGS. 6A-6C show a detection component of a diagnostic device including a plurality of actuators 606, 607. As shown in FIG. 6A, the detection component includes a housing 602 including a receptacle 604 configured to receive a vial 610. The detection component includes a first actuator 606 and a second actuator 607 disposed in the housing 602, which are configured to selectively move into the receptacle 604 to engage the vial 610. The detection component also includes a lateral flow assay strip 608 having a sample pad 609 configured as an input to the lateral flow assay strip. According to the embodiment of FIGS. 6A-6C, the lateral flow assay strip 608 is positioned inside of the housing 602, so that the lateral flow assay strip is not visible externally to a user of the detection component. Instead, in some embodiments as shown in FIG. 6A, the detection component includes a reader 630 configured to sense the presence of one or more test lines on the lateral flow assay strip. The reader 630 may be an optical sensor or another suitable sensor configured to read an output of the lateral flow assay strip. In some embodiments, the reader 630 may transmit a reading from the lateral flow assay strip to a processor that may be positioned in the housing 602 or on a remote device (e.g., a mobile device such as a smartphone).

According to the embodiment of FIGS. 6A-6C, the detection component is configured to receive a vial 610 including multiple reservoirs. As shown in FIG. 6A, the vial 610 includes a first reservoir 612 configured to contain a sample mixture 613. In some embodiments as shown in FIG. 6A, the vial 610 includes a cap 620 including a port 622 through which a sample swab 624 is received. The sample swab may contact one or more reagents contained within the first reservoir 612 to form the sample mixture 613. Of course, other arrangements are contemplated, including arrangements where a swab 624 is not inserted into the first reservoir, as the present disclosure is not so limited. In one such embodiment, the sample mixture 613 may be transferred from another container (e.g., with a pipette or other suitable tool).

As shown in FIG. 6A, the vial 610 also includes a second reservoir 614 containing a first solution 615. In the embodiment of FIGS. 6A-6C, the first solution is a diluent solution configured to dilute the sample mixture 613 before the sample is exposed to the lateral flow assay strip 608. In the embodiment of FIGS. 6A-6C, the vial 610 includes a first seal 616 configured as a frangible seal that separates the first reservoir 612 from the second reservoir 614. The first seal may be broken to fluidly connect the first reservoir 612 to the second reservoir 614. As shown in FIG. 6A, the vial 610 also includes a third reservoir 618 in fluid communication with the sample pad 609. In some embodiments, the third reservoir may include a hole configured to align with the sample pad 609 disposed in the housing 602 of the detection component. In other embodiments, any suitable fluid connection between the third reservoir and the sample pad 609 may be employed, as the present disclosure is not so limited. According to the embodiment of FIGS. 6A-6C, the detection component includes a second seal 617 that separates the second reservoir 614 from the third reservoir 618 until the second seal is opened. Accordingly, in the state shown in FIG. 6A, the first reservoir 612, second reservoir 614, and third reservoir 618 are all fluidly isolated from one another by the first seal 616 and the second seal 617.

According to the embodiment of FIGS. 6A-6C, the first seal 616 is configured to be opened via actuation of the first actuator 606 in the housing 602. Likewise, the second seal 617 is configured to be opened via actuation of the second actuator 607 in the housing. That is, the first actuator 606 is configured to be moved from a first position to a second position, where in the second position the first actuator opens the first seal 616. Similarly, the second actuator 607 is configured to be moved from a first position to a second position, wherein in the second position the second actuator opens the second seal 617. The first actuator 606 is configured to apply force to the first seal 616 as the first actuator moves from the first position to the second position to rupture the seal 616. Likewise, the second actuator 607 is configured to apply force to the second seal 617 as the second actuator moves from the first position to the second position to rupture the second seal. In the embodiment of FIGS. 6A-6C, the first actuator 606 and the second actuator 607 are configured to move independently from one another between the first positions and the second positions. In other embodiments, the first actuator 606 and the second actuator 607 may be operatively coupled, so that movement of the first actuator may correspondingly move the second actuator. In some embodiments, the first actuator and second actuator may be configured to move sequentially (e.g., the first actuator may be moved to the second position, followed by the second actuator). Of course, any suitable arrangement of the first actuator and second actuator may be employed, as the present disclosure is not so limited.

According to the embodiment of FIGS. 6A-6C, a wall of the vial 610 may be flexible to accommodate deformation under force of the first actuator 606 and second actuator 607. In other embodiments, the first actuator 606 and second actuator 607 may engage the first seal 616 and second seal 617, respectively, through one or more openings formed in a side of the vial 610. In some embodiments, the first actuator 606 and the second actuator 607 may be moved between the first position and the second position by a motor, linear actuator, or solenoid configured to apply an appropriate level of force to the first seal 616 and the second seal 617. In such an embodiment, the housing 602 may include a battery or other energy source configured to power the movement of the first actuator 606 and second actuator 607 between the first positions and second positions. In other embodiments, the first actuator 606 and second actuator 607 may be operatively connected to a handle, button, lever, or other suitable human interface so that a user may apply force to the first seal 616 and the second seal 617 via the actuators.

As shown in FIG. 6B, once the first seal 616 is open, the sample and first solution mix in the second reservoir 614 to form a diluted sample 619. In the embodiment of FIGS. 6A-6C, the sample and diluent may flow to the second reservoir under the effect of gravity. Once the sample and first solution are appropriately mixed, the second seal 617 may be opened to allow the diluted sample 619 to flow to the third reservoir 618 (e.g., under the effect of gravity), as shown in FIG. 6C. As discussed previously, the third reservoir 618 is in fluid communication with the lateral flow assay strip 608 via the sample pad 609. Accordingly, the diluted sample 619 is tested on the lateral flow assay strip, where the results may be measured by the reader 630. Of course, in other embodiments the results of the lateral flow assay strip 608 may be visible and interpreted by a user, as the present disclosure is not so limited.

It should be noted that while a vial 610 including a cap 620 is employed in the embodiment of FIG. 6A-6C, any suitable container may be employed including any suitable number of reservoirs, as the present disclosure is not so limited.

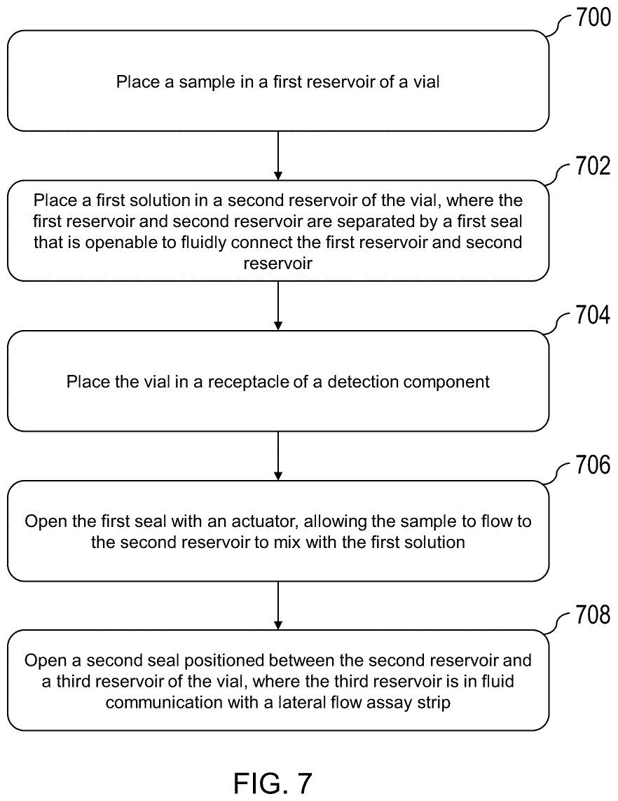

FIG. 7 shows, according to some embodiments, a flow chart for a method of performing a diagnostic test. In block 700, a sample is placed in a first reservoir of a vial. In block 702, a first solution is placed in a second reservoir of the vial. The first reservoir and the second reservoir rare separated by a first seal that is openable to fluidly connect the first reservoir and the second reservoir. The first seal may be a frangible seal such as a metal foil, plastic film, or an elastomeric film. In block 704, the vial is placed in a receptacle of the detection component. In block 706, the first seal is opened with an actuator, allowing the sample to flow to the second reservoir to mix with the first solution. In some embodiments, to open the first seal the actuator may engage the first seal to crush the first seal or otherwise apply a threshold force to open the first seal. In block 708, a second seal positioned between the second reservoir and a third reservoir of the vial is opened. Opening the second seal allows the mixed sample and first solution to flow the third reservoir. The third reservoir is ins fluid communication with a lateral flow assay strip, so that the mixed sample and first solution is tested on the lateral flow assay strip. In some embodiments, the actuator that opened the first seal may also open the second seal. In other embodiments the second seal may be opened by a second actuator that moves independently from the actuator.

### Method of Making a Diagnostic Test Including a Detection Component

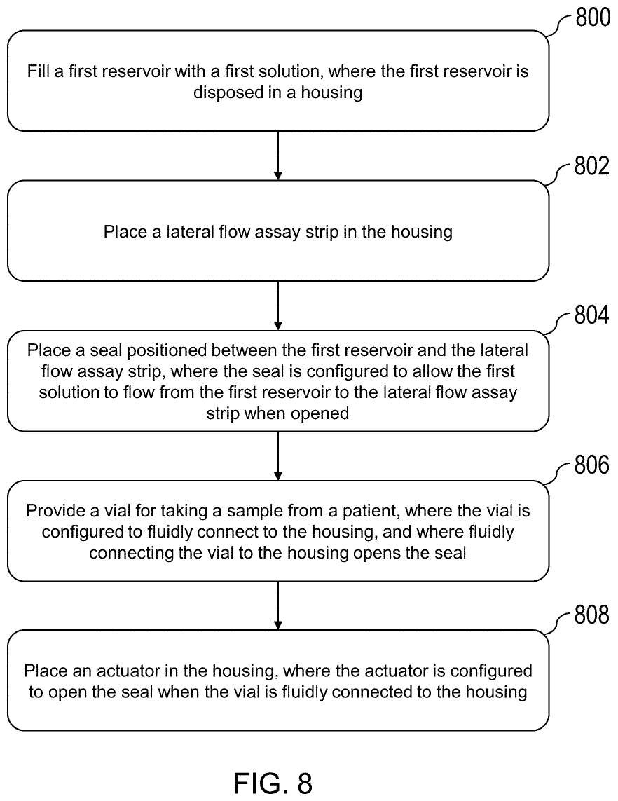

FIG. 8 shows, according to some embodiments, a flow chart for a method of making a diagnostic test. In block 800, a first reservoir is filled with a first solution, where the first reservoir is disposed in a housing. In block 802, a lateral flow assay strip is placed in the housing. In block 804, a seal is positioned between the first reservoir and the lateral flow assay strip. The seal is configured to allow the first solution to flow from the first reservoir to the lateral flow assay strip when opening. The seal may be a frangible seal, such as a metal foil, elastomeric film, or another suitable arrangement that is destructively opened. In block 806, a vial is provided for taking a sample from a patient. The vial may be configured to fluidly connect to the housing (e.g., via a receptacle). The action of fluidly connecting the vial to the housing opens the seal. For example, moving the vial to fluidly connect the vial to the housing may cause the vial to engage and open the seal. In block 808, an actuator is placed in the housing, where the actuator is configured to open the seal when the vial is fluidly connected to the housing. The process shown in FIG. 8 may be employed to make a diagnostic test similar to that shown and described with reference to FIGS. 2A-2C.

### “Chimney” Detection Component

In some embodiments, a diagnostic device comprises a detection component comprising a “chimney.” In certain embodiments, the “chimney” detection component comprises a chimney configured to receive a reaction tube. In certain embodiments, the “chimney” detection component comprises a puncturing component configured to puncture the reaction tube. The puncturing component may comprise one or more blades, needles, or other elements capable of puncturing a reaction tube. In certain embodiments, the “chimney” detection component comprises a lateral flow assay strip. As described herein, the lateral flow assay strip may comprise one or more test lines configured to detect one or more target nucleic acid sequences. In some embodiments, the lateral flow assay strip further comprises one or more control lines.

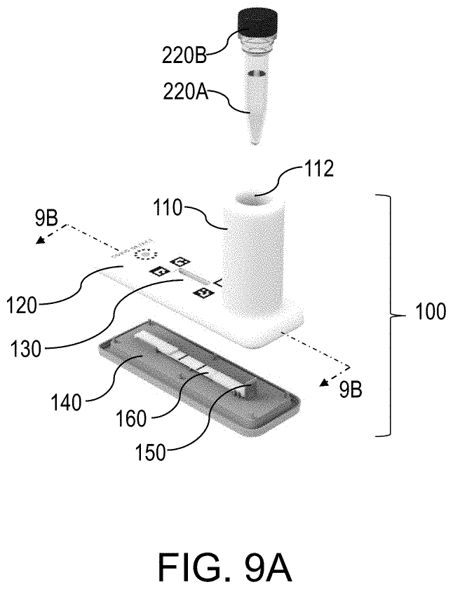

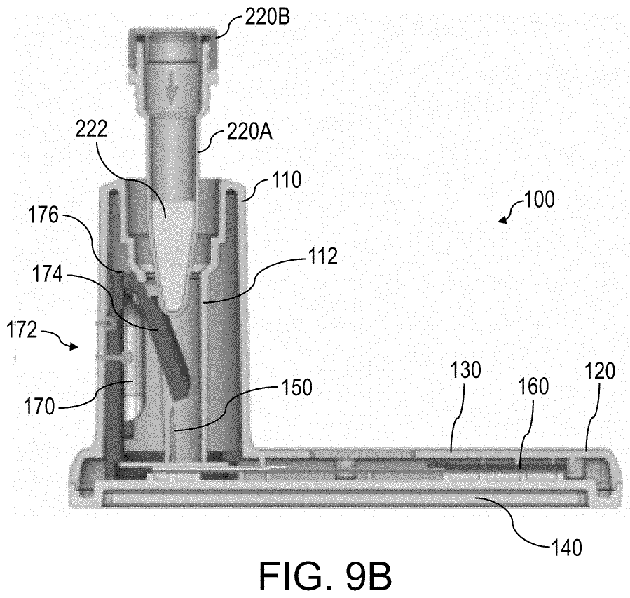

One embodiment of a “chimney” detection component is shown in FIG. 9A. In FIG. 9A, detection component 100 comprises chimney 110, front panel 120 comprising opening 130, and back panel 140 comprising puncturing component 150 and lateral flow assay strip 160. In some embodiments, chimney 110 and front panel 120 are integrally formed. In some embodiments, chimney 110 and front panel 120 are separately formed components that are attached to each other (e.g., via one or more screws or other fasteners, one or more adhesives, and/or one or more interlocking components). In some embodiments, front panel 120 and back panel 140 are attached to each other (e.g., via one or more screws or other fasteners, one or more adhesives, and/or one or more interlocking components). In some embodiments, front panel 120 comprises one or more markings (e.g., ArUco markers) to facilitate alignment of an electronic device (e.g., a smartphone, a tablet) with opening 130.

In operation, a reaction tube comprising fluidic contents may be inserted into chimney 110. In some embodiments, the reaction tube comprises a cap (e.g., a screw-top cap, a hinged cap) and a bottom end (e.g., a tapered or rounded bottom end). In certain cases, as shown in FIG. 9A, the bottom end of the reaction tube is inserted into chimney 110 prior to the cap of the reaction tube. In certain cases, the reaction tube is inverted, and the cap of the reaction tube is inserted into chimney 110 prior to the bottom end of the reaction tube. In some embodiments, upon insertion into chimney 110, the reaction tube may lock or snap into place (or may otherwise have a secure fit) such that the reaction tube may not be easily removed from chimney 110 by the user. In certain cases, locking or snapping the reaction tube into place (or otherwise preventing easy removal of the reaction tube from chimney 110) may reduce or prevent contamination.

In some embodiments, the reaction tube may be punctured by puncturing component 150. As a result, at least a portion of the fluidic contents of the reaction tube may be deposited on a first sub-region (e.g., a sample pad) of lateral flow assay strip 160. In some cases, at least a portion of the fluidic contents of the reaction tube may be transported through lateral flow assay strip 160 (e.g., via capillary action). In some cases, for example, at least a portion of the fluidic contents of the reaction tube may flow through a second sub-region (e.g., a particle conjugate pad) of lateral flow assay strip 160 comprising a plurality of labeled particles. In some instances, the fluidic contents of the reaction tube may comprise one or more amplified nucleic acids (e.g., amplicons), and flow of at least a portion of the fluidic contents through the second sub-region (e.g., particle conjugate pad) of lateral flow assay strip 160 may result in one or more labeled amplicons. In some cases, at least a portion of the fluidic contents of the reaction tube (which may, in some instances, comprise one or more labeled amplicons) may flow through a third sub-region (e.g., a test pad) comprising one or more test lines comprising one or more capture reagents (e.g., immobilized antibodies) configured to detect one or more target nucleic acid sequences. In some instances, the formation (or lack of formation) of one or more opaque lines may indicate the presence or absence of one or more target nucleic acid sequences. In certain cases, the one or more opaque lines (if present) may be visible through opening 130 of front panel 120.

FIG. 9B depicts a cross-sectional view of the detection component 100 showing additional components of the detection component. In particular, the cross-section of FIG. 9B depicts an internal reservoir 170 configured to contain a diluent solution. According to the embodiment of FIGS. 9A-9B, the detection component includes an actuator assembly 172 configured to allow the diluent in the internal reservoir to be released when the tube 220A is inserted into the chimney 110. As shown in FIG. 9B, the chimney 110 includes a receptacle 112 configured to receive the tube 220A. the actuator assembly 172 includes a lever 174 configured to rotate about a hinge 176. The tube 220A is configured to engage the lever 174 when the tube is inserted into the receptacle 112. In particular, as the tube 220A is moved into the receptacle, the lever 174 is rotated toward the reservoir 170 and applied force the reservoir. In the embodiment of FIG. 9B, the reservoir is configured as a blister, where application of a threshold force to the reservoir opens a seal and allow the diluent solution to flow toward the lateral flow assay strip 160 (e.g., by capillary action). Accordingly, as the tube 220A is inserted, force is applied to the reservoir 170 by the lever 174, releasing the diluent solution to the lateral flow assay strip. Afterwards, the tube is punctured by the puncturing component 150, allowing a sample 222 contained in the tube 220A to flow to the lateral flow assay strip. The diluent solution and sample 222 are allowed to mix, thereby improving the clarity of one or more test lines on the lateral flow assay strip.

In some embodiments, a diagnostic system comprises a sample-collecting component (e.g., a swab), a reaction tube comprising one or more reagents, and a “chimney” detection component. In some embodiments, the diagnostic system further comprises a heater, as described herein.

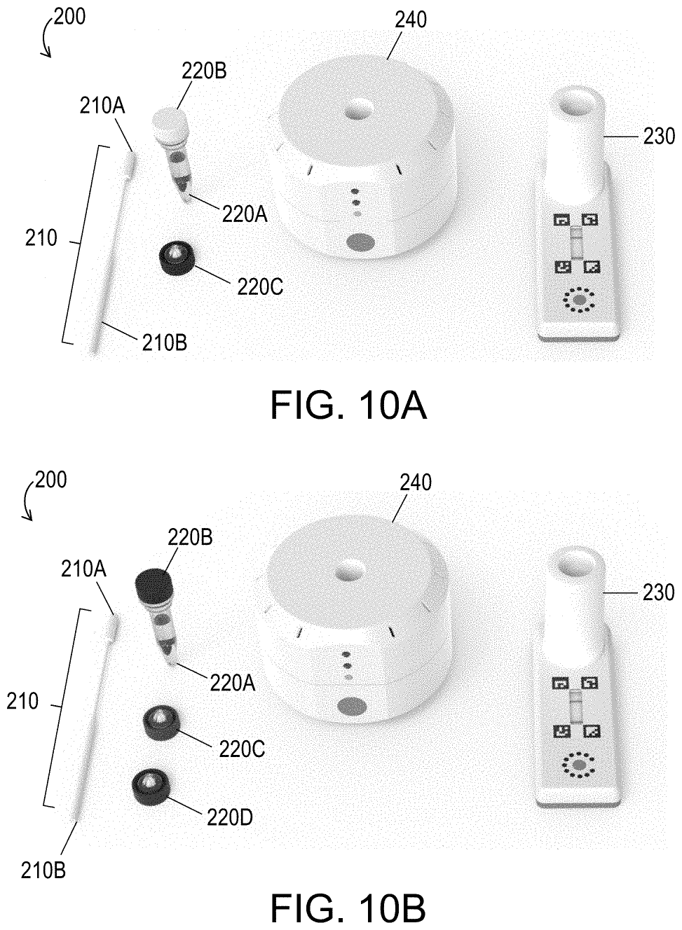

One embodiment of a diagnostic system comprising a “chimney” detection component is shown in FIG. 10A. In FIG. 10A, diagnostic system 200 comprises sample-collecting component 210, reaction tube 220, “chimney” detection component 230, and heater 240. As shown in FIG. 10A, sample-collecting component 210 may be a swab comprising swab element 210A and stem element 210B. In certain embodiment, reaction tube 220 comprises tube 220A, first cap 220B, and second cap 220C. As shown in FIG. 10A, first cap 220B and/or second cap 220C may be screw-top caps or any other types of removable caps. In certain embodiments, first cap 220B and/or second cap 220C may be airtight caps (e.g., they may fit on reaction tube 220A without any gaps and seal reaction tube 220A). In certain embodiments, second cap 220C may comprise one or more reagents (e.g., lysis reagents, nucleic acid amplification reagents, CRISPR/Cas detection reagents). In some instances, for example, second cap 220C comprises one or more blister packs comprising one or more reagents. In some embodiments, reaction tube 220 comprises fluidic contents. In certain cases, the fluidic contents of reaction tube 220 comprise a reaction buffer. In certain embodiments, the reaction buffer comprises one or more buffers (e.g., phosphate-buffered saline (PBS), Tris). In certain embodiments, the reaction buffer comprises one or more salts. Reaction tube 220 may contain any suitable volume of the reaction buffer.

In operation, a user may collect a sample using sample-collecting component 210. In some instances, for example, the user may insert swab element 210A into a nasal or oral cavity of a subject (e.g., the user, a friend or family member of the user, or any other human or animal subject). Cap 220B may be removed from tube 220A (e.g., either before or after collection of the sample), thereby exposing the fluidic contents of tube 220A, and, after collecting the sample, swab element 210A may be inserted into the fluidic contents of tube 220A. In some cases, the user may stir swab element 210A in the fluidic contents of tube 220A for a period of time (e.g., at least 10 seconds, at least 20 seconds, at least 30 seconds). In certain instances, swab element 210A is removed from tube 220A. In certain other instances, stem element 210B is broken and removed such that swab element 210A remains in tube 220A.

After swab element 210A and/or stem element 210B is removed from tube 220A, a cap may be placed on tube 220A. In some instances, for example, second cap 220C may be placed on tube 220A. In some cases, tube 220A and/or second cap 220C comprise one or more reagents (e.g., lysis reagents, nucleic acid amplification reagents, CRISPR/Cas detection reagents). In certain embodiments, second cap 220C comprises one or more reagents. In some instances, the one or more reagents are in solid form (e.g., lyophilized, dried, crystallized, air jetted). In some cases, for example, the one or more reagents are in the form of one or more tablets and/or pellets. In certain instances, the one or more tablets and/or pellets comprise one or more coatings (e.g., a coating of a time release material). In some instances, the one or more reagents are in liquid form.

The one or more reagents may be released into reaction tube 220A by any suitable mechanism. In some cases, the one or more reagents may be released into tube 220A by inverting (and, in some cases, repeatedly inverting) reaction tube 220. In some cases, second cap 220C comprises a seal (e.g. a foil seal) separating the one or more reagents from the contents of tube 220A, and the seal may be punctured by screwing second cap 220C onto tube 220A, by puncturing the seal with a puncturing tool, or otherwise puncturing the seal. In some cases, the user presses on a button or other portion of second cap 220C and/or twists at least a portion of second cap 220C to release the one or more reagents into tube 220A.

In some embodiments, reaction tube 220 may be inserted into heater 240. Reaction tube 220 may be heated at one or more temperatures (e.g., at least 37° C., at least 65° C.) for one or more periods of time. In some cases, heating reaction tube 220 according to a first heating protocol (e.g., a first set of temperature(s) and time period(s)) may facilitate lysis of cells within the collected sample. In a particular, non-limiting embodiment, a first heating protocol comprises heating reaction tube 220 at 37° C. for 5-10 minutes (e.g., about 3 minutes) and at 65° C. for 5-10 minutes (e.g., about 10 minutes). In some cases, heating reaction tube 220 according to a second heating protocol (e.g., a second set of temperature(s) and time period(s)) may facilitate amplification of one or more target nucleic acids (if present within the sample). In a particular, non-limiting embodiment, a second heating protocol comprises heating reaction tube 220 at 37° C. for 10-15 minutes. In some cases, the heater may comprise an indicator (e.g., a visual indicator) that a heating protocol is occurring. The indicator may indicate to a user when the reaction tube should be removed from the device.

Following heating, reaction tube 220 may be inserted into “chimney” detection component 230. Upon insertion, reaction tube 220 may be punctured by a puncturing component (e.g., a blade, a needle) of “chimney” detection component 230. In some cases, at least a portion of the fluidic contents of reaction tube 220 are deposited onto a portion of a lateral flow assay strip of “chimney” detection component 230. The fluidic contents of reaction tube 220 may flow through the lateral flow assay strip (e.g., via capillary action), and the presence or absence of one or more target nucleic acid sequences may be indicated on a portion of the lateral flow assay strip (e.g., by the formation of one or more lines on the lateral flow assay strip). In some instances, for example, the portion of the lateral flow assay strip may be visible to a user (e.g., through an opening, a clear window, etc.). In some cases, software (e.g., a mobile application) may be used to read, analyze, and/or report the results (e.g., the one or more lines of the lateral flow assay strip). In some embodiments, “chimney” detection component 230 comprises one or more markings (e.g., ArUco markers) to facilitate to facilitate alignment of an electronic device (e.g., a smartphone, a tablet) with “chimney” detection component 230.

Although FIG. 10A shows an embodiment comprising a reaction tube comprising first cap 220B and second cap 220C, other embodiments may comprise a reaction tube comprising a single cap. In some such embodiments, the single cap may be a removable cap (e.g., a screw-top cap), a permanently-attached cap (e.g., a hinged cap), or any other type of cap. In some cases, the single cap may comprise one or more reagents (e.g., lysis reagents, nucleic acid amplification reagents, CRISPR/Cas detection reagents). In some cases, the one or more reagents may be in solid form (e.g., lyophilized, dried, crystallized, air jetted). In some cases, the one or more reagents may be in liquid form. In certain embodiments, a user may perform one or more actions (e.g., inverting tube 220A, screwing the cap onto tube 220A, pressing on a button or other portion of the cap, twisting at least a portion of the cap) to release the one or more reagents into tube 220A. In some cases, the single cap may not comprise any reagents, and any necessary reagents may be present in tube 220A.

In some embodiments, a diagnostic system comprises a reaction tube comprising at least two caps that each comprise one or more reagents (e.g., lysis reagents, nucleic acid amplification reagents, CRISPR/Cas detection reagents). In certain embodiments, the one or more reagents are in solid form (e.g., lyophilized, dried, crystallized, air jetted). In some cases, the at least two caps may be used to sequentially add reagents to a reaction tube.

FIG. 10B shows an embodiment of diagnostic system 200 comprising reaction tube 220 comprising tube 220A, first cap 220B, second cap 220C, and third cap 220D. In certain cases, second cap 220C and third cap 220D each comprise one or more reagents. In some cases, second cap 220C may contain a first set of reagents (e.g., lysis reagents), and third cap 220D may comprise a second set of reagents (e.g., nucleic acid amplification reagents). In some cases, caps may have different colors to indicate that they contain different reagents. For example, in FIG. 10B, second cap 220C is red, while third cap 220D is blue. In some cases, the first set of reagents and/or the second set of reagents are in solid form (e.g., lyophilized, dried, crystallized, air jetted). In certain cases, for example, the one or more reagents are in the form of one or more tablets and/or pellets. In certain instances, the one or more tablets and/or pellets comprise one or more coatings (e.g., a coating of a time release material). In some cases, coatings of different materials and/or thicknesses may delay release of one or more reagents to an appropriate time in the reaction and may facilitate the sequential adding of different reagents. In some instances, the one or more reagents are in liquid form. In addition to reaction tube 220, diagnostic system 200 may comprise sample-collecting component 210, “chimney” detection component 230, and heater 240.

In operation, a user may collect a sample using sample-collecting component 210, as described above. Cap 220B may be removed from tube 220A (e.g., either before or after collection of the sample), thereby exposing the fluidic contents of tube 220A, and, after collecting the sample, swab element 210A may be inserted into the fluidic contents of tube 220A. In some cases, the user may stir swab element 210A in the fluidic contents of tube 220A for a period of time (e.g., at least 10 seconds, at least 20 seconds, at least 30 seconds). In certain instances, swab element 210A is removed from tube 220A. In certain other instances, stem element 210B is broken and removed such that swab element 210A remains in tube 220A.

After swab element 210A and/or stem element 210B is removed from tube 220A, a cap may be placed on tube 220A. In some instances, for example, second cap 220C may be placed on tube 220A. In some cases, second cap 220C comprises one or more reagents (e.g., lysis reagents). In some instances, the one or more reagents are in solid form (e.g., lyophilized, dried, crystallized, air jetted). In some cases, for example, the one or more reagents are in the form of one or more tablets and/or pellets. In certain instances, the one or more tablets and/or pellets comprise one or more coatings (e.g., a coating of a time release material). In some instances, the one or more reagents are in liquid form.

The one or more reagents may be released from second cap 220C into tube 220A by any suitable mechanism. In some cases, the one or more reagents may be released into tube 220A by inverting (and, in some cases, repeatedly inverting) reaction tube 220. In some cases, second cap 220C comprises a seal (e.g. a foil seal) separating the one or more reagents from the contents of tube 220A, and the seal may be punctured by screwing second cap 220C onto tube 220A, by puncturing the seal with a puncturing tool, or otherwise puncturing the seal. In some cases, the user presses on a button or other portion of second cap 220C and/or twists at least a portion of second cap 220C to release the one or more reagents into tube 220A.

In some cases, after the one or more reagents contained in second cap 220C have been added into tube 220A, reaction tube 220 may be heated in heater 240 according to a first heating protocol. In certain embodiments, for example, heating reaction tube 220 according to the first heating protocol may facilitate lysis of cells within the collected sample. In a particular, non-limiting embodiment, a first heating protocol comprises heating reaction tube 220 at 37° C. for 5-10 minutes (e.g., about 3 minutes) and at 65° C. for 5-10 minutes (e.g., about 10 minutes).

After completion of the first heating protocol, second cap 220C may be removed from tube 220A, and third cap 220D may be placed on tube 220A. In some embodiments, third cap 220D comprises one or more reagents (e.g., nucleic acid amplification reagents). In some instances, the one or more reagents are in solid form (e.g., lyophilized, dried, crystallized, air jetted). In some cases, for example, the one or more reagents are in the form of one or more tablets and/or pellets. In certain instances, the one or more tablets and/or pellets comprise one or more coatings (e.g., a coating of a time release material). In some instances, the one or more reagents are in liquid form.

The one or more reagents may be released from third cap 220D into tube 220A by any suitable mechanism. In some cases, the one or more reagents may be released into tube 220A by inverting (and, in some cases, repeatedly inverting) reaction tube 220. In some cases, third cap 220D comprises a seal (e.g. a foil seal) separating the one or more reagents from the contents of tube 220A, and the seal may be punctured by screwing third cap 220D onto tube 220A, by puncturing the seal with a puncturing tool, or otherwise puncturing the seal. In some cases, the user presses on a button or other portion of third cap 220D and/or twists at least a portion of third cap 220D to release the one or more reagents into tube 220A.

In some cases, after the one or more reagents contained in third cap 220D have been added into tube 220A, reaction tube 220 may be heated in heater 240 according to a second heating protocol. In certain embodiments, for example, heating reaction tube 220 according to the second heating protocol may facilitate amplification of one or more target nucleic acid sequences (if present in the sample). In a particular, non-limiting embodiment, a second heating protocol comprises heating reaction tube 220 at 32° C. for 1-10 minutes (e.g., about 3 minutes), at 65° C. for 10-40 minutes, and at 37° C. for 10-20 minutes (e.g., about 15 minutes).

According to some embodiments, a diagnostic device comprises a “chimney” detection component. In some embodiments, the “chimney” detection component comprises a chimney, a front panel, and a bottom panel comprising a lateral flow assay strip and a puncturing component. As noted above, the chimney and the front panel may be integrally formed or may be separately formed. The chimney, the front panel, and the back panel may be formed from any suitable material(s). In some cases, for example, the chimney, the front panel, and/or the back panel comprise one or more thermoplastic materials and/or metals. In some embodiments, the chimney, the front panel, and/or the back panel may be manufactured by injection molding, an additive manufacturing technique (e.g., 3D printing), and/or a subtractive manufacturing technique (e.g., laser cutting).

The chimney may have suitable dimensions to receive a reaction tube. In certain embodiments, the chimney has a height of 60 mm or less, 55 mm or less, 50 mm or less, 45 mm or less, 40 mm or less, 35 mm or less, 30 mm or less, 25 mm or less, 20 mm or less, 15 mm or less, or 10 mm or less. In some embodiments, the chimney has a height in a range from 10 mm to 20 mm, 10 mm to 30 mm, 10 mm to 40 mm, 10 mm to 50 mm, 10 mm to 60 mm, 20 mm to 30 mm, 20 mm to 40 mm, 20 mm to 50 mm, 20 mm to 60 mm, 30 mm to 40 mm, 30 mm to 50 mm, 30 mm to 60 mm, 40 mm to 50 mm, 40 mm to 60 mm, or 50 mm to 60 mm. In certain embodiments, the chimney has an inner diameter of 30 mm or less, 25 mm or less, 20 mm or less, 15 mm or less, 10 mm or less, or 5 mm or less. In some embodiments, the chimney has an inner diameter of 5 mm to 10 mm, 5 mm to 15 mm, 5 mm to 20 mm, 5 mm to 25 mm, 5 mm to 30 mm, 10 mm to 15 mm, 10 mm to 20 mm, 10 mm to 25 mm, 10 mm to 30 mm, 15 mm to 20 mm, 15 mm to 25 mm, 15 mm to 30 mm, or 20 mm to 30 mm.

### Target Nucleic Acid Sequences

The diagnostic devices, systems, and methods, in some embodiments, may be used to detect the presence or absence of any target nucleic acid sequence (e.g., from any pathogen of interest). Target nucleic acid sequences may be associated with a variety of diseases or disorders, as described below. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are configured to identify particular strains of a pathogen (e.g., a virus). In certain embodiments, a diagnostic device comprises a lateral flow assay strip comprising a first test line configured to detect a nucleic acid sequence of SARS-CoV-2 and a second test line configured to detect a nucleic acid sequence of a SARS-CoV-2 virus having a D614G mutation (i.e., a mutation of the 614th amino acid from aspartic acid (D) to glycine (G)) in its spike protein. In some embodiments, one or more target nucleic acid sequences are associated with a single-nucleotide polymorphism (SNP). In certain cases, diagnostic devices, systems, and methods described herein may be used for rapid genotyping to detect the presence or absence of a SNP, which may affect medical treatment.

In some embodiments, the diagnostic devices, systems, and methods are configured to diagnose two or more diseases or disorders. In certain cases, for example, a diagnostic device comprises a lateral flow assay strip comprising a first test line configured to detect a nucleic acid sequence of SARS-CoV-2 and a second test line configured to detect a nucleic acid sequence of an influenza virus (e.g., an influenza A virus or an influenza B virus). In some embodiments, a diagnostic device comprises a lateral flow assay strip comprising a first test line configured to detect a nucleic acid sequence of a virus and a second test line configured to detect a nucleic acid sequence of a bacterium. In some embodiments, a diagnostic device comprises a lateral flow assay strip comprising three or more test lines (e.g., test lines configured to detect SARS-CoV-2, SARS-CoV-2 D614G, an influenza type A virus, and/or an influenza type B virus). In some embodiments, a diagnostic device comprises a lateral flow assay strip comprising four or more test lines (e.g., test lines configured to detect SARS-CoV-2, SARS-CoV-2 D614G, an influenza type A virus, and/or an influenza type B virus).

In some embodiments, a diagnostic device, system, or method is configured to detect at least 1, at least 2, at least 3, at least 4, at least 5, at least 6, at least 7, at least 8, at least 9, or at least 10 target nucleic acid sequences. In some embodiments, the diagnostic device is configured to detect 1 to 2 target nucleic acid sequences, 1 to 5 target nucleic acid sequences, 1 to 8 target nucleic acid sequences, 1 to 10 target nucleic acid sequences, 2 to 5 target nucleic acid sequences, 2 to 8 target nucleic acid sequences, 2 to 10 target nucleic acid sequences, 5 to 8 target nucleic acid sequences, 5 to 10 target nucleic acid sequences, or 8 to 10 target nucleic acid sequences. Each target nucleic acid sequence may independently be a nucleic acid of a pathogen (e.g., a viral, bacterial, fungal, protozoan, or parasitic pathogen) and/or a cancer cell.

In some embodiments, the diagnostic devices, systems, and methods are configured to detect a target nucleic acid sequence of a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the viral pathogen is SARS-CoV-2 and/or SARS-CoV-2 D614G. In certain embodiments, the viral pathogen is an influenza virus. The influenza virus may be an influenza A virus (e.g., H1N1, H3N2) or an influenza B virus.

Other viral pathogens include, but are not limited to, adenovirus; Herpes simplex, type 1; Herpes simplex, type 2; encephalitis virus; papillomavirus (e.g., human papillomavirus); Varicella zoster virus; Epstein-Barr virus; human cytomegalovirus; human herpesvirus, type 8; BK virus; JC virus; smallpox; polio virus; hepatitis A virus; hepatitis B virus; hepatitis C virus; hepatitis D virus; hepatitis E virus; human immunodeficiency virus (HIV); human bocavirus; parvovirus B19; human astrovirus; Norwalk virus; coxsackievirus; rhinovirus; Severe acute respiratory syndrome (SARS) virus; yellow fever virus; dengue virus; West Nile virus; Guanarito virus; Junin virus; Lassa virus; Machupo virus; Sabia virus; Crimean-Congo hemorrhagic fever virus; Ebola virus; Marburg virus; measles virus; mumps virus; rubella virus; Hendra virus; Nipah virus; Rabies virus; rotavirus; orbivirus; Coltivirus; Hantavirus; Middle East Respiratory Coronavirus; Zika virus; norovirus; Chikungunya virus; and Banna virus.

In some embodiments, the diagnostic devices, systems, and methods are configured to detect a target nucleic acid sequence of a bacterium (e.g., a bacterial pathogen). The bacterium may be a Gram-positive bacterium or a Gram-negative bacterium. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:17, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

In some embodiments, the diagnostic devices, systems, and methods are configured to detect a target nucleic acid sequence of a fungus (e.g., a fungal pathogen). Examples of fungal pathogens include, but are not limited to, Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

In some embodiments, the diagnostic devices, systems, and methods are configured to detect a target nucleic acid sequence of one or more protozoa (e.g., a protozoan pathogen). Examples of protozoan pathogens include, but are not limited to, Entamoeba histolytica, Giardia lambila, Trichomonas vaginalis, Trypanosoma brucei, T. cruzi, Leishmania donovani, Balantidium coli, Toxoplasma gondii, Plasmodium spp., and Babesia microti.

In some embodiments, the diagnostic devices, systems, and methods are configured to detect a target nucleic acid sequence of a parasite (e.g., a parasitic pathogen). Examples of parasitic pathogens include, but are not limited to, Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, and Wuchereria bancrofti.

In some embodiments, the diagnostic devices, systems, and methods are configured to detect a target nucleic acid sequence of a cancer cell. Cancer cells have unique mutations found in tumor cells and absent in normal cells. For example, the diagnostic devices, systems, and methods may be configured to detect a target nucleic acid sequence encoding a cancer neoantigen, a tumor-associated antigen (TAA), and/or a tumor-specific antigen (TSA). Examples of TAAs include, but are not limited to, MelanA (MART-I), gplOO (Pmel 17), tyrosinase, TRP-I, TRP-2, MAGE-I, MAGE-3, BAGE, GAGE-I, GAGE-2, pl5(58), CEA, RAGE, NY-ESO (LAGE), SCP-I, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, pl85erbB2, pl80erbB-3, c-met, nm-23H1, PSA, TAG-72-4, CA 19-9, CA 72-4, CAM 17.1, NuMa, K-ras, β-Catenin, CDK4, Mum-1, pl6, TAGE, PSMA, PSCA, CT7, telomerase, 43-9F, 5T4, 791Tgp72, alpha-fetoprotein, 3-HCG, BCA225, BTAA, CA 125, CA 15-3 (CA 27.29\BCAA), CA 195, CA 242, CA-50, CAM43, CD68VKP1, CO-029, FGF-5, G250, Ga733 (EpCAM), HTgp-175, M344, MA-50, MG7-Ag, MOV18, NB/70K, NY-CO-I, RCAS1, SDCCAG16, TA-90 (Mac-2 binding protein\cyclophilin C-associated protein), TAAL6, TAG72, TLP, and TPS5. Neoantigens, in some embodiments, arise from tumor proteins (e.g., tumor-associated antigens and/or tumor-specific antigens). In some embodiments, the neoantigen comprises a polypeptide comprising an amino acid sequence that is identical to a sequence of amino acids within a tumor antigen or oncoprotein (e.g., Her2, E7, tyrosinase-related protein 2 (Trp2), Myc, Ras, or vascular endothelial growth factor (VEGF)). In some embodiments, the amino acid sequence comprises at least 10, at least 15, at least 20, at least 25, at least 30, at least 35, at is least 40, at least 45, at least 50, at least 75, at least 100, at least 150, at least 200, or at least 250 amino acids. In some embodiments, the amino acid sequence comprises 10-250, 50-250, 100-250, or 50-150 amino acids.

In some embodiments, the diagnostic devices, systems, and methods are configured to examine a subject's predisposition to certain types of cancer based on specific genetic mutations. As an example, mutations in BRCA1 and/or BRCA2 may indicate that a subject is at an increased risk of breast cancer, as compared to a subject who does not have mutations in the BRCA1 and/or BRCA2 genes. In some instances, the diagnostic devices, systems, and methods are configured to detect a target nucleic acid sequence comprising a mutation in BRCA1 and/or BRCA2. Other genetic mutations that may be screened according to the diagnostic devices, systems, and methods provided herein include, but are not limited to, BARD1, BRIP1, TP53, PTEN, MSH2, MLH1, MSH6, NF1, PMS1, PMS2, EPCAM, APC, RB1, MEN1, MEN2, and VHL. Further, determining a subject's genetic profile may help guide treatment decisions, as certain cancer drugs are indicated for subjects having specific genetic variants of particular cancers. For example, azathioprine, 6-mercaptopurine, and thioguanine all have dosing guidelines based on a subject's thiopurine methyltransferase (TPMT) genotype (see, e.g., The Pharmacogeneomics Knowledgebase, pharmgkb.org).

In some embodiments, the diagnostic devices, systems, and methods are configured to detect a target nucleic acid sequence associated with a genetic disorder. Non-limiting examples of genetic disorders include hemophilia, sickle cell anemia, α-thalassemia, 0-thalassemia, Duchene muscular dystrophy (DMD), Huntington's disease, severe combined immunodeficiency, Marfan syndrome, hemochromatosis, and cystic fibrosis. In some embodiments, the target nucleic acid sequence is a portion of nucleic acid from a genomic locus of at least one of the following genes: CFTR, FMR1, SMN1, ABCB 11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASP A, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CUT A, CLN3, CLN5, CLN6, CLN8, CLRN1, CNGB3, COL27A1, COL4A3, COL4A4, COL4A5, COL7A1, CPS1, CPT1A, CPT2, CRB 1, CTNS, CTSK, CYBA, CYBB, CYP11B1, CYP11B2, CYP17A1, CYP19A1, CYP27A1, DBT, DCLRElC, DHCR7, DHDDS, DLD, DMD, DNAH5, DNAI1, DNAI2, DYSF, EDA, EIF2B5, EMD, ERCC6, ERCC8, ESC02, ETFA, ETFDH, ETHEl, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAM A3, LAMB3, LAMC2, LCA5, LDLR, LDLRAP1, LHX3, LIFR, LIP A, LOXHD1, LPL, LRPPRC, MAN2B1, MCOLN1, MED 17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OP A3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

In some embodiments, the diagnostic devices, systems, and methods are configured to detect a target nucleic acid sequence of an animal pathogen. Examples of animal pathogens include, but are not limited to, bovine rhinotracheitis virus, bovine herpesvirus, distemper, parainfluenza, canine adenovirus, rhinotracheitis virus, calicivirus, canine parvovirus, Borrelia burgdorferi (Lyme disease), Bordetella bronchiseptica (kennel cough), canine parainfluenza, leptospirosis, feline immunodeficiency virus, feline leukemia virus, Dirofilaria immitis (heartworm), feline herpesvirus, Chlamydia infections, Bordetella infections, equine influenza, rhinopneumonitis (equine herpesevirus), equine encephalomyelitis, West Nile virus (equine), Streptococcus equi, tetanus (Clostridium tetani), equine protozoal myeloencephalitis, bovine respiratory disease complex, clostridial disease, bovine respiratory syncytial virus, bovine viral diarrhea, Haemophilus somnus, Pasteurella haemolytica, and Pastuerella multocida.

The diagnostic devices, systems, and methods described herein may also be used to test water or food for contaminants (e.g., for the presence of one or more bacterial toxins). Bacterial contamination of food and water can result in foodborne diseases, which contribute to approximately 128,000 hospitalizations and 3000 deaths annually in the United States (CDC, 2016). In some cases, the diagnostic devices, systems, and methods described herein may be used to detect one or more toxins (e.g., bacterial toxins). In particular, bacterial toxins produced by Staphylococcus spp., Bacillus spp., and Clostridium spp. account for the majority of foodborne illnesses. Non-limiting examples of bacterial toxins include toxins produced by Clostridium botulinum, C. perfringens, Staphylococcus aureus, Bacillus cereus, Shiga-toxin-producing Escherichia coli (STEC), and Vibrio parahemolyticus. Exemplary toxins include, but are not limited to, aflatoxin, cholera toxin, diphtheria toxin, Salmonella toxin, Shiga toxin, Clostridium botulinum toxin, endotoxin, and mycotoxin. By testing a potentially contaminated food or water sample using the diagnostic devices, systems, or methods described herein, one can determine whether the sample contains the one or more bacterial toxins. In some embodiments, the diagnostic devices, systems, or methods may be operated or conducted during the food production process to ensure food safety prior to consumption.

In some embodiments, the diagnostic devices, systems, and methods described herein may be used to test samples of soil, building materials (e.g., drywall, ceiling tiles, wall board, fabrics, wall paper, and floor coverings), air filters, environmental swabs, or any other sample. In certain embodiments, the diagnostic devices, systems, and methods may be used to detect one or more toxins, as described above. In certain instances, the diagnostic devices, systems, and methods may be used to analyze ammonia- and methane-oxidizing bacteria, fungi or other biological elements of a soil sample. Such information can be useful, for example, in predicting agricultural yields and in guiding crop planting decisions.

### Diagnostic Systems

According to some embodiments, diagnostic systems comprise a sample-collecting component (e.g., a swab) and a diagnostic device. In certain cases, the diagnostic device comprises a cartridge (e.g., a microfluidic cartridge), a blister pack, and/or a “chimney” detection component. In some cases, the diagnostic device comprises a detection component (e.g., a lateral flow assay strip, a colorimetric assay). In certain embodiments, the diagnostic device further comprises one or more reagents (e.g., lysis reagents, nucleic acid amplification reagents, CRISPR/Cas detection reagents). In certain other embodiments, the diagnostic system separately includes one or more reaction tubes comprising the one or more reagents. Each of the one or more reagents may be in liquid form (e.g., in solution) or in solid form (e.g., lyophilized, dried, crystallized, air jetted). The diagnostic device may also comprise an integrated heater, or the diagnostic system may comprise a separate heater.

In some embodiments, the diagnostic system comprises a sample-collecting component. The sample-collecting component may be configured to collect a sample (e.g., a nasal secretion, an oral secretion, a cell scraping, blood, urine) from a subject (e.g., a human subject, an animal subject).

In some embodiments, the sample-collecting component comprises a swab element. In certain cases, the swab element comprises an absorbent material. Non-limiting examples of suitable absorbent materials include cotton, filter paper, cellulose, cellulose-derived materials, polyurethane, polyester, rayon, nylon, microfiber, viscose, and alginate. In some instances, the swab element is a foam swab and/or a flocked swab (e.g., comprising flocked fibers of a material). In some embodiments, the swab element comprises a thermoplastic polymer (e.g., a polystyrene, a polyolefin such as polyethylene or polypropylene) and/or a metal (e.g., aluminum). In some such embodiments, the swab element may be formed by injection molding, an additive manufacturing process (e.g., 3D printing), and/or a subtractive manufacturing process (e.g., laser cutting).

In certain embodiments, at least a portion of the swab element is wrapped in a material (e.g., plastic) to ensure sterility until use. In some embodiments, the swab element is pre-moistened. The swab element may have any suitable size and shape. In some embodiments, the swab element has a relatively small diameter (i.e., largest cross-sectional dimension). In certain cases, a relatively small diameter may facilitate insertion of the swab element into a nasal cavity (e.g., anterior nares) or an oral cavity of a subject. In certain cases, a relatively small diameter may facilitate insertion of the swab element (after sample collection) into a diagnostic system component (e.g., a reaction tube, a reservoir of a cartridge, a sample port of a blister pack). In certain embodiments, the swab element has a maximum diameter of 20 mm or less, 15 mm or less, 10 mm or less, 9 mm or less, 8 mm or less, 7 mm or less, 6 mm or less, 5 mm or less, 4 mm or less, 3 mm or less, 2 mm or less, or 1 mm or less. In some embodiments, the swab element has a maximum diameter in a range from 1 mm to 2 mm, 1 mm to 5 mm, 1 mm to 10 mm, 2 mm to 5 mm, 2 mm to 10 mm, 2 mm to 15 mm, 2 mm to 20 mm, 5 mm to 10 mm, 5 mm to 15 mm, 5 mm to 20 mm, 10 mm to 15 mm, or 10 mm to 20 mm.

In some embodiments, the swab element of the sample-collecting component is proximal to a stem element (e.g., a handle, an applicator). In certain cases, the stem element facilitates collection of a sample with the swab element. In some instances, for example, the stem element facilitates insertion of the swab element into a nasal cavity (e.g., anterior nares) or an oral cavity of a subject. The stem element may be formed from any suitable material. In some embodiments, the stem element comprises a thermoplastic polymer (e.g., a polystyrene, a polyolefin such as polyethylene or polypropylene), a metal (e.g., aluminum), wood, paper, and/or another type of material. In some embodiments, the stem element comprises one or more markings and/or flanges. The markings and/or flanges may, in some instances, facilitate sample collection by indicating the appropriate depth of insertion (e.g., into a nasal cavity).

In some embodiments, the sample-collecting component is a breakable swab comprising a swab element and a stem element. In some embodiments, the stem element comprises a breakable section. The breakable section may comprise any feature that facilitates separation of the stem portion and swab portion. In some cases, a breakable section comprises one or more cutouts, scored lines, and/or perforations. In certain embodiments, the breakable section of a stem element of a sample-collecting component has a narrower width than other sections of the stem element. The breakable section may facilitate separation of a stem element and a swab element of a sample-collecting component upon application of a relatively low amount of force (e.g., a manually applied force).

In certain embodiments, a user may use a swab element of a breakable swab to collect a sample. In some cases, the sample-containing swab element may be inserted into a diagnostic system component (e.g., a reaction tube, a reservoir of a cartridge, a sample port of a blister pack). A force may be applied to the breakable swab such that the stem element of the breakable swab is broken, and the stem element may be removed from the diagnostic system component. In some instances, the diagnostic system component may subsequently be covered (e.g., by a cap). In some embodiments, the sample-containing swab element may be further processed without contamination of the sample.

In some cases, a breakable swab may advantageously increase nucleic acid concentration in reaction mixture (e.g., by increasing incubation time of a sample-bearing swab element in a reaction buffer, thereby facilitating transfer of a sample collected on the swab element to the reaction buffer). In some cases, a breakable swab may advantageously reduce risk of contaminating a collected sample.

In some embodiments, a diagnostic system comprises a diagnostic device. As described below, the diagnostic device may comprise a cartridge (e.g., a microfluidic cartridge), a blister pack, and/or a “chimney” detection component.

In some embodiments, the diagnostic system comprises one or more reagents (e.g., lysis reagents, nucleic acid amplification reagents, CRISPR/Cas detection reagents). In some instances, at least one reagent is contained within a diagnostic device (e.g., a cartridge, a blister pack, a “chimney” detection component) of a diagnostic system. In some instances, at least one reagent is provided separately from the diagnostic device. In certain cases, for example, the diagnostic system comprises one or more reaction tubes comprising the at least one reagent.

In some embodiments, at least one (and, in some instances, each) of the one or more reagents is in liquid form (e.g., in solution). In some embodiments, at least one (and, in some instances, each) of the one or more reagents is in solid form. In certain embodiments, at least one (and, in some instances, each) of the one or more reagents is in solid form (e.g., lyophilized, dried, crystallized, air jetted).

In certain embodiments, the one or more reagents comprise one or more lysis reagents. A lysis reagent generally refers to a reagent that promotes cell lysis either alone or in combination with one or more reagents and/or conditions (e.g., heating). In some cases, the one or more lysis reagents comprise one or more enzymes. Non-limiting examples of suitable enzymes include lysozyme, lysostaphin, zymolase, cellulose, protease, and glycanase. In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40.

In some embodiments, the one or more lysis reagents comprise an RNase inhibitor (e.g., a murine RNase inhibitor). In certain embodiments, the RNase inhibitor concentration is at least 0.1 U/μL, at least 0.2 U/μL, at least 0.5 U/μL, at least 0.8 U/μL, at least 1.0 U/μL, at least 1.2 U/μL, at least 1.5 U/μL, at least 1.8 U/μL, or at least 2.0 U/μL. In certain embodiments, the RNase inhibitor concentration is in a range from 0.1 U/μL to 0.2 U/μL, 0.1 U/μL to 0.5 U/μL, 0.1 U/μL to 1.0 U/μL, 0.1 U/μL to 1.5 U/μL, 0.1 U/μL to 2.0 U/μL, 0.5 U/μL to 1.0 U/μL, 0.5 U/μL to 1.5 U/μL, 0.5 U/μL to 2.0 U/μL, or 1.0 U/μL to 2.0 U/μL. In some embodiments, the one or more lysis reagents comprise Tween (e.g., Tween 20, Tween 80).

In some embodiments, the one or more reagents comprise one or more reagents to reduce or eliminate potential carryover contamination from prior tests (e.g., prior tests conducted in the same area). In some embodiments, the one or more reagents comprise thermolabile uracil DNA glycosylase (UDG). In some cases, UDG may prevent carryover contamination from prior tests by degrading products that have already been amplified (i.e., amplicons) while leaving unamplified samples untouched and ready for amplification. In some embodiments, the concentration of UDG is at least 0.01 U/μL, at least 0.02 U/μL, at least 0.03 U/μL, at least 0.04 U/μL, or at least 0.05 U/μL. In certain embodiments, the concentration of UDG is in a range from 0.01 U/μL to 0.02 U/μL, 0.01 U/μL to 0.03 U/μL, 0.01 U/μL to 0.04 U/μL, or 0.01 U/μL to 0.05 U/μL.

In certain embodiments, the one or more reagents comprise one or more reverse transcription reagents. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification. In some embodiments, the one or more reverse transcription reagents facilitate such reverse transcription. In certain instances, the one or more reverse transcription reagents comprise a reverse transcriptase, a DNA-dependent polymerase, and/or a ribonuclease (RNase). A reverse transcriptase generally refers to an enzyme that transcribes RNA to complementary DNA (cDNA) by polymerizing deoxyribonucleotide triphosphates (dNTPs). An RNase generally refers to an enzyme that catalyzes the degradation of RNA. In some cases, an RNase may be used to digest RNA from an RNA-DNA hybrid.

In some embodiments, the one or more reagents comprise one or more nucleic acid amplification reagents. A nucleic acid amplification reagent generally refers to a reagent that facilitates a nucleic acid amplification method. In some embodiments, the nucleic acid amplification method is an isothermal nucleic acid amplification method. In some cases, an isothermal nucleic acid amplification method, unlike PCR, avoids use of expensive, bulky laboratory equipment for precise thermal cycling. Non-limiting examples of suitable isothermal nucleic acid amplification methods include loop-mediated isothermal amplification (LAMP), recombinase polymerase amplification (RPA), nicking enzyme amplification reaction (“NEAR”), thermophilic helicase dependent amplification (tHDA), nucleic acid sequence-based amplification (NASBA), strand displacement amplification (SDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In certain embodiments, the one or more nucleic acid amplification reagents comprise LAMP reagents, RPA reagents, or NEAR reagents.

In some embodiments, the one or more reagents comprise one or more additives that enhance reagent stability (e.g., protein stability). Non-limiting examples of suitable additives include trehalose, polyethylene glycol (PEG), polyvinyl alcohol (PVA), and glycerol.

In some embodiments, the one or more reagents comprise one or more buffers. Non-limiting examples of suitable buffers include phosphate-buffered saline (PBS) and Tris. In some embodiments, the one or more buffers have a relatively neutral pH. In some embodiments, the one or more buffers have a pH in a range from 5.0 to 6.0, 5.0 to 7.0, 5.0 to 8.0, 5.0 to 9.0, 6.0 to 7.0, 6.0 to 8.0, 6.0 to 9.0, 7.0 to 8.0, 7.0 to 9.0, or 8.0 to 9.0. In some embodiments, the one or more reagents comprise one or more salts. Non-limiting examples of suitable salts include magnesium acetate tetrahydrate, potassium acetate, and potassium chloride.

In some embodiments, at least one reagent is not contained within a diagnostic device, and a diagnostic system comprises one or more reaction tubes. The one or more reaction tubes may contain any reagent(s) described above. In some embodiments, the one or more reaction tubes comprise at least one reagent in liquid form. In some embodiments, the one or more reaction tubes comprise at least one reagent in solid form.

A reaction tube of a diagnostic system may be formed from any suitable material. In some embodiments, the reaction tube is formed from a polymer. Non-limiting examples of suitable polymers include polypropylene (PP), polytetrafluoroethylene (PTFE), polyurethane (PU), polyvinyl chloride (PVC), polystyrene, neoprene, nitrile, nylon and polyamide. In some embodiments, the reaction tube comprises glass and/or a ceramic. The glass may, in some instances, be an expansion-resistant glass (e.g., borosilicate glass or fused quartz). In some embodiments, the reaction tube is an Eppendorf tube. In some embodiments, the reaction tube has a substantially flat bottom (e.g., the reaction tube can stand on its own), a substantially round bottom, or a substantially conical bottom. If the reaction tube has a round or conical bottom, or any other bottom that does not allow the reaction tube to readily stand on its own, the diagnostic system may further comprise a stand for the reaction tube. In some embodiments, the reaction tube is sterile.

The reaction tubes, in some embodiments, further comprise at least one cap. In some embodiments, the reaction tube comprises a partially removable cap (e.g., a hinged cap) or one or more wholly removable caps (e.g., one or more screw-top caps, one or more stoppers). In some embodiments, the one or more caps comprise reagents in solid form (e.g., lyophilized, dried, crystallized, air jetted reagents).

The reaction tube may be configured to hold any suitable volume of liquid. In some embodiments, the reaction tube is configured to hold a volume of at least 5 μL, at least 10 μL, at least 15 μL, at least 20 μL, at least 25 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 400 μL, at least 500 μL, at least 600 μL, at least 700 μL, at least 800 μL, at least 900 μL, at least 1 mL, at least 1.5 mL, or at least 2 mL. In some embodiments, the reaction tube is configured to hold a volume in a range from 5 μL to 10 μL, 5 μL to 20 μL, 5 μL to 50 μL, 5 μL to 70 μL, 5 μL to 100 μL, 5 μL to 200 μL, 5 μL to 500 μL, 5 μL to 1 mL, 5 μL to 1.5 mL, 5 μL to 2 mL, 10 μL to 20 μL, 10 μL to 50 μL, 10 μL to 70 μL, 10 μL to 100 μL, 10 μL to 200 μL, 10 μL to 500 μL, 10 μL to 1 mL, 10 μL to 1.5 mL, 10 μL to 2 mL, 20 μL to 50 μL, 20 μL to 70 μL, 20 μL to 100 μL, 20 μL to 200 μL, 20 μL to 500 μL, 20 μL to 1 mL, 20 μL to 1.5 mL, 20 μL to 2 mL, 50 μL to 70 μL, 50 μL to 100 μL, 50 μL to 200 μL, 50 μL to 500 μL, 50 μL to 1 mL, 50 μL to 1.5 mL, 50 μL to 2 mL, 70 μL to 100 μL, 70 μL to 200 μL, 70 μL to 500 μL, 70 μL to 1 mL, 70 μL to 1.5 mL, 70 μL to 2 mL, 100 μL to 200 μL, 100 μL to 500 μL, 100 μL to 1 mL, 100 μL to 1.5 mL, 100 μL to 2 mL, 200 μL to 500 μL, 200 μL to 1 mL, 200 μL to 1.5 mL, 200 μL to 2 mL, 500 μL to 1 mL, 500 μL to 1.5 mL, 500 μL to 2 mL, 1 mL to 1.5 mL, or 1 mL to 2 mL.

In some embodiments, the reaction tube contains a volume of liquid (i.e., fluidic contents). In certain embodiments, the fluidic contents of the reaction tube have a volume sufficient to facilitate fluid flow through a lateral flow assay strip. In some embodiments, the fluidic contents of the reaction tube have an initial volume of at least 5 μL, at least 10 μL, at least 15 μL, at least 20 μL, at least 25 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 400 μL, at least 500 μL, at least 600 μL, at least 700 μL, at least 800 μL, at least 900 μL, at least 1 mL, at least 1.5 mL, or at least 2 mL. In some embodiments, the fluidic contents of the reaction tube have an initial volume in a range from 5 μL to 10 μL, 5 μL to 20 μL, 5 μL to 50 μL, 5 μL to 70 μL, 5 μL to 100 μL, 5 μL to 200 μL, 5 μL to 500 μL, 5 μL to 1 mL, 5 μL to 1.5 mL, 5 μL to 2 mL, 10 μL to 20 μL, 10 μL to 50 μL, 10 μL to 70 μL, 10 μL to 100 μL, 10 μL to 200 μL, 10 μL to 500 μL, 10 μL to 1 mL, 10 μL to 1.5 mL, 10 μL to 2 mL, 20 μL to 50 μL, 20 μL to 70 μL, 20 μL to 100 μL, 20 μL to 200 μL, 20 μL to 500 μL, 20 μL to 1 mL, 20 μL to 1.5 mL, 20 μL to 2 mL, 50 μL to 70 μL, 50 μL to 100 μL, 50 μL to 200 μL, 50 μL to 500 μL, 50 μL to 1 mL, 50 μL to 1.5 mL, 50 μL to 2 mL, 70 μL to 100 μL, 70 μL to 200 μL, 70 μL to 500 μL, 70 μL to 1 mL, 70 μL to 1.5 mL, 70 μL to 2 mL, 100 μL to 200 μL, 100 μL to 500 μL, 100 μL to 1 mL, 100 μL to 1.5 mL, 100 μL to 2 mL, 200 μL to 500 μL, 200 μL to 1 mL, 200 μL to 1.5 mL, 200 μL to 2 mL, 500 μL to 1 mL, 500 μL to 1.5 mL, 500 μL to 2 mL, 1 mL to 1.5 mL, or 1 mL to 2 mL.

In some embodiments, the fluidic contents of the reaction tube comprise a reaction buffer. In certain instances, the reaction buffer comprises one or more buffers. Non-limiting examples of suitable buffers include phosphate-buffered saline (“PBS”) and Tris. In certain instances, the reaction buffer comprises one or more salts. Non-limiting examples of suitable salts include magnesium acetate tetrahydrate, potassium acetate, and potassium chloride.

In some embodiments, the reaction buffer comprises Tween (e.g., Tween 20, Tween 80). In some embodiments, the reaction buffer comprises an RNase inhibitor. In certain instances, Tween and/or an RNase inhibitor may facilitate cell lysis.

In a particular, non-limiting embodiment, the reaction buffer comprises 25 mM Tris buffer, 5% (w/v) poly(ethylene glycol) 35,000 kDa, 14 mM magnesium acetate tetrahydrate, 100 mM potassium acetate, and greater than 85% volume nuclease free water.

In some embodiments, the reaction buffer has a relatively neutral pH. In some embodiments, the reaction buffer has a pH in a range from 5.0 to 6.0, 5.0 to 7.0, 5.0 to 8.0, 5.0 to 9.0, 6.0 to 7.0, 6.0 to 8.0, 6.0 to 9.0, 7.0 to 8.0, 7.0 to 9.0, or 8.0 to 9.0.

In certain embodiments, the diagnostic device (e.g., cartridge, blister pack, “chimney” detection component) comprises a detection component. In some embodiments, the detection component comprises a lateral flow assay strip or a colorimetric assay. Examples of both are provided herein. In some embodiments, results of the lateral flow assay strip and/or colorimetric assay are read and/or analyzed by software (e.g., a mobile application).

In some embodiments, the diagnostic device comprises a lateral flow assay strip. In some embodiments, the lateral flow assay strip is configured to detect one or more target nucleic acid sequences. In certain cases, the lateral flow assay strip comprises one or more fluid-transporting layers comprising one or more materials that allow fluid transport (e.g., via capillary action). Non-limiting examples of suitable materials include polyethersulfone, cellulose, polycarbonate, nitrocellulose, sintered polyethylene, and glass fibers.

In some embodiments, the one or more fluid-transporting layers of the lateral flow assay strip comprise a plurality of fibers (e.g., woven or non-woven fabrics). In some embodiments, the one or more fluid-transporting layers comprise a plurality of pores. In some embodiments, pores and/or interstices between fibers may advantageously facilitate fluid transport (e.g., via capillary action). The pores may have any suitable average pore size. In certain embodiments, the plurality of pores has an average pore size of 30 μm or less, 25 μm or less, 20 μm or less, 15 μm or less, 10 μm or less, 5 μm or less, 2 μm or less, 1 μm or less, 0.9 μm or less, 0.8 μm or less, 0.7 μm or less, 0.6 μm or less, 0.5 μm or less, 0.4 μm or less, 0.3 μm or less, 0.2 μm or less, or 0.1 μm or less. In certain embodiments, the plurality of pores has an average pore size of at least 0.1 μm, at least 0.2 μm, at least 0.3 μm, at least 0.4 μm, at least 0.5 μm, at least 0.6 μm, at least 0.7 μm, at least 0.8 μm, at least 0.9 μm, at least 1 μm, at least 2 μm, at least 5 μm, at least 10 μm, at least 15 μm, at least 20 μm, at least 25 μm, or at least 30 μm. In some embodiments, the plurality of pores has an average pore size in a range from 0.1 μm to 0.5 p m, 0.1 μm to 1 p m, 0.1 μm to 5 μm, 0.1 μm to 10 μm, 0.1 μm to 15 μm, 0.1 μm to 20 μm, 0.1 μm to 25 μm, 0.1 μm to 30 μm, 0.5 μm to 1 μm, 0.5 μm to 5 μm, 0.5 μm to 10 μm, 0.5 μm to 15 μm, 0.5 μm to 20 μm, 0.5 μm to 25 μm, 0.5 μm to 30 μm, 1 μm to 5 μm, 1 μm to 10 μm, 1 μm to 15 μm, 1 μm to 20 μm, 1 μm to 25 μm, 1 μm to 30 μm, 5 μm to 10 μm, 5 μm to 15 μm, 5 μm to 20 μm, 5 μm to 25 μm, 5 μm to 30 μm, 10 μm to 15 μm, 10 μm to 20 μm, 10 μm to 25 μm, 10 μm to 30 μm, 15 μm to 20 μm, 15 μm to 25 μm, 15 μm to 30 μm, or 20 μm to 30 μm.

The one or more fluid-transporting layers of the lateral flow assay strip may have any suitable porosity. In some embodiments, the one or more fluid-transporting layers have a porosity of at least 10%, at least 20%, at least 30%, at least 40%, at least 50%, or at least 60%. In some embodiments, the one or more fluid-transporting layers have a porosity in a range from 10% to 20%, 10% to 30%, 10% to 40%, 10% to 50%, 10% to 60%, 20% to 40%, 20% to 50%, 20% to 60%, 30% to 50%, 30% to 60%, 40% to 60%, or 50% to 60%.

In certain embodiments, the lateral flow assay strip comprises one or more sub-regions. In some instances, the lateral flow assay strip comprises a first sub-region (e.g., a sample pad) where a fluidic sample is introduced to the lateral flow assay strip. In some instances, the lateral flow assay strip comprises a second sub-region (e.g., a particle conjugate pad) comprising a plurality of labeled particles. In some cases, the particles comprise gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG).

In certain embodiments, the lateral flow assay strip comprises a third sub-region (e.g., a test pad) comprising one or more test lines. In some embodiments, a first test line comprises a capture reagent (e.g., an immobilized antibody) configured to detect a first target nucleic acid sequence. In certain embodiments, the lateral flow assay strip comprises one or more additional test lines. In some instances, each test line of the lateral flow assay strip is configured to detect a different target nucleic acid. In some instances, two or more test lines of the lateral flow assay strip are configured to detect the same target nucleic sequence. The test line(s) may have any suitable shape or pattern (e.g., one or more straight lines, curved lines, dots, squares, check marks, x marks).

In certain embodiments, the third sub-region (e.g., the test pad) of the lateral flow assay strip comprises one or more control lines. In certain instances, a first control line is a human (or animal) nucleic acid control line. In some embodiments, for example, the human (or animal) nucleic acid control line is configured to detect a nucleic acid (e.g., RNase P) that is generally present in all humans (or animals). In some cases, the human (or animal) nucleic acid control line becoming detectable indicates that a human (or animal) sample was successfully collected, nucleic acids from the sample were amplified, and the amplicons were transported through the lateral flow assay strip. In certain instances, a first control line is a lateral flow control line. In some cases, the lateral flow control line becoming detectable indicates that a liquid was successfully transported through the lateral flow assay strip. In some embodiments, the lateral flow assay strip comprises two or more control lines. The control line(s) may have any suitable shape or pattern (e.g., one or more straight lines, curved lines, dots, squares, check marks, x marks). In some instances, for example, the lateral flow assay strip comprises a human (or animal) nucleic acid control line and a lateral flow control line.

In certain embodiments, the lateral flow assay strip comprises a fourth sub-region (e.g., a wicking area) to absorb fluid flowing through the lateral flow assay strip.

In some embodiments, the diagnostic device comprises a plurality of lateral flow assay strips. In certain cases, the plurality of lateral flow assay strips may be connected such that a fluidic sample may flow from a first end to a second end of a first lateral flow assay strip (e.g., via capillary action) and may then flow from the second end of the first lateral flow assay trip to a first end of a second lateral flow assay strip. In certain instances, the diagnostic device comprises a series of lateral flow strips that snap or lock together. In some cases, the diagnostic device comprises one or more lateral flow assay strips that have been impregnated with one or more reagents (e.g., lysis reagents, nucleic acid amplification reagents, CRISPR/Cas detection reagents). In certain embodiments, the one or more reagents may be in solid form (e.g., lyophilized, dried, crystallized, air jetted), and one or more buffers may be added to activate the solid reagents and move the sample to the next strip. In some embodiments, the strips have dams or gaps to impede fluid flow to give a reaction (e.g., lysis, amplification) sufficient time to occur.

In some embodiments, the diagnostic device comprises a colorimetric assay. In certain embodiments, the colorimetric assay comprises a cartridge comprising a central sample chamber in fluidic communication with a plurality of peripheral chambers (e.g., at least four peripheral chambers). In some embodiments, each peripheral chamber comprises isothermal nucleic acid amplification reagents comprising a unique set of primers (e.g., primers specific for one or more target nucleic acid sequences, primers specific for a positive test control, primers specific for a negative test control).

In operation, a sample may be deposited in the central sample chamber. In some cases, the sample may be combined with a reaction buffer in the central sample chamber. In certain cases, the central sample chamber may be heated to lyse cells within the sample. In some cases, the lysate may be directed to flow from the central sample chamber to the plurality of peripheral chambers comprising unique primers. In some cases, a colorimetric reaction may occur in each peripheral chamber, resulting in varying colors in the peripheral chambers. In some cases, the results within each peripheral chamber may be visible (e.g., through a clear film or other covering).

The diagnostic system, in some embodiments, comprises a heater. In certain embodiments, the heater is integrated with the diagnostic device. In some instances, for example, the heater is a printed circuit board (PCB) heater. The PCB heater, in some embodiments, comprises a bonded PCB with a microcontroller, thermistors, and/or resistive heaters. In certain embodiments, the diagnostic device comprises a cartridge and/or a blister pack comprising one or more reservoirs (e.g., a lysis reservoir, a nucleic acid amplification reservoir). In some embodiments, the PCB heater is in thermal communication with at least one of the one or more reservoirs. In some embodiments, the PCB heater is located adjacent to (e.g., below) at least one of the one or more reservoirs. amplification reservoirs)

In some embodiments, the diagnostic system comprises a separate heater (i.e., a heater that is not integrated with other system components). In some cases, the heater comprises a battery-powered heat source, a USB-powered heat source, a hot plate, a heating coil, and/or a hot water bath. In certain embodiments, the heating unit is contained within a thermally-insulated housing to ensure user safety. In certain instances, the heating unit is an off-the-shelf consumer-grade device. In some embodiments, the heat source is a thermocycler or other specialized laboratory equipment known in the art. In some embodiments, the heater is configured to receive a reaction tube.

In some embodiments, the heater is configured to heat one or more components of a diagnostic system (e.g., fluidic contents of a reaction tube or a reservoir) at a temperature of at least 37° C., at least 40° C., at least 45° C., at least 50° C., at least 55° C., at least 60° C., at least 65° C., at least 70° C., at least 75° C., at least 80° C., at least 85° C., or at least 90° C. In some embodiments, the heater is configured to heat one or more components of a diagnostic system (e.g., fluidic contents of a reaction tube or a reservoir) at a temperature in a range from 37° C. to 60° C., 37° C. to 70° C., 37° C. to 80° C., 37° C. to 90° C., 40° C. to 60° C., 40° C. to 70° C., 40° C. to 80° C., 40° C. to 90° C., 50° C. to 60° C., 50° C. to 70° C., 50° C. to 80° C., 50° C. to 90° C., 60° C. to 70° C., 60° C. to 80° C., 60° C. to 90° C., 70° C. to 80° C., 70° C. to 90° C., or 80° C. to 90° C.

In some embodiments, the heater is configured to heat one or more components of a diagnostic system (e.g., fluidic contents of a reaction tube or a reservoir) at a temperature for at least 5 minutes, at least 10 minutes, at least 15 minutes, at least 30 minutes, at least 45 minutes, at least 60 minutes, or at least 90 minutes. In certain embodiments, the heating unit is configured to heat one or more components of a diagnostic system (e.g., fluidic contents of a reaction tube or a reservoir) at a desired temperature for a time in a range from 5 minutes to 10 minutes, 5 minutes to 15 minutes, 5 minutes to 30 minutes, 5 minutes to 45 minutes, 5 minutes to 60 minutes, 5 minutes to 90 minutes, 10 minutes to 15 minutes, 10 minutes to 30 minutes, 10 minutes to 45 minutes, 10 minutes to 60 minutes, 10 minutes to 90 minutes, 15 minutes to 30 minutes, 15 minutes to 45 minutes, 15 minutes to 60 minutes, 15 minutes to 90 minutes, 30 minutes to 45 minutes, 30 minutes to 60 minutes, 30 minutes to 90 minutes, or 60 minutes to 90 minutes.

In some embodiments, the heater comprises at least two temperature zones. In certain instances, for example, the heater is an off-the-shelf consumer-grade heating coil connected to a microcontroller that is used to switch between two temperature zones. In some embodiments, the first temperature zone is in a range from 60° C. to 100° C., 60° C. to 90° C., 60° C. to 80° C., 60° C. to 70° C., or 60° C. to 65° C. In certain cases, the first temperature zone has a temperature of approximately 65° C. In some embodiments, the second temperature zone is in a range from 30° C. to 40° C. In certain cases, the second temperature zone has a temperature of approximately 37° C.

In some embodiments, the heater is configured to heat one or more components of a diagnostic system (e.g., fluidic contents of a reaction tube or a reservoir) to a plurality of temperatures for a plurality of time periods. In some embodiments, for example, a heater is configured to heat one or more components of a diagnostic system (e.g., fluidic contents of a reaction tube or a reservoir) at a first temperature for a first period of time and at a second temperature for a second period of time. The first temperature and the second temperature may be the same or different, and the first period of time and the second period of time may be the same or different.

In some embodiments, the heater is pre-programmed with one or more protocols. In some embodiments, for example, the heater is pre-programmed with a lysis heating protocol and/or an amplification heating protocol. A lysis heating protocol generally refers to a set of one or more temperatures and one or more time periods that facilitate lysis of the sample. An amplification heating protocol generally refers to a set of one or more temperatures and one or more time periods that facilitate nucleic acid amplification. In some embodiments, the heater comprises an auto-start mechanism that corresponds to the temperature profile needed for lysis and/or amplification. That is, a user may insert a reaction tube into the heater, and the heater may automatically run a lysis and/or amplification heating protocol. In some embodiments, the heater is controlled by a mobile application.

In some embodiments, a diagnostic system comprises instructions associated with system components. The instructions may include instructions for performing any one of the diagnostic methods provided herein. The instructions may include instructions for the use, modification, mixing, diluting, preserving, administering, assembly, storage, packaging, and/or preparation of system components. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.), and/or provided via electronic communications (including Internet or web-based communications). In some embodiments, the instructions are provided as part of a software-based application, as described herein.

In some embodiments, one or more components of a diagnostic system comprise a unique label. In some cases, this may advantageously allow multiple samples to be run in parallel. For example, one or more components of the diagnostic system (e.g., reaction tube cap, detection component) may be labeled with the same label. In some embodiments, a copy of the label is given to a tested subject, so that the subject may later receive the results using the unique label. In this way, multiple tests (one for each unique subject) may be run in parallel without mixing up the samples.

### Sample Collection

In some embodiments, a diagnostic method comprises collecting a sample from a subject (e.g., a human subject, an animal subject). In some embodiments, a diagnostic system comprises a sample-collecting component configured to collect a sample from a subject (e.g., a human subject, an animal subject). Exemplary samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, the sample comprises a nasal secretion. In certain instances, for example, the sample is an anterior nares specimen. An anterior nares specimen may be collected from a subject by inserting a swab element of a sample-collecting component into one or both nostrils of the subject for a period of time. In some embodiments, the period of time is at least 5 seconds, at least 10 seconds, at least 20 seconds, or at least 30 seconds. In some embodiments, the period of time is 30 seconds or less, 20 seconds or less, 10 seconds or less, or 5 seconds or less. In some embodiments, the period of time is in a range from 5 seconds to 10 seconds, 5 seconds to 20 seconds, 5 seconds to 30 seconds, 10 seconds to 20 seconds, or 10 seconds to 30 seconds. In some embodiments, the sample comprises a cell scraping. In certain embodiments, the cell scraping is collected from the mouth or interior cheek. The cell scraping may be collected using a brush or scraping device formulated for this purpose. The sample may be self-collected by the subject or may be collected by another individual (e.g., a family member, a friend, a coworker, a health care professional) using a sample-collecting component described herein.

In some embodiments, the sample comprises an oral secretion (e.g., saliva). In certain cases, the volume of saliva in the sample is at least 1 mL, at least 1.5 mL, at least 2 mL, at least 2.5 mL, at least 3 mL, at least 3.5 mL, or at least 4 mL. In some embodiments, the volume of saliva in the sample is in a range from 1 mL to 2 mL, 1 mL to 3 mL, 1 mL to 4 mL, or 2 mL to 4 mL. Saliva has been found to have a mean concentration of SARS-Cov-2 RNA of 5 fM (Kai-Wang To et al., 2020)—an amount that is detectable by any one of the methods described herein.

In some embodiments, the saliva sample is deposited directly into a reaction tube. In some embodiments, the concentration of a target nucleic acid molecule (e.g., SARS-CoV-2 RNA) in the sample is at least 5 aM, at least 10 aM, at least 15 aM, at least 20 aM, at least 25 aM, at least 30 aM, at least 35 aM, at least 40 aM, at least 50 aM, at least 75 aM, at least 100 aM, at least 150 aM, at least 200 aM, at least 300 aM, at least 400 aM, at least 500 aM, at least 600 aM, at least 700 aM, at least 800 aM, at least 900 aM, at least 1 fM, at least 5 fM, at least 10 fM, at least 15 fM, at least 20 fM, at least 25 fM, at least 30 fM, at least 35 fM, at least 40 fM, at least 50 fM, at least 75 fM, at least 100 fM, at least 150 fM, at least 200 fM, at least 300 fM, at least 400 fM, at least 500 fM, at least 600 fM, at least 700 fM, at least 800 fM, at least 900 fM, at least 1 pM, at least 5 pM, or at least 10 pM. In some embodiments, the concentration of a target nucleic acid molecule (e.g., SARS-CoV-2 RNA) in the sample is 10 pM or less, 5 pM or less, 1 pM or less, 500 fM or less, 100 fM or less, 50 fM or less, 10 fM or less, 1 fM or less, 500 aM or less, 100 aM or less, 50 aM or less 10 aM or less, or 5 aM or less. In some embodiments, the concentration of a target nucleic acid molecule (e.g., SARS-CoV-2 RNA) in the sample is in a range from 5 aM to 50 aM, 5 aM to 100 aM, 5 aM to 500 aM, 5 aM to 1 fM, 5 aM to 10 fM, 5 aM to 50 fM, 5 aM to 100 fM, 5 aM to 500 fM, 5 aM to 1 pM, 5 aM to 10 pM, 10 aM to 50 aM, 10 aM to 100 aM, 10 aM to 500 aM, 10 aM to 1 fM, 10 aM to 10 fM, 10 aM to 50 fM, 10 aM to 100 fM, 10 aM to 500 fM, 10 aM to 1 pM, 10 aM to 10 pM, 100 aM to 500 aM, 100 aM to 1 fM, 100 aM to 10 fM, 100 aM to 50 fM, 100 aM to 100 fM, 100 aM to 500 fM, 100 aM to 1 pM, 100 aM to 10 pM, 1 fM to 10 fM, 1 fM to 50 fM, 1 fM to 100 fM, 1 fM to 500 fM, 1 fM to 1 pM, 1 fM to 10 pM, 5 fM to 10 fM, 5 fM to 50 fM, 5 fM to 100 fM, 5 fM to 500 fM, 5 fM to 1 pM, 5 fM to 10 pM, 10 fM to 100 fM, 10 fM to 500 fM, 10 fM to 1 pM, 10 fM to 10 pM, 100 fM to 500 fM, 100 fM to 1 pM, 100 fM to 10 pM, or 1 pM to 10 pM.

The sample, in some embodiments, is collected from a subject who is suspected of having the disease(s) the test screens for, such as a coronavirus (e.g., COVID-19) and/or influenza (e.g., influenza type A or influenza type B). Other indications, as described herein, are also envisioned. In some embodiments, the subject is a human. Subjects may be asymptomatic, or may present with one or more symptoms of the disease(s). Symptoms of coronaviruses (e.g., COVID-19) include, but are not limited to, fever, cough (e.g., dry cough), generalized fatigue, sore throat, headache, loss of taste or smell, runny nose, nasal congestion, muscle aches, and difficulty breathing (shortness of breath). Symptoms of influenza include, but are not limited to, fever, chills, muscle aches, cough, congestion, runny nose, headaches, and generalized fatigue. In some embodiments, the subject is asymptomatic, but has had contact within the past 14 days with a person that has tested positive for the virus.

### Lysis of Sample

In some embodiments, lysis is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. Non-limiting examples of suitable enzymes include lysozyme, lysostaphin, zymolase, cellulose, protease, and glycanase. In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40.

In some cases, at least one of the one or more lysis reagents is in solid form (e.g., lyophilized, dried, crystallized, air jetted). In some cases, all of the one or more lysis reagents are in solid form (e.g., lyophilized, dried, crystallized, air jetted). In certain embodiments, one or more lysis reagents are in the form of a lysis pellet or tablet. The lysis pellet or tablet may comprise any lysis reagent described herein. In certain embodiments, the lysis pellet or tablet may comprise one or more additional reagents (e.g., reagents to reduce or eliminate cross contamination). In a particular, non-limiting embodiment, a lysis pellet or tablet comprises Thermolabile Uracil-DNA Glycosylase (UDG) (e.g., at a concentration of about 0.02 U/uL) and murine RNAse inhibitor (e.g., at a concentration of about 1 U/uL).

In some embodiments, the lysis pellet or tablet is shelf stable for a relatively long period of time. In certain embodiments, the lysis pellet or tablet is shelf stable for at least 1 month, at least 3 months, at least 6 months, at least 9 months, at least 1 year, at least 2 years, at least 3 years, at least 4 years, at least 5 years, at least 6 years, at least 7 years, at least 8 years, at least 9 years, or at least 10 years. In some embodiments, the lysis pellet or tablet is shelf stable for 1-3 months, 1-6 months, 1-9 months, 1 month to 1 year, 1 month to 2 years, 1 month to 5 years, 1 month to 10 years, 3-6 months, 3-9 months, 3 months to 1 year, 3 months to 2 years, 3 months to 5 years, 3 months to 10 years, 6-9 months, 6 months to 1 year, 6 months to 2 years, 6 months to 5 years, 6 months to 10 years, 9 months to 1 year, 9 months to 2 years, 9 months to 5 years, 9 months to 10 years, 1-2 years, 1-3 years, 1-4 years, 1-5 years, 1-6 years, 1-7 years, 1-8 years, 1-9 years, 1-10 years, 2-5 years, 2-10 years, 3-5 years, 3-10 years, 4-10 years, 5-10 years, 6-10 years, 7-10 years, 8-10 years, or 9-10 years.

In some embodiments, the lysis pellet or tablet is thermostabilized and is stable across a wide range of temperatures. In some embodiments, the lysis pellet or tablet is stable at a temperature of at least 0° C., at least 10° C., at least 20° C., at least 37° C., at least 40° C., at least 50° C., at least 60° C., at least 65° C., at least 70° C., at least 80° C., at least 90° C., or at least 100° C. In some embodiments, the lysis pellet or tablet is stable at a temperature in a range from 0° C. to 10° C., 0° C. to 20° C., 0° C. to 37° C., 0° C. to 40° C., 0° C. to 50° C., 0° C. to 60° C., 0° C. to 65° C., 0° C. to 70° C., 0° C. to 80° C., 0° C. to 90° C., 0° C. to 100° C., 10° C. to 20° C., 10° C. to 37° C., 10° C. to 40° C., 10° C. to 50° C., 10° C. to 60° C., 10° C. to 65° C., 10° C. to 70° C., 10° C. to 80° C., 10° C. to 90° C., 10° C. to 100° C., 20° C. to 37° C., 20° C. to 40° C., 20° C. to 50° C., 20° C. to 60° C., 20° C. to 65° C., 20° C. to 70° C., 20° C. to 80° C., 20° C. to 90° C., 20° C. to 100° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

In some embodiments, the one or more lysis reagents are active at approximately room temperature (e.g., 20° C.-25° C.). In some embodiments, the one or more lysis reagents are active at elevated temperatures (e.g., at least 37° C., at least 40° C., at least 50° C., at least 60° C., at least 65° C., at least 70° C., at least 80° C., at least 90° C.). In some embodiments, chemical lysis is performed at a temperature in a range from 20° C. to 25° C., 20° C. to 30° C., 20° C. to 37° C., 20° C. to 50° C., 20° C. to 60° C., 20° C. to 65° C., 20° C. to 70° C., 20° C. to 80° C., 20° C. to 90° C., 25° C. to 30° C., 25° C. to 37° C., 25° C. to 50° C., 25° C. to 60° C., 25° C. to 65° C., 25° C. to 70° C., 25° C. to 80° C., 25° C. to 90° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

In some embodiments, cell lysis is accomplished by applying heat to a sample (thermal lysis). In certain instances, thermal lysis is performed by applying a lysis heating protocol comprising heating the sample at one or more temperatures for one or more time periods using any heater described herein. In some embodiments, a lysis heating protocol comprises heating the sample at a first temperature for a first time period. In certain instances, the first temperature is at least 37° C., at least 50° C., at least 60° C., at least 65° C., at least 70° C., at least 80° C., or at least 90° C. In certain instances, the first temperature is in a range from 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the first time period is at least 1 minute, at least 2 minutes, at least 3 minutes, at least 4 minutes, at least 5 minutes, at least 10 minutes, at least 15 minutes, at least 20 minutes, or at least 30 minutes. In certain instances, the first time period is in a range from 1 to 3 minutes, 1 to 5 minutes, 1 to 10 minutes, 1 to 15 minutes, 1 to 20 minutes, 1 to 30 minutes, 3 to 5 minutes, 3 to 10 minutes, 3 to 15 minutes, 3 to 20 minutes, 3 to 30 minutes, 5 to 10 minutes, 5 to 15 minutes, 5 to 20 minutes, 5 to 30 minutes, 10 to 20 minutes, 10 to 30 minutes, or 20 to 30 minutes. In some embodiments, a lysis heating protocol comprises heating the sample at a second temperature for a second time period. In certain instances, the second temperature is at least 37° C., at least 50° C., at least 60° C., at least 65° C., at least 70° C., at least 80° C., or at least 90° C. In certain instances, the second temperature is in a range from 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second time period is at least 1 minute, at least 2 minutes, at least 3 minutes, at least 4 minutes, at least 5 minutes, at least 10 minutes, at least 15 minutes, at least 20 minutes, or at least 30 minutes. In certain instances, the second time period is in a range from 1 to 3 minutes, 1 to 5 minutes, 1 to 10 minutes, 1 to 15 minutes, 1 to 20 minutes, 1 to 30 minutes, 3 to 5 minutes, 3 to 10 minutes, 3 to 15 minutes, 3 to 20 minutes, 3 to 30 minutes, 5 to 10 minutes, 5 to 15 minutes, 5 to 20 minutes, 5 to 30 minutes, 10 to 20 minutes, 10 to 30 minutes, or 20 to 30 minutes. In a particular, non-limiting embodiment, the first temperature is in a range from 37° C. to 50° C. (e.g., about 37° C.) and the first time period is in a range from 1 minute to 5 minutes (e.g., about 3 minutes), and the second temperature is in a range from 60° C. to 70° C. (e.g., about 65° C.) and the second time period is in a range from 5 minutes to 15 minutes (e.g., about 10 minutes). In some embodiments, a lysis heating protocol may comprise heating a sample at one or more additional temperatures for one or more additional time periods.

### Nucleic Acid Amplification

Following lysis, one or more target nucleic acids (e.g., a nucleic acid of a target pathogen) may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification.

In some embodiments, reverse transcription is performed by exposing lysate to one or more reverse transcription reagents. In certain instances, the one or more reverse transcription reagents comprise a reverse transcriptase, a DNA-dependent polymerase, and/or a ribonuclease (RNase). A reverse transcriptase generally refers to an enzyme that transcribes RNA to complementary DNA (cDNA) by polymerizing deoxyribonucleotide triphosphates (dNTPs). An RNase generally refers to an enzyme that catalyzes the degradation of RNA. In some cases, an RNase may be used to digest RNA from an RNA-DNA hybrid.

In some embodiments, DNA may be amplified according to any nucleic acid amplification method known in the art. In some embodiments, the nucleic acid amplification method is an isothermal amplification method. Isothermal amplification methods include, but are not limited to, loop-mediated isothermal amplification (LAMP), recombinase polymerase amplification (RPA), nicking enzyme amplification reaction (NEAR), nucleic acid sequence-based amplification (NASBA), strand displacement amplification (SDA), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In one embodiment, the nucleic acid amplification method is loop-mediated isothermal amplification (LAMP). In another embodiment, the nucleic acid amplification method is recombinase polymerase amplification (RPA). In another embodiment, the nucleic acid amplification method is nicking enzyme amplification reaction.

In some embodiments, the isothermal amplification methods described below include a modified nucleotide, for example, deoxyuridine triphosphate (dUTP), during amplification. In such embodiments, a subsequent test may comprise a uracil-DNA glycosylase (UDG) treatment prior to the amplification step, followed by a heat inactivation step (e.g., 95° C. for 5 minutes) (Hsieh et al., Chem Comm, 2014, 50: 3747-3749). In some embodiments, the heat inactivation step may correspond to a thermal lysis step.

Without wishing to be bound by a particular theory, it is thought that the addition of dUTP during the amplification process will reduce or eliminate potential contamination between samples. In the absence of adding dUTP and UDG, amplicons may aerosolize and contaminate future tests, potentially result in false positive test results. The use of UDG (thermolabile Uracil DNA glycosylase) generally prevents carryover contamination by specifically degrading products have already been amplified (i.e., amplicons), leaving the unamplified (new) sample untouched and ready for amplification. Using this method, tests may be performed sequentially in the same tube and/or in the same area.

In some cases, at least one of the one or more amplification reagents is in solid form (e.g., lyophilized, dried, crystallized, air jetted). In some cases, all of the one or more amplification reagents are in solid form (e.g., lyophilized, dried, crystallized, air jetted). In certain embodiments, one or more amplification reagents are in the form of an amplification pellet or tablet. The amplification pellet or tablet may comprise any amplification reagent described herein.

In some embodiments, the amplification pellet or tablet is shelf stable for a relatively long period of time. In certain embodiments, the amplification pellet or tablet is shelf stable for at least 1 month, at least 3 months, at least 6 months, at least 9 months, at least 1 year, at least 2 years, at least 3 years, at least 4 years, at least 5 years, at least 6 years, at least 7 years, at least 8 years, at least 9 years, or at least 10 years. In some embodiments, the amplification pellet or tablet is shelf stable for 1-3 months, 1-6 months, 1-9 months, 1 month to 1 year, 1 month to 2 years, 1 month to 5 years, 1 month to 10 years, 3-6 months, 3-9 months, 3 months to 1 year, 3 months to 2 years, 3 months to 5 years, 3 months to 10 years, 6-9 months, 6 months to 1 year, 6 months to 2 years, 6 months to 5 years, 6 months to 10 years, 9 months to 1 year, 9 months to 2 years, 9 months to 5 years, 9 months to 10 years, 1-2 years, 1-3 years, 1-4 years, 1-5 years, 1-6 years, 1-7 years, 1-8 years, 1-9 years, 1-10 years, 2-5 years, 2-10 years, 3-5 years, 3-10 years, 4-10 years, 5-10 years, 6-10 years, 7-10 years, 8-10 years, or 9-10 years.

In some embodiments, the amplification pellet or tablet is thermostabilized and is stable across a wide range of temperatures. In some embodiments, the amplification pellet or tablet is stable at a temperature of at least 0° C., at least 10° C., at least 20° C., at least 37° C., at least 40° C., at least 50° C., at least 60° C., at least 65° C., at least 70° C., at least 80° C., at least 90° C., or at least 100° C. In some embodiments, the amplification pellet or tablet is stable at a temperature in a range from 0° C. to 10° C., 0° C. to 20° C., 0° C. to 37° C., 0° C. to 40° C., 0° C. to 50° C., 0° C. to 60° C., 0° C. to 65° C., 0° C. to 70° C., 0° C. to 80° C., 0° C. to 90° C., 0° C. to 100° C., 10° C. to 20° C., 10° C. to 37° C., 10° C. to 40° C., 10° C. to 50° C., 10° C. to 60° C., 10° C. to 65° C., 10° C. to 70° C., 10° C. to 80° C., 10° C. to 90° C., 10° C. to 100° C., 20° C. to 37° C., 20° C. to 40° C., 20° C. to 50° C., 20° C. to 60° C., 20° C. to 65° C., 20° C. to 70° C., 20° C. to 80° C., 20° C. to 90° C., 20° C. to 100° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

In some embodiments, an isothermal amplification method described herein comprises applying heat to a sample. In certain instances, an amplification method comprises applying an amplification heating protocol comprising heating the sample at one or more temperatures for one or more time periods using any heater described herein. In some embodiments, an amplification heating protocol comprises heating the sample at a first temperature for a first time period. In certain instances, the first temperature is at least 30° C., at least 32° C., at least 37° C., at least 50° C., at least 60° C., at least 65° C., at least 70° C., at least 80° C., or at least 90° C. In certain instances, the first temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the first time period is at least 1 minute, at least 2 minutes, at least 3 minutes, at least 4 minutes, at least 5 minutes, at least 10 minutes, at least 15 minutes, at least 20 minutes, or at least 30 minutes. In certain instances, the first time period is in a range from 1 to 3 minutes, 1 to 5 minutes, 1 to 10 minutes, 1 to 15 minutes, 1 to 20 minutes, 1 to 30 minutes, 3 to 5 minutes, 3 to 10 minutes, 3 to 15 minutes, 3 to 20 minutes, 3 to 30 minutes, 5 to 10 minutes, 5 to 15 minutes, 5 to 20 minutes, 5 to 30 minutes, 10 to 20 minutes, 10 to 30 minutes, or 20 to 30 minutes.

In some embodiments, an amplification heating protocol comprises heating the sample at a second temperature for a second time period. In certain instances, the second temperature is at least 30° C., at least 32° C., at least 37° C., at least 50° C., at least 60° C., at least 65° C., at least 70° C., at least 80° C., or at least 90° C. In certain instances, the second temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second time period is at least 1 minute, at least 2 minutes, at least 3 minutes, at least 4 minutes, at least 5 minutes, at least 10 minutes, at least 15 minutes, at least 20 minutes, at least 30 minutes, at least 45 minutes, or at least 60 minutes. In certain instances, the second time period is in a range from 1 to 3 minutes, 1 to 5 minutes, 1 to 10 minutes, 1 to 15 minutes, 1 to 20 minutes, 1 to 30 minutes, 1 to 45 minutes, 1 to 60 minutes, 3 to 5 minutes, 3 to 10 minutes, 3 to 15 minutes, 3 to 20 minutes, 3 to 30 minutes, 3 to 45 minutes, 3 to 60 minutes, 5 to 10 minutes, 5 to 15 minutes, 5 to 20 minutes, 5 to 30 minutes, 5 to 45 minutes, 5 to 60 minutes, 10 to 20 minutes, 10 to 30 minutes, 10 to 45 minutes, 10 to 60 minutes, 20 to 30 minutes, 20 to 45 minutes, 20 to 60 minutes, 30 to 45 minutes, 30 to 60 minutes, or 45 to 60 minutes.

In some embodiments, an amplification heating protocol comprises heating the sample at a third temperature for a third time period. In certain instances, the third temperature is at least 30° C., at least 32° C., at least 37° C., at least 50° C., at least 60° C., at least 65° C., at least 70° C., at least 80° C., or at least 90° C. In certain instances, the third temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the third time period is at least 1 minute, at least 2 minutes, at least 3 minutes, at least 4 minutes, at least 5 minutes, at least 10 minutes, at least 15 minutes, at least 20 minutes, at least 30 minutes, at least 45 minutes, or at least 60 minutes. In certain instances, the third time period is in a range from 1 to 3 minutes, 1 to 5 minutes, 1 to 10 minutes, 1 to 15 minutes, 1 to 20 minutes, 1 to 30 minutes, 1 to 45 minutes, 1 to 60 minutes, 3 to 5 minutes, 3 to 10 minutes, 3 to 15 minutes, 3 to 20 minutes, 3 to 30 minutes, 3 to 45 minutes, 3 to 60 minutes, 5 to 10 minutes, 5 to 15 minutes, 5 to 20 minutes, 5 to 30 minutes, 5 to 45 minutes, 5 to 60 minutes, 10 to 20 minutes, 10 to 30 minutes, 10 to 45 minutes, 10 to 60 minutes, 20 to 30 minutes, 20 to 45 minutes, 20 to 60 minutes, 30 to 45 minutes, 30 to 60 minutes, or 45 to 60 minutes.

In some embodiments, a lysis heating protocol may comprise heating a sample at one or more additional temperatures for one or more additional time periods.

### LAMP

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence.

In some embodiments, the LAMP reagents comprise four or more primers. In certain embodiments, the four or more primers comprise a forward inner primer (FIP), a backward inner primer (BIP), a forward outer primer (F3), and a backward outer primer (B3). In some cases, the four or more primers target at least six specific regions of a target gene. In some embodiments, the LAMP reagents further comprise a forward loop primer (Loop F or LF) and a backward loop primer (Loop B or LB). In certain cases, the loop primers target cyclic structures formed during amplification and can accelerate amplification.

Methods of designing LAMP primers are known in the art. In some cases, LAMP primers may be designed for each target nucleic acid a diagnostic device is configured to detect. For example, a diagnostic device configured to detect a first target nucleic acid (e.g., a nucleic acid of SARS-CoV-2) and a second target nucleic acid (e.g., a nucleic acid of an influenza virus) may comprise a first set of LAMP primers directed to the first target nucleic acid and a second set of LAMP primers directed to the second target nucleic acid. In some embodiments, the LAMP primers may be designed by alignment and identification of conserved sequences in a target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art.

In certain embodiments, the target pathogen is SARS-CoV-2. In some cases, primers for amplification of a SARS-CoV-2 nucleic acid sequence are selected from regions of the virus's nucleocapsid (N) gene, envelope (E) gene, membrane (M) gene, and/or spike (S) gene. In some instances, primers were selected from regions of the SARS-CoV-2 nucleocapsid (N) gene to maximize inclusivity across known SARS-CoV-2 strains and minimize cross-reactivity with related viruses and genomes that may be presence in the sample. Exemplary LAMP primers for detection of a SARS-CoV-2 nucleic acid sequence are provided in Table 1 below.

TABLE 1
Exemplary LAMP Primers (SARS-CoV-2)

| | | SEQ |
|---|---|---|
| | | ID |
| Primer | Sequence (5′ to 3′) | NO: |
| F3_Set1 | CGGTGGACAAATTGTCAC | 1 |
| B3_Set1 | CTTCTCTGGATTTAACACACTT | 2 |
| Loop F_Set1 | TTACAAGCTTAAAGAATGTCTGAACACT | 3 |
| Loop B_Set1 | TTGAATTTAGGTGAAACATTTGTCACG | 4 |
| FIP1_Set1 | TCAGCACACAAAGCCAAAAATTTATCTGTG | 5 |
| | CAAAGGAAATTAAGGAG | |
| BIP2_Set1 | TATTGGTGGAGCTAAACTTAAAGCCCTGTA | 6 |
| | CAATCCCTTTGAGTG | |
| FIP2_Set1 | TCAGCACACAAAGCCAAAAATTTATTTTTC | 7 |
| | TGTGCAAAGGAAATTAAGGAG | |
| BIP2_Set1 | TATTGGTGGAGCTAAACTTAAAGCCTTTTC | 8 |
| | TGTACAATCCCTTTGAGTG | |
| F3_Set2 | TGCTTCAGTCAGCTGATG | 9 |
| B3_Set2 | TTAAATTGTCATCTTCGTCCTT | 10 |
| FIP_Set2 | TCAGTACTAGTGCCTGTGCCCACAATCGTT | 11 |
| | TTTAAACGGGT | |
| BIP_Set2 | TCGTATACAGGGCTTTTGACATCTATCTTG | 12 |
| | GAAGCGACAACAA | |
| Loop F_Set2 | CTGCACTTACACCGCAA | 13 |
| Loop B_Set2 | GTAGCTGGTTTTGCTAAATTCC | 14 |

In some embodiments, the LAMP reagents comprise a FIP and a BIP for one or more target nucleic acids. In some embodiments, the FIP and BIP each have a sequence that is at least 90%, at least 95%, at least 96%, at least 97%, at least 98%, at least 99%, at least 99.5%, or 100% identical to a primer sequence provided in Table 1. In some embodiments, the concentrations of FTP and BIP are each at least 0.5 μM, at least 0.6 μM, at least 0.7 μM, at least 0.8 μM, at least 0.9 μM, at least 1.0 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, or at least 2.0 μM. In some embodiments, the concentrations of FTP and BIP are each in a range from 0.5 μM to 1 μM, 0.5 μM to 1.5 μM, 0.5 μM to 2.0 μM, 1 μM to 1.5 μM, 1 μM to 2 μM, or 1.5 μM to 2 μM.

In some embodiments, the LAMP reagents comprise an F3 primer and a B3 primer for one or more target nucleic acids. In some embodiments, the F3 primer and the B3 primer each have a sequence that is at least 90%, at least 95%, at least 96%, at least 97%, at least 98%, at least 99%, at least 99.5%, or 100% identical to a primer sequence provided in Table 1. In some embodiments, the concentrations of the F3 primer and the B3 primer are each at least 0.05 μM, at least 0.1 μM, at least 0.15 μM, at least 0.2 μM, at least 0.25 μM, at least 0.3 μM, at least 0.35 μM, at least 0.4 μM, at least 0.45 μM, or at least 0.5 μM. In some embodiments, the concentrations of the F3 primer and the B3 primer are each in a range from 0.05 μM to 0.1 μM, 0.05 μM to 0.2 μM, 0.05 μM to 0.3 μM, 0.05 μM to 0.4 μM, 0.05 μM to 0.5 μM, 0.1 μM to 0.2 μM, 0.1 μM to 0.3 μM, 0.1 μM to 0.4 μM, 0.1 μM to 0.5 μM, 0.2 μM to 0.3 μM, 0.2 μM to 0.4 μM, 0.2 μM to 0.5 μM, 0.3 μM to 0.4 μM, 0.3 μM to 0.5 μM, or 0.4 μM to 0.5 μM.

In some embodiments, the LAMP reagents comprise a forward loop primer and a backward loop primer for one or more target nucleic acids. In some embodiments, the forward loop primer and the backward loop primer each have a sequence that is at least 90%, at least 95%, at least 96%, at least 97%, at least 98%, at least 99%, at least 99.5%, or 100% identical to a primer sequence provided in Table 1. In some embodiments, the concentrations of the forward loop primer and the backward loop primer are each at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, or at least 1.0 μM. In some embodiments, the concentrations of the forward loop primer and the backward loop primer are each in a range from 0.1 μM to 0.2 μM, 0.1 μM to 0.5 μM, 0.1 μM to 0.8 μM, 0.1 μM to 1.0 μM, 0.2 μM to 0.5 μM, 0.2 μM to 0.8 μM, 0.2 μM to 1.0 μM, 0.3 μM to 0.5 μM, 0.3 μM to 0.8 μM, 0.3 μM to 1.0 μM, 0.4 μM to 0.8 μM, 0.4 μM to 1.0 μM, 0.5 μM to 0.8 μM, 0.5 μM to 1.0 μM, or 0.8 μM to 1.0 μM.

In some embodiments, the LAMP reagents comprise LAMP primers designed to amplify a human or animal nucleic acid that is not associated with a pathogen, a cancer cell, or a contaminant. In some such embodiments, the human or animal nucleic acid may act as a control. For example, successful amplification and detection of the control nucleic acid may indicate that a sample was properly collected and the diagnostic test was properly run.

In some embodiments, the control nucleic acid is a nucleic acid sequence encoding human RNase P. Exemplary LAMP primers for RNase P are shown in Table 2. In some instances, the one or more LAMP reagents comprise at least four primers that each have a sequence that is at least 90%, at least 95%, at least 96%, at least 97%, at least 98%, at least 99%, at least 99.5%, or 100% identical to a primer sequence provided in Table 2.

TABLE 2
Exemplary RNase P Primers

| | | SEQ |
|---|---|---|
| | | ID |
| Primer | Sequence (5′ to 3′) | NO: |
| F3 | TTGATGAGCTGGAGCCA | 15 |
| B3 | CACCCTCAATGCAGAGTC | 16 |
| FIP | GTGTGACCCTGAAGACTCGGTTTTAGCC | 17 |
| | ACTGACTCGGATC | |
| BIP | CCTCCGTGATATGGCTCTTCGTTTTTTT | 18 |
| | CTTACATGGCTCTGGTC | |
| Loop F | HEX-ATGTGGATGGCTGAGTTGTT | 19 |
| Loop B | CATGCTGAGTACTGGACCTC | 20 |
| Quencher | CAGCCATCCACAT-BHQ1 | 21 |

In some embodiments, one or more LAMP primers comprise a label. Non-limiting examples of suitable labels include biotin, streptavidin, fluorescein isothiocyanate (FITC), fluorescein amidite (FAM), fluorescein, and digoxigenin (DIG). In some cases, labeling one or more LAMP primers may result in labeled amplicons, which may facilitate detection (e.g., via a lateral flow assay). In certain embodiments, the label is a fluorescent label. In some instances, the fluorescent label is associated with a quenching moiety that prevents the fluorescent label from signaling until the quenching moiety is removed. In certain embodiments, a LAMP primer is labeled with two or more labels.

In some embodiments, the LAMP reagents comprise a DNA polymerase with high strand displacement activity. Non-limiting examples of suitable DNA polymerases include a DNA polymerase long fragment (LF) of a thermophilic bacteria, such as Bacillus stearothermophilus (Bst), Bacillus Smithii (Bsm), Geobacillus sp. M (GspM), or Thermodesulfatator indicus (Tin), or a Taq DNA polymerase. In certain embodiments, the DNA polymerase is Bst LF DNA polymerase, GspM LF DNA polymerase, GspSSD LF DNA polymerase, Tin exo-LF DNA polymerase, or SD DNA polymerase. In each case, the DNA polymerase may be a wild type or mutant polymerase.

In some embodiments, the concentration of the DNA polymerase is at least 0.1 U/μL, at least 0.2 U/μL, at least 0.3 U/μL, at least 0.4 U/μL, at least 0.5 U/μL, at least 0.6 U/μL, at least 0.7 U/μL, at least 0.8 U/μL, at least 0.9 U/μL, or at least 1.0 U/μL. In some embodiments, the concentration of the DNA polymerase is in a range from 0.1 U/μL to 0.5 U/μL, 0.1 U/μL to 1.0 U/μL, 0.2 U/μL to 0.5 U/μL, 0.2 U/μL to 1.0 U/μL, or 0.5 U/μL to 1.0 U/μL.

In some embodiments, the LAMP reagents comprise deoxyribonucleotide triphosphates (“dNTPs”). In certain embodiments, the LAMP reagents comprise deoxyadenosine triphosphate (“dATP”), deoxyguanosine triphosphate (“dGTP”), deoxycytidine triphosphate (“dCTP”), and deoxythymidine triphosphate (“dTTP”). In certain embodiments, the concentration of each dNTP (i.e., dATP, dGTP, dCTP, dTTP) is at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the LAMP reagents comprise magnesium sulfate (MgSO₄). In certain embodiments, the concentration of MgSO₄ is at least 1 mM, at least 2 mM, at least 3 mM, at least 4 mM, at least 5 mM, at least 6 mM, at least 7 mM, at least 8 mM, at least 9 mM, or at least 10 mM. In certain embodiments, the concentration of MgSO₄ is in a range from 1 mM to 2 mM, 1 mM to 5 mM, 1 mM to 8 mM, 1 mM to 10 mM, 2 mM to 5 mM, 2 mM to 8 mM, 2 mM to 10 mM, 5 mM to 8 mM, 5 mM to 10 mM, or 8 mM to 10 mM.

In some embodiments, the LAMP reagents comprise betaine. In certain embodiments, the concentration of betaine is at least 0.1 M, at least 0.2 M, at least 0.3 M, at least 0.4 M, at least 0.5 M, at least 0.6 M, at least 0.7 M, at least 0.8 M, at least 0.9 M, at least 1.0 M, at least 1.1 M, at least 1.2 M, at least 1.3 M, at least 1.4 M, or at least 1.5 M. In certain embodiments, the concentration of betaine is in a range from 0.1 M to 0.2 M, 0.1 M to 0.5 M, 0.1 M to 0.8 M, 0.1 M to 1.0 M, 0.1 M to 1.2 M, 0.1 M to 1.5 M, 0.2 M to 0.5 M, 0.2 M to 0.8 M, 0.2 M to 1.0 M, 0.2 M to 1.2 M, 0.2 M to 1.5 M, 0.5 M to 0.8 M, 0.5 M to 1.0 M, 0.5 M to 1.2 M, 0.5 M to 1.5 M, 0.8 M to 1.0 M, 0.8 M to 1.2 M, 0.8 M to 1.5 M, 1.0 M to 1.2 M, or 1.0 M to 1.5 M.

### RPA

In some embodiments, the nucleic acid amplification reagents are RPA reagents. RPA generally refers to a method of amplifying a target nucleic acid using a recombinase, a single-stranded DNA binding protein, and a strand-displacing polymerase.

In some embodiments, the RPA reagents comprise a probe, a forward primer, and a reverse primer. The probe, forward primer, and reverse primer may be designed for each target nucleic acid a diagnostic device is configured to detect. In certain embodiments, each primer comprises at least 15 base pairs, at least 20 base pairs, at least 25 base pairs, at least 30 base pairs, at least 35 base pairs, at least 40 base pairs, at least 45 base pairs, or at least 50 base pairs. In certain embodiments, each primer comprises 15-20 base pairs, 15-30 base pairs, 15-40 base pairs, 15-50 base pairs, 20-30 base pairs, 20-40 base pairs, 20-50 base pairs, 30-40 base pairs, 30-50 base pairs, or 40-50 base pairs. In some embodiments, each primer does not have any mismatches within 3 base pairs of its 3′ terminus. In some embodiments, each primer comprises 10 or fewer, 9 or fewer, 8 or fewer, 7 or fewer, 6 or fewer, 5 or fewer, 4 or fewer, 3 or fewer, 2 or fewer, 1 or fewer, or no mismatches. In some embodiments, each mismatch is at least 3 base pairs, at least 4 base pairs, at least 5 base pairs, at least 6 base pairs, at least 7 base pairs, at least 8 base pairs, at least 9 base pairs, or at least 10 base pairs from the 3′ terminus. While mismatches more than 3 base pairs away from the 3′ terminus of the primer have been found to be well tolerated in RPA, multiple mismatches within 3 base pairs of the 3′ terminus have been found to inhibit the reaction.

As an illustrative example, in some instances, a first target nucleic acid is a nucleic acid of SARS-CoV-2. Exemplary RPA primers for detection of a nucleic acid sequence from the SARS-CoV-2 nucleocapsid (N) gene are provided in Table 3 below.

TABLE 3
Exemplary Recombination Polymerase
Amplification Primers

| | | SEQ |
|---|---|---|
| | | ID |
| RPA_primer | Sequence | NO: |
| Forward | GTACTGCCACTAAAGCATACAATGTAACAC | 22 |
| Primer | | |
| Reverse | {6-FAM}AATATGCTTATTCAGCAAAATGA | 23 |
| Primer | CTTGATCT | |
| Probe | {biotin}CAGACAAGGAACTGATTACAA | 24 |
| | ACATTGGCCGCA{dSpacer}ATTGCACAA | |
| | TTTGCC{phos} | |

In some embodiments, the RPA reagents comprise a forward primer. In certain embodiments, the forward primer is at least 70%, at least 75%, at least 80%, at least 85%, at least 90%, at least 95%, at least 99%, or 100% identical to SEQ ID NO: 22. In some embodiments, the forward primer is at least 1 base pair, at least 2 base pairs, at least 3 base pairs, at least 4 base pairs, or at least 5 base pairs longer or shorter than SEQ ID NO: 22. In some embodiments, the forward primer comprises an antigenic tag. In certain embodiments, the concentration of the forward primer is at least 100 nM, at least 200 nM, at least 300 nM, at least 400 nM, at least 500 nM, at least 600 nM, at least 700 nM, at least 800 nM, at least 900 nM, or at least 1000 nM. In certain embodiments, the concentration of the forward primer is in a range from 100 nM to 200 nM, 100 nM to 500 nM, 100 nM to 800 nM, 100 nM to 1000 nM, 200 nM to 500 nM, 200 nM to 800 nM, 200 nM to 1000 nM, 500 nM to 800 nM, 500 nM to 1000 nM, or 800 nM to 1000 nM.

In some embodiments, the RPA reagents comprise a reverse primer. In certain embodiments, the reverse primer is at least 70%, at least 75%, at least 80%, at least 85%, at least 90%, at least 95%, at least 99%, or 100% identical to SEQ ID NO: 23. In some embodiments, the reverse primer is at least 1 base pair, at least 2 base pairs, at least 3 base pairs, at least 4 base pairs, or at least 5 base pairs longer or shorter than SEQ ID NO: 23. In some embodiments, the reverse primer comprises an antigenic tag. In certain embodiments, the concentration of the reverse primer is at least 100 nM, at least 200 nM, at least 300 nM, at least 400 nM, at least 500 nM, at least 600 nM, at least 700 nM, at least 800 nM, at least 900 nM, or at least 1000 nM. In certain embodiments, the concentration of the reverse primer is in a range from 100 nM to 200 nM, 100 nM to 500 nM, 100 nM to 800 nM, 100 nM to 1000 nM, 200 nM to 500 nM, 200 nM to 800 nM, 200 nM to 1000 nM, 500 nM to 800 nM, 500 nM to 1000 nM, or 800 nM to 1000 nM.

In some embodiments, the RPA reagents further comprises a probe. In certain embodiments, the probe is at least 70%, at least 75%, at least 80%, at least 85%, at least 90%, at least 95%, at least 99%, or 100% identical to SEQ ID NO: 24. In some embodiments, the concentration of the probe is at least 50 nM, at least 60 nM, at least 70 nM, at least 80 nM, at least 90 nM, at least 100 nM, at least 110 nM, at least 120 nM, at least 130 nM, at least 140 nM, at least 150 nM, at least 160 nM, at least 170 nM, at least 180 nM, at least 190 nM, or at least 200 nM. In some embodiments, the concentration of the probe is in a range from 50 nM to 100 nM, 50 nM to 120 nM, 50 nM to 150 nM, 50 nM to 180 nM, 50 nM to 200 nM, 100 nM to 120 nM, 100 nM to 150 nM, 100 nM to 180 nM, 100 nM to 200 nM, 120 nM to 180 nM, 120 nM to 200 nM, or 150 nM to 200 nM.

In some embodiments, the RPA reagents comprise RPA primers designed to amplify a human or animal nucleic acid that is not associated with a pathogen, a cancer cell, or a contaminant. In some such embodiments, the human or animal nucleic acid may act as a control. For example, successful amplification and detection of the control nucleic acid may indicate that the diagnostic test was properly run (e.g., sample was collected, cells were lysed, nucleic acids were amplified). On the other hand, failure to detect the control nucleic acid may indicate one or more of the following: improper specimen collection resulting in the lack of sufficient human sample material, improper extraction of nucleic acid from the sample, ineffective inhibition of RNAse in the sample, improper assay set up and execution, and reagent or equipment malfunction.

In some instances, the control nucleic acid is a nucleic acid sequence encoding human RNase P. In some embodiments, the RPA reagents comprise primers (e.g., forward primers, reverse primers) and probes configured to detect a nucleic acid sequence encoding human RNase P.

In some embodiments, the RPA reagents comprise one or more recombinase enzymes. Non-limiting examples of suitable recombinase enzymes include T4 UvsX protein and T4 UvsY protein. In some embodiments, the concentration of each recombinase enzyme is at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.10 mg/mL, at least 0.11 mg/mL, at least 0.12 mg/mL, at least 0.13 mg/mL, at least 0.14 mg/mL, or at least 0.15 mg/mL. In some embodiments, the concentration of each recombinase enzyme is in a range from 0.01 mg/mL to 0.05 mg/mL, 0.01 mg/mL to 0.1 mg/mL, 0.01 mg/mL to 0.15 mg/mL, 0.05 mg/mL to 0.1 mg/mL, 0.05 mg/mL to 0.15 mg/mL, or 0.10 mg/mL to 0.15 mg/mL.

In some embodiments, the RPA reagents comprise one or more single-stranded DNA binding proteins. A non-limiting example of a suitable single-stranded DNA binding protein is T4 gp32 protein. In certain embodiments, the concentration of the single-stranded DNA binding protein is at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, or at least 1.0 mg/mL. In certain embodiments, the concentration of the single-stranded DNA binding protein is in a range from 0.1 mg/mL to 0.2 mg/mL, 0.1 mg/mL to 0.5 mg/mL, 0.1 mg/mL to 0.8 mg/mL, 0.1 mg/mL to 1.0 mg/mL, 0.2 mg/mL to 0.5 mg/mL, 0.2 mg/mL to 0.8 mg/mL, 0.2 mg/mL to 1.0 mg/mL, 0.5 mg/mL to 0.8 mg/mL, 0.5 mg/mL to 1.0 mg/mL, or 0.8 mg/mL to 1.0 mg/mL.

In some embodiments, the RPA agents comprise a DNA polymerase. A non-limiting example of a suitable DNA polymerase is Staphylococcus aureus DNA polymerase (Sau). In certain embodiments, the concentration of the DNA polymerase is at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, or at least 0.1 mg/mL. In certain embodiments, the concentration of the single-stranded DNA binding protein is in a range from 0.01 mg/mL to 0.02 mg/mL, 0.01 mg/mL to 0.05 mg/mL, 0.01 mg/mL to 0.08 mg/mL, 0.01 mg/mL to 0.1 mg/mL, 0.02 mg/mL to 0.05 mg/mL, 0.02 mg/mL to 0.08 mg/mL, 0.02 mg/mL to 0.1 mg/mL, 0.05 mg/mL to 0.08 mg/mL, 0.05 mg/mL to 0.1 mg/mL, or 0.08 mg/mL to 0.1 mg/mL.

In some embodiments, the RPA agents comprise an endonuclease. A non-limiting example of a suitable endonuclease is Endonuclease IV. In some embodiments, the concentration of the endonuclease is at least 0.001 mg/mL, at least 0.002 mg/mL, at least 0.003 mg/mL, at least 0.004 mg/mL, at least 0.005 mg/mL, at least 0.006 mg/mL, at least 0.007 mg/mL, at least 0.008 mg/mL, at least 0.009 mg/mL, at least 0.01 mg/mL, at least 0.02 mg/mL, or at least 0.05 mg/mL. In some embodiments, the concentration of the endonuclease is in a range from 0.001 mg/mL to 0.005 mg/mL, 0.001 mg/mL to 0.01 mg/mL, 0.001 mg/mL to 0.02 mg/mL, 0.001 mg/mL to 0.05 mg/mL, 0.005 mg/mL to 0.01 mg/mL, 0.005 mg/mL to 0.02 mg/mL, 0.005 mg/mL to 0.05 mg/mL, 0.01 mg/mL to 0.02 mg/mL, or 0.01 mg/mL to 0.05 mg/mL.

In some embodiments, the RPA reagents comprise dNTPs (e.g., dATP, dGTP, dCTP, dTTP). In certain embodiments, the concentration of each dNTP is at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.1 mM to 0.2 mM, 0.1 mM to 0.5 mM, 0.1 mM to 0.8 mM, 0.1 mM to 1.0 mM, 0.1 mM to 1.5 mM, 0.1 mM to 2.0 mM, 0.2 mM to 0.5 mM, 0.2 mM to 0.8 mM, 0.2 mM to 1.0 mM, 0.2 mM to 1.5 mM, 0.2 mM to 2.0 mM, 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the RPA reagents comprise one or more additional components. Non-limiting examples of suitable components include DL-Dithiothreitol, phosphocreatine disodium hydrate, creatine kinase, and adenosine 5′-triphosphate disodium salt.

### Nicking Enzyme Amplification Reaction (NEAR)

In some embodiments, amplification of one or more target nucleic acids is accomplished through the use of a nicking enzyme amplification reaction (NEAR) reaction. NEAR generally refers to a method for amplifying a target nucleic acid using a nicking endonuclease and a strand displacing DNA polymerase. In some cases, NEAR may allow for amplification of very small amplicons.

In some embodiments, the NEAR reagents comprise a forward template and a reverse template. In certain embodiments, the forward template comprises a nucleic acid sequence having a hybridization region at the 3′ end that is complementary to the 3′ end of a target antisense strand (e.g., an antisense sequence to the reverse-transcribed SARS-CoV-2 nucleocapsid sequence), a nicking enzyme binding site and a nicking site upstream of the hybridization region, and a stabilizing region upstream of the nicking site. In certain embodiments, the first reverse template comprises a nucleic acid sequence having a hybridization region at the 3′ end that is complementary to the 3′ end of a target gene sense strand (e.g., a SARS-CoV-2 nucleocapsid gene sense strand), a nicking enzyme binding site and a nicking site upstream of the hybridization region, and a stabilizing region upstream of the nicking site. Designs of templates suitable for NEAR methods disclosed herein are provided in, for example, U.S. Pat. Nos. 9,617,586 and 9,689,031, each of which are incorporated herein by reference.

In some embodiments, the NEAR composition further comprises a probe oligonucleotide. In certain embodiments, the probe comprises a nucleotide sequence complementary to the target gene nucleotide sequence. In some instances, for example, the probe is a SARS-CoV-2 specific probe.

In some embodiments, the probe is conjugated to a detectable label. In some embodiments, the detectable label is selected from the group consisting of a fluorophore, an enzyme, a quencher, an enzyme inhibitor, a radioactive label, a member of a binding pair, and a combination thereof. In some embodiments, one or more of the forward template and the reverse template comprises a least one modified nucleotide, spacer, or blocking group. In some embodiments, at least one modified nucleotide includes a 2′ modification.

In some embodiments, the NEAR reagents comprise a DNA polymerase. Examples of suitable DNA polymerases include, but are not limited to, Geobacillus bogazici DNA polymerase, Bst (large fragment), exo-DNA Polymerase, and Manta 1.0 DNA Polymerase (Enzymatics 3 e). In some embodiments, the NEAR reagents comprise at least one nicking enzyme. Non-limiting examples of suitable nicking enzymes include Nt. BspQI, Nb. BbvCi, Nb. BsmI, Nb. BsrDI, Nb. BtsI, Nt. AlwI, Nt. BbvCI, Nt. BstNBI, Nt. CviPII, Nb. Bpul OI, Nt. BpulOI, and N. BspD61. In some embodiments, the NEAR reagents further comprise dNTPs (e.g., dATP, dGTP, dCTP, dTTP).

In some embodiments, amplification is performed under essentially isothermal conditions.

### Molecular Switches

As described herein, a sample undergoes lysis and amplification prior to detection. The reagents associated with lysis and/or detection may be in solid form (e.g., lyophilized, dried, crystallized, air jetted). In certain embodiments, one or more (and, in some cases, all) of the reagents necessary for lysis and/or amplification are present in a single pellet or tablet. In some embodiments, a pellet or tablet may comprise two or more enzymes, and it may be necessary for the enzymes to be activated in a particular order. Therefore, in some embodiments, the enzyme tablet further comprises one or more molecular switches. Molecular switches, as described herein, are molecules that, in response to certain conditions, reversibly switch between two or more stable states. In some embodiments, the condition that causes the molecular switch to change its configuration is pH, light, temperature, an electric current, microenvironment, or the presence of ions and other ligands. In one embodiment, the condition is heat. In some embodiments, the molecular switches described herein are aptamers. Aptamers generally refer to oligonucleotides or peptides that bind to specific target molecules (e.g., the enzymes described herein). The aptamers, upon exposure to heat or other conditions, may dissociate from the enzymes. With the use of molecular switches, the processes described herein (e.g., lysis, decontamination, reverse transcription, and amplification) may be performed in a single test tube with a single enzymatic tablet.

In one illustrative embodiment, an enzymatic tablet comprises UDG, reverse transcriptase, and DNA polymerase (e.g., Bst DNA polymerase). Initially, the sample may be heated at 37° C., the temperature at which UDG is active, in order to decontaminate the sample. At 37° C., molecular switches may bind to, and inactivate, the reverse transcriptase and DNA polymerase. This may advantageously ensure that they do not interfere with the UDG decontamination reaction. Next, following decontamination, the sample may be heated at 65° C., which deactivates heat-sensitive UDG but causes the molecular switches to release, and therefore activate, the reverse transcriptase and DNA polymerase. Reverse transcription may then proceed.

Therefore, in some embodiments, the molecular switches (aptamers) specifically bind the enzymes described herein, such that the enzymes are inactivated. “Inactivated,” as used herein, refers to an enzyme that is not enzymatically active; that is, it cannot perform its enzymatic function. Aptamers, as described herein, are single-stranded nucleic acid molecules (about 5-25 kDa) having unique configurations that allow them to bind to molecular targets with high specificity and affinity. In one embodiment, the aptamers are DNA or RNA aptamers or hybrid DNA/RNA aptamers. Similar to antibodies, aptamers possess binding affinities in the low nanomolar to picomolar range.

The small size of an aptamer enhances its ability to bind to a specific site on an enzyme, altering the function of that site without affecting the functions of other sites on the enzyme. In some embodiments, the aptamers inhibit the enzymatic activity of a reverse transcriptase, a DNA polymerase (e.g., Bst DNA polymerase), and/or a glycosylase. In some embodiments, the presently disclosed methods produce at least about a 10%, 15%, 20%, 25%, 30%, 35%, 40%. 45%, 50%, 55%, 60%, 65%, 70%, 75%, 80%, 85%, 90%, 95%, or even 100% inhibition of enzymatic activity relative to enzymatic activity measured in absence of aptamers (e.g. a control) in an assay.

The term “specifically binds,” as used herein, refers to a molecule (e.g., an aptamer) that binds to a target (e.g., an enzyme) with at least five-fold greater affinity as compared to any non-targets. e.g., at least 10-, 20-, 50-, or 100-fold greater affinity.

The length of the aptamers of the presently disclosed subject matter is not limited, but typical aptamers have a length of about 10 to about 120 nucleotides, such as about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, about 105 nucleotides, about 110 nucleotides, about 115 nucleotides, about 120 nucleotides, or more nucleotides. In certain embodiments, the aptamer may have additional nucleotides attached to the 5′- and/or 3′ end.

The polynucleotide aptamers may be comprised of ribonucleotides only (RNA aptamers), deoxyribonucleotides only (DNA aptamers), or a combination of ribonucleotides and deoxyribonucleotides. The nucleotides may be naturally occurring nucleotides (e.g., ATP, TTP, GTP, CTP, UTP) or modified nucleotides. As used herein, “modified nucleotide” refers to a nucleotide comprising a base such as, for example, adenine, guanine, cytosine, thymine, and uracil, xanthine, inosine, and queuosine that has been modified by the replacement or addition of one or more atoms or groups. For example, the modification may comprise a nucleotide that is modified with respect to the base moiety, such as a/an alkylated, halogenated, thiolated, aminated, amidated, or acetylated base, in various combinations. Modified nucleotides also include nucleotides that comprise a sugar moiety modification (e.g., 2′-fluoro or 2′-O-methyl nucleotides), as well as nucleotides having sugars or analogs thereof that are not ribosyl. For example, the sugar moieties may be, or be based on, mannoses, arabinoses, glucopyranoses, galactopyranoses, 4-thioribose, and other sugars, heterocycles, or carbocycles.

### Detection

In some embodiments, amplified nucleic acids (i.e., amplicons) may be detected using any suitable methods. In some embodiments, one or more target nucleic acid sequences are detected using a lateral flow assay strip. In some embodiments, one or more target nucleic acid sequences are detected using a colorimetric assay.

In some embodiments, one or more target nucleic acid sequences are detected using a lateral flow assay strip (e.g., in a “chimney” detection component, a cartridge, a blister pack). In some embodiments, a fluidic sample (e.g., fluidic contents of a reaction tube, a reagent reservoir, and/or a blister pack chamber) is transported through the lateral flow assay strip via capillary action. In some embodiments, the fluidic sample may comprise labeled amplicons.

In some embodiments, the fluidic sample is introduced to a first sub-region (e.g., a sample pad) of the lateral flow assay strip. In certain embodiments, the fluidic sample subsequently flows through a second sub-region (e.g., a particle conjugate pad) comprising a plurality of labeled particles. In some cases, the particles comprise gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some cases, as an amplicon-containing fluidic sample flows through the second sub-region (e.g., a particle conjugate pad), a labeled nanoparticle binds to a label of an amplicon, thereby forming a particle-amplicon conjugate.

In some embodiments, the fluidic sample (e.g., comprising a particle-amplicon conjugate) subsequently flows through a third sub-region (e.g., a test pad) comprising one or more test lines. In some embodiments, a first test line comprises a capture reagent (e.g., an immobilized antibody) configured to detect a first target nucleic acid. In some embodiments, a particle-amplicon conjugate may be captured by one or more capture reagents (e.g., immobilized antibodies), and an opaque marking may appear. The marking may have any suitable shape or pattern (e.g., one or more straight lines, curved lines, dots, squares, check marks, x marks).

In certain embodiments, the lateral flow assay strip comprises one or more additional test lines. In some instances, each test line of the lateral flow assay strip is configured to detect a different target nucleic acid. In some instances, two or more test lines of the lateral flow assay strip are configured to detect the same target nucleic acid. The test line(s) may have any suitable shape or pattern (e.g., one or more straight lines, curved lines, dots, squares, check marks, x marks).

In certain embodiments, the third sub-region (e.g., the test pad) of the lateral flow assay strip further comprises one or more control lines. In certain instances, a first control line is a human (or animal) nucleic acid control line. In some embodiments, for example, the human (or animal) nucleic acid control line is configured to detect a nucleic acid (e.g., RNase P) that is generally present in all humans (or animals). In some cases, the human (or animal) nucleic acid control line becoming detectable indicates that a human (or animal) sample was successfully collected, nucleic acids from the sample were amplified, and the amplicons were transported through the lateral flow assay strip. In certain instances, a first control line is a lateral flow control line. In some cases, the lateral flow control line becoming detectable indicates that a liquid was successfully transported through the lateral flow assay strip. In some embodiments, the lateral flow assay strip comprises two or more control lines. The control line(s) may have any suitable shape or pattern (e.g., one or more straight lines, curved lines, dots, squares, check marks, x marks). In some instances, for example, the lateral flow assay strip comprises a human (or animal) nucleic acid control line and a lateral flow control line.

In certain embodiments, the lateral flow assay strip comprises a fourth sub-region (e.g., a wicking area) to absorb fluid flowing through the lateral flow assay strip. Any excess fluid may flow through the fourth sub-region.

As an illustrative example, a fluidic sample comprising an amplicon labeled with biotin and FITC may be introduced into a lateral flow assay strip (e.g., through a sample pad of a lateral flow assay strip). In some embodiments, as the labeled amplicon is transported through the lateral flow assay strip (e.g., through a particle conjugate pad of the lateral flow assay strip), a gold nanoparticle labeled with streptavidin may bind to the biotin label of the amplicon. In some cases, the lateral flow assay strip (e.g., a test pad of the lateral flow assay strip) may comprise a first test line comprising an anti-FITC antibody. In some embodiments, the gold nanoparticle-amplicon conjugate may be captured by the anti-FITC antibody, and an opaque band may develop as additional gold nanoparticle-amplicon conjugates are captured by the anti-FITC antibodies of the first test line. In some cases, the lateral flow assay strip (e.g., a test pad of the lateral flow assay strip) further comprises a first lateral flow control line comprising biotin. In some embodiments, excess gold nanoparticles labeled with streptavidin (i.e., gold nanoparticles that were not conjugated to an amplicon) transported through the lateral flow assay strip may bind to the biotin of the first lateral flow control line, demonstrating that liquid was successfully transported to the first lateral flow control line. In some embodiments, one or more target nucleic acid sequences are detected using a colorimetric assay. In certain embodiments, for example, a fluidic sample is exposed to a reagent that undergoes a color change when bound to a target nucleic acid (e.g., viral DNA or RNA), such as with an enzyme-linked immunoassay. In some embodiments, the assay further comprises a stop reagent, such as sulfonic acid. That is, when the fluidic sample is mixed with the reagents, the solution turns a specific color (e.g., red) if the target nucleic acid is present, and the sample is positive. If the solution turns a different color (e.g., green), the target nucleic acid is not present, and the sample is negative. In some embodiments, the colorimetric assay may be a colorimetric LAMP assay; that is, the LAMP reagents may react in the presence or absence of a target nucleic acid sequence (e.g., from SARS-CoV-2) to turn one of two colors.

### Isothermal Amplification CRISPR-based Detection

In some embodiments, a diagnostic method uses CRISPR/Cas detection and/or a diagnostic system comprises one or more reagents for CRISPR/Cas detection. CRISPR generally refers to Clustered Regularly Interspaced Short Palindromic Repeats, and Cas generally refers to a particular family of proteins. In some embodiments, the CRISPR/Cas detection platform can be combined with an isothermal amplification method to create a single step reaction (Joung et al., “Point-of-care testing for COVID-19 using SHERLOCK diagnostics,” 2020). For example, the amplification and CRISPR detection may be performed using reagents having compatible chemistries (e.g., reagents that do not interact detrimentally with one another and are sufficiently active to perform amplification and detection). In some embodiments, CRISPR/Cas detection is combined with LAMP.

CRISPR/Cas detection platforms are known in the art. Examples of such platforms include SHERLOCK® and DETECTR® (see, e.g., Kellner et al., Nature Protocols, 2019, 14: 2986-3012; Broughton et al., Nature Biotechnology, 2020; Joung et al., 2020).

In some embodiments, CRISPR/Cas methods are used to detect a target nucleic acid sequence (e.g., from a pathogen). In particular, a guide RNA (gRNA) designed to recognize a specific target sequence (e.g., a SARS-CoV-2-specific sequence) may be used to detect target nucleic acid sequences present in a sample. If the sample comprises the target nucleic acid sequence, the gRNA will bind the target nucleic acid sequence and activate a programmable nuclease (e.g., a Cas protein), which may then cleave a reporter molecule and release a detectable signal (e.g., a reporter molecule tagged with specific antibodies for the lateral flow test, a fluorophore, a dye, a polypeptide, or a substrate for a specific colorimetric dye). In some embodiments, the detectable moiety binds to a capture reagent (e.g., an antibody) on a lateral flow strip, as described herein.

In some embodiments, the one or more reagents for CRISPR/Cas detection comprise one or more guide nucleic acids. As noted above, a guide nucleic acid may comprise a segment with reverse complementarity to a segment of the target nucleic acid sequence. In some embodiments, the guide nucleic acid is selected from a group of guide nucleic acids that have been screened against the nucleic acid of a strain of an infection or genomic locus of interest. In certain instances, for example, the guide nucleic acid may be selected from a group of guide nucleic acids that have been screened against the nucleic acid of a strain of SARS-CoV-2. In some embodiments, guide nucleic acids that are screened against the nucleic acid of a target sequence of interest can be pooled. Without wishing to be bound by a particular theory, it is thought that pooled guide nucleic acids directed against a single target nucleic acid can ensure broad coverage of the target nucleic acid within a single reaction. The pooled guide nucleic acids, in some embodiments, are directed to different regions of the target nucleic acid and may be sequential or non-sequential.

In some embodiments, a guide nucleic acid comprises a crRNA and/or tracrRNA. The guide nucleic acid may not be naturally occurring and may be made by artificial combination of otherwise separate segments of sequence. For example, in some embodiments, an artificial guide nucleic acid may be synthesized by chemical synthesis, genetic engineering techniques, and/or artificial manipulation of isolated segments of nucleic acids. In some embodiments, the targeting region of a guide nucleic acid is at least 10, 15, 20, 25, 30, 35, 40, 45, 50, 55, or 60 nucleotides (nt) in length. In some embodiments, the targeting region of a guide nucleic acid has a length in a range from 10 to 20 nt, 10 to 30 nt, 10 to 40 nt, 10 to 50 nt, 10 to 60 nt, 20 to 30 nt, 20 to 40 nt, 20 to 50 nt, 20 to 60 nt, 30 to 40 nt, 30 to 50 nt, 30 to 60 nt, 40 to 50 nt, 40 to 60 nt, or 50 to 60 nt.

In some embodiments, the one or more reagents for CRISPR/Cas detection comprise one or more programmable nucleases. In some embodiments, a programmable nuclease is capable of sequence-independent cleavage after the gRNA binds to its specific target sequence. In some instances, the programmable nuclease is a Cas protein. Non-limiting examples of suitable Cas proteins include Cas9, Cas12a, Cas12b, Cas13, and Cas14. In general, Cas9 and Cas12 nucleases are DNA-specific, Cas13 is RNA-specific, and Cas14 targets single-stranded DNA.

In some embodiments, the one or more reagents for CRISPR/Cas detection comprise a plurality of guide nucleic acids and a plurality of programmable nucleases. In some embodiments, each guide nucleic acid of the plurality of guide nucleic acids targets a different nucleic acid and is associated with a different programmable nuclease. As an illustrative example, if a diagnostic device is configured to detect two different target nucleic acids, the one or more CRISPR/Cas reagents may comprise at least two different guide nucleic acids and at least two different programmable nucleases. If two target nucleic acids are present in a sample, then two different programmable nucleases will be activated, which will result in the release of two unique detectable moieties. Thus, in this manner, the CRISPR/Cas detection system may be used to detect more than one target nucleic acid. In some embodiments, the CRISPR/Cas detection system may be used to detect at least 2, at least 3, at least 4, at least 5, at least 6, at least 7, at least 8, at least 9, or at least 10 target nucleic acids.

### Instructions & Software

In some embodiments, a diagnostic system comprises instructions for using a diagnostic device and/or otherwise performing a diagnostic test method. The instructions may include instructions for the use, assembly, and/or storage of the diagnostic device and any other components associated with the diagnostic system. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications).

In some embodiments, the instructions are provided as part of a software-based application. In certain cases, the application can be downloaded to a smartphone or device, and then guides a user through steps to use the diagnostic device. In some embodiments, the instructions instruct a user when to add certain reagents and how to do so. For example, in certain instances, the instructions may instruct a user when to change reaction tube caps and how to release reagents from the reaction tube caps (e.g., by depressing a button, twisting a portion of the reaction tube cap, etc.). In some embodiments, the instructions instruct a user on beginning and/or ending heating protocols. In some cases, a user may receive an alert (e.g., on a mobile application) when a heating protocol (e.g., a lysis heating protocol, an amplification heating protocol) is complete. In some embodiments, the application may validate that the diagnostic test was performed correctly.

In some embodiments, a software-based application may be connected (e.g., via a wired or wireless connection) to one or more components of a diagnostic system. In certain embodiments, for example, a heater may be controlled by a software-based application. In some cases, a user may select an appropriate heating protocol through the software-based application. In some cases, an appropriate heating protocol may be selected remotely (e.g., not by the immediate user). In some cases, the software-based application may store information (e.g., regarding temperatures used during the processing steps) from the heater.

In some embodiments, a diagnostic systems comprises or is associated with software to read and/or analyze test results. In some embodiments, a device (e.g., a camera, a smartphone) is used to generate an image of a test result (e.g., one or more lines detectable on a lateral flow assay strip). In certain cases, a machine vision software application is employed to evaluate the image and provide a positive or negative test result. That result may be communicated directly to a user or to a medical professional. In some cases, the test result may be further communicated to a remote database server. In some embodiments, the remote database server stores test results as well as user information. For example, the remote database server may store at least one of name, social security number, date of birth, address, phone number, email address, medical history, and medications.

In some embodiments, the remote database server may track and monitor locations of users (e.g., using smartphones or remote devices with tracking capabilities). In some cases, the remote database server can be used to notify individuals who come into contact with or within a certain distance of any user who has tested positive for a particular illness (e.g., COVID-19). In some cases, a user's test results, information, and/or location may be communicated to state and/or federal health agencies.

In some embodiments, a user may use an electronic device (e.g., a smartphone, a tablet, a camera) to acquire an image of the visible portion of the lateral flow assay strip. In some embodiments, software running on the electronic device may be used to analyze the image (e.g., by comparing any lines or other markings that appear on the lateral flow assay strip with known patterns of markings). If the software's analysis differs from a result entered by a user, the user may be asked to double check that they entered the correct pattern, and the user may be given the opportunity

After uploading the image, a computer vision algorithm is run to electronically call the bands. If the band-pattern result determined by the algorithm differs from the band pattern result entered by the user, the user is asked to double-check that they entered the correct band-pattern, and the user is given the opportunity to redo to the “Record Results” page. Alternatively, in some embodiments, the interpretation is performed solely by the computer-vision algorithm. Based on the result that the user entered, the user is shown the corresponding “Test Complete” screen in the mobile application, which tells the user if the test result is positive, negative, or invalid. In addition to providing the test result, careful language is used to ensure that the user can properly interpret the meaning of the result.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

What is claimed is:

1. A detection component of a diagnostic test comprising:
a first reservoir for containing a first solution;
a lateral flow assay strip;
a receptacle configured to receive and fluidly connect to a second reservoir containing a sample, wherein fluidly connecting the second reservoir to the receptacle allows the sample to flow from the second reservoir to the lateral flow assay strip; and
a seal positioned between the first reservoir and the lateral flow assay strip, wherein fluidly connecting the second reservoir to the receptacle opens the seal to allow the first solution to flow from the first reservoir to the lateral flow assay strip.

2. The detection component of claim 1, wherein the seal is a frangible seal.

3. The detection component of claim 2, wherein the seal is a breakable metal foil.

4. The detection component of claim 2, wherein the seal is a breakable film.

5. The detection component of claim 4, wherein the breakable film is formed of plastic.

6. The detection component of claim 4, wherein the breakable film is formed of an elastomer.

7. The detection component of claim 1, wherein the seal is a wall of the first reservoir that is ruptured under pressure from the second reservoir.

8. The detection component of claim 1, wherein the seal is a valve.

9. The detection component of claim 8, wherein the valve is a septum valve, and wherein fluidly connecting the second reservoir with the receptacle is configured to open the septum valve.

10. The detection component of claim 1, wherein the first reservoir, the lateral flow assay strip, and the seal are disposed in a housing, and wherein the seal is positioned on the first reservoir.

11. The detection component of claim 10, wherein the housing includes a needle disposed in the first reservoir configured to puncture the second reservoir when the second reservoir is moved against the needle.

12. The detection component of claim 10, wherein the housing includes a blade configured to puncture the second reservoir when the second reservoir is moved against the blade.

13. The detection component of claim 1, wherein the first reservoir is a diluent reservoir.

14. The detection component of claim 1, wherein the seal is opened by the second reservoir before the second reservoir is fluidly connected to the receptacle.

15. The detection component of claim 1, wherein the seal is opened by the second reservoir concurrently with the second reservoir being fluidly connected to the receptacle.

16. The detection component of claim 1, wherein the seal is opened by the second reservoir after the second reservoir is fluidly connected to the receptacle.

17. The detection component of claim 1, further comprising a lever disposed in the receptacle and configured to apply force to the first reservoir when the second reservoir is fluidly connected to the receptacle.

18. The detection component of claim 1, further comprising a third reservoir fluidly connected to the lateral flow assay strip, wherein the third reservoir is configured to receive the first solution from the first reservoir when the seal is opened and the sample from the second reservoir when the second reservoir is fluidly connected to the receptacle such that the first solution and the sample may mix in the third reservoir.

19. A detection component of a diagnostic test, comprising:
a vial including an internal volume, the vial including:
a first reservoir for containing a sample,
a second reservoir for containing a first solution, and
a seal positioned between the first reservoir and the second reservoir, wherein the seal is configured to be opened to fluidly connect the first reservoir and the second reservoir;
a third reservoir configured to receive the sample and the first solution when the seal is opened;
a lateral flow assay strip fluidly connected to the third reservoir; and
an actuator configured to selectively open the seal.

20. The detection component of claim 19, further comprising a housing, wherein the housing includes a receptacle that received the vial, and wherein the actuator and the lateral flow assay strip are disposed in the housing.

21. The detection component of claim 19, wherein the second reservoir is formed as a frangible blister.

22. The detection component of claim 21, wherein the seal is a wall of the frangible blister, and wherein the seal is configured to be opened to fluidly connect the first reservoir and the third reservoir.

23. The detection component of claim 19, wherein the seal is a frangible seal.

24. The detection component of claim 23, wherein the seal is a breakable metal foil.

25. The detection component of claim 23, wherein the seal is a breakable film.

26. The detection component of claim 25, wherein the breakable film is formed of plastic.

27. The detection component of claim 25, wherein the breakable film is formed of an elastomer.

28. The detection component of claim 19, further comprising a second seal positioned between the second reservoir and the third reservoir.

29. The detection component of claim 28, wherein the seal is a first frangible seal, and the second seal is a second frangible seal.

30. The detection component of claim 28, wherein the actuator is configured to selectively open the second seal.

31. The detection component of claim 28, further comprising a second actuator configured to selectively open the second seal.

32. The detection component of claim 19, wherein the actuator is a crush effector configured to apply force to pressurize at least one of the first reservoir and the second reservoir to open the seal.

33. The detection component of claim 19, where the first solution is a diluent.

34. A method of performing a diagnostic test, comprising:
depositing a sample in a first reservoir;
moving the first reservoir into a receptacle of a detection component;
opening a seal positioned between a second reservoir containing a first solution and a lateral flow assay strip to allow the first solution to flow toward the lateral flow assay strip in response to moving the first reservoir into the receptacle; and
fluidly connecting the first reservoir with the receptacle to allow the sample to flow toward the lateral flow assay strip.

35. The method of claim 34, further comprising mixing the first solution and the sample in a third reservoir fluidly connect to the lateral flow assay strip between the lateral flow assay strip and the second reservoir.

36. The method of claim 34, wherein opening the seal includes puncturing the seal.

37. The method of claim 36, wherein the second reservoir is a blister, and wherein the seal is a wall of the blister.

38. The method of claim 36, wherein the seal is a frangible seal disposed in a fluidic channel between the second reservoir and the lateral flow assay strip.

39. The method of claim 38, wherein the seal is a breakable metal foil.

40. The method of claim 38, wherein the seal is a breakable film.

41. The method of claim 34, wherein fluidly connecting the first reservoir with the receptacle includes puncturing the first reservoir.

42. The method of claim 41, wherein the first reservoir is punctured with a needle.

43. The method of claim 41, wherein the first reservoir is punctured with a blade.

44. The method of claim 34, wherein the first solution is a diluent.

45. A method of performing a diagnostic test, comprising:
depositing a sample in a first reservoir of a vial;
depositing a first solution in a second reservoir of the vial, wherein the first reservoir and the second reservoir are separated by a seal that is openable to fluidly connect the first reservoir and the second reservoir;
placing the vial in a receptacle of a detection component;
opening the seal with an actuator to allow the sample and the first solution to flow to a third reservoir of the vial;
mixing the first solution and the sample in the third reservoir; and
allowing the mixed first solution and the sample to flow to a lateral flow assay strip.

46. The method of claim 45, wherein opening the seal includes puncturing the seal.

47. The method of claim 46, wherein the second reservoir is a blister, and wherein the seal is a wall of the blister.

48. The method of claim 45, wherein the seal is a frangible seal.

49. The method of claim 48, wherein the seal is a breakable metal foil.

50. The method of claim 48, wherein the seal is a breakable film.

51. The method of claim 45, wherein the first solution is a diluent.

52. A method of performing a diagnostic test, comprising:
depositing a sample in a first reservoir of a vial;
depositing a first solution in a second reservoir of the vial, wherein the first reservoir and the second reservoir are separated by a first seal that is openable to fluidly connect the first reservoir and the second reservoir;
placing the vial in a receptacle of a detection component;
opening the first seal with an actuator to allow the sample to flow to the second reservoir to mix with the first solution; and
opening a second seal positioned between the second reservoir and a third reservoir of the vial, wherein the third reservoir is in fluid communication with a lateral flow assay strip.

53. The method of claim 52, wherein opening the first seal includes puncturing the first seal.

54. The method of claim 53, wherein opening the second seal includes puncturing the second seal with the actuator.

55. The method of claim 52, wherein the first seal and the second seal are frangible seals.

56. The method of claim 55, wherein the first seal and the second seal are formed of breakable metal foil.

57. The method of claim 55, wherein the first seal and the second seal are breakable films.

58. The method of claim 52, wherein the first solution is a diluent.

59. The method of claim 52, wherein opening the second seal includes puncturing the second seal with a second actuator.

60. The method of claim 59, wherein the second actuator is configured to apply pressure to the first seal.

61. The method of claim 52, wherein the actuator is configured to apply pressure to the first seal.

62. A method of making a diagnostic test, comprising:
filling a first reservoir with a first solution, wherein the first reservoir is disposed in a housing;
placing a lateral flow assay strip in the housing;
placing a seal positioned between the first reservoir and the lateral flow assay strip, wherein the seal is configured to allow the first solution to flow from the first reservoir to the lateral flow assay strip when opened; and
providing a vial for taking a sample from a patient, wherein the vial is configured to fluidly connect to the housing, and wherein fluidly connecting the vial to the housing opens the seal.

63. The method of claim 62, further comprising placing an actuator in the housing, wherein the actuator is configured to open the seal when the vial is fluidly connected to the housing.

64. The method of claim 63, wherein the actuator is a lever configured to apply force to the first reservoir when the vial is fluidly connected to the housing.

65. The method of claim 62, wherein the housing includes a receptacle configured to receive the vial.

66. The method of claim 62, wherein the seal is a frangible seal.

67. The method of claim 66, wherein the seal is formed of breakable metal foil.

68. The method of claim 66, wherein the seal is a breakable film.

69. The method of claim 62, wherein the seal is a valve.

70. The method of claim 62, wherein the first solution is a diluent.

71. The method of claim 62, wherein the seal is a wall of the first reservoir that is ruptured under pressure when the vial is fluidly connected to the housing.


SEQUENCE LISTING (24 sequences)

SEQ ID NO: 1 - DNA, 18 bp, Artificial Sequence
  Synthetic
cggtggacaaattgtcac

SEQ ID NO: 2 - DNA, 22 bp, Artificial Sequence
  Synthetic
cttctctggatttaacacactt

SEQ ID NO: 3 - DNA, 28 bp, Artificial Sequence
  Synthetic
ttacaagcttaaagaatgtctgaacact

SEQ ID NO: 4 - DNA, 27 bp, Artificial Sequence
  Synthetic
ttgaatttaggtgaaacatttgtcacg

SEQ ID NO: 5 - DNA, 47 bp, Artificial Sequence
  Synthetic
tcagcacacaaagccaaaaatttatctgtgcaaaggaaattaaggag

SEQ ID NO: 6 - DNA, 45 bp, Artificial Sequence
  Synthetic
tattggtggagctaaacttaaagccctgtacaatccctttgagtg

SEQ ID NO: 7 - DNA, 51 bp, Artificial Sequence
  Synthetic
tcagcacacaaagccaaaaatttatttttctgtgcaaaggaaattaaggag

SEQ ID NO: 8 - DNA, 49 bp, Artificial Sequence
  Synthetic
tattggtggagctaaacttaaagccttttctgtacaatccctttgagtg

SEQ ID NO: 9 - DNA, 18 bp, Artificial Sequence
  Synthetic
tgcttcagtcagctgatg

SEQ ID NO: 10 - DNA, 22 bp, Artificial Sequence
  Synthetic
ttaaattgtcatcttcgtcctt

SEQ ID NO: 11 - DNA, 41 bp, Artificial Sequence
  Synthetic
tcagtactagtgcctgtgcccacaatcgtttttaaacgggt

SEQ ID NO: 12 - DNA, 43 bp, Artificial Sequence
  Synthetic
tcgtatacagggcttttgacatctatcttggaagcgacaacaa

SEQ ID NO: 13 - DNA, 17 bp, Artificial Sequence
  Synthetic
ctgcacttacaccgcaa

SEQ ID NO: 14 - DNA, 22 bp, Artificial Sequence
  Synthetic
gtagctggttttgctaaattcc

SEQ ID NO: 15 - DNA, 17 bp, Artificial Sequence
  Synthetic
ttgatgagctggagcca

SEQ ID NO: 16 - DNA, 18 bp, Artificial Sequence
  Synthetic
caccctcaatgcagagtc

SEQ ID NO: 17 - DNA, 41 bp, Artificial Sequence
  Synthetic
gtgtgaccctgaagactcggttttagccactgactcggatc

SEQ ID NO: 18 - DNA, 45 bp, Artificial Sequence
  Synthetic
cctccgtgatatggctcttcgtttttttcttacatggctctggtc

SEQ ID NO: 19 - DNA, 20 bp, Artificial Sequence
  Synthetic
atgtggatggctgagttgtt

SEQ ID NO: 20 - DNA, 20 bp, Artificial Sequence
  Synthetic
catgctgagtactggacctc

SEQ ID NO: 21 - DNA, 13 bp, Artificial Sequence
  Synthetic
cagccatccacat

SEQ ID NO: 22 - DNA, 30 bp, Artificial Sequence
  Synthetic
gtactgccactaaagcatacaatgtaacac

SEQ ID NO: 23 - DNA, 31 bp, Artificial Sequence
  Synthetic
aatatgcttattcagcaaaatgacttgatct

SEQ ID NO: 24 - DNA, 48 bp, Artificial Sequence
  Synthetic
  misc_feature (33)..(34): may be linked by dSpacer
cagacaaggaactgattacaaacattggccgcaattgcacaatttgcc